(19) United States
(12) Patent Application Publication
(10) Pub. No.: US 2025/0051425 A1
(43) Pub. Date: Feb. 13, 2025

(54) ANTIBODIES HAVING PAN-ANTIVIRAL ACTIVITY AND METHODS THEREOF

(71) Applicant: YALE UNIVERSITY

(72) Inventors: Akiko Iwasaki, New Haven, CT (US); Yexin Yang, Hamden, CT (US)

(21) Appl. No.: 18/719,735

(22) PCT Filed: Dec. 14, 2022
(86) PCT No.: PCT/US22/81574

§ 371 Date: Jun. 13, 2024

Related U.S. Application Data
(60) Provisional application No. 63/329,030, filed on Apr. 8, 2022.
(60) Provisional application No. 63/289,283, filed on Dec. 14, 2021.

(51) Int. Cl. C07K 16/10; A61K 39/00

(52) U.S. Cl. CPC C07K 16/1036; A61K 2039/505; C07K 2317/24; C07K 2317/33; C07K 2317/734

(57) ABSTRACT

Described herein are antibodies having pan-antiviral activity. In certain embodiments, the antibodies recognize glycosylation patterns specific to viral proteins, but not found on host proteins. Also described herein are methods of preventing, treating, and/or ameliorating viral infections using the antibodies of the disclosure.

### CROSS-REFERENCE TO RELATED APPLICATIONS

The present application claims priority under 35 U.S.C. § 119(e) to U.S. Provisional Patent Application No. 63/289,283, filed Dec. 14, 2021, and U.S. Provisional Patent Application No. 63/329,030, filed Apr. 8, 2022, which are incorporated herein by reference in their entireties.

### SEQUENCE LISTING

The ASCII text file named “047162-7343W01(01860)_Seq Listing.xml” created on Dec. 13, 2022, comprising 24.8 Kbytes, is hereby incorporated by reference in its entirety.

### BACKGROUND

Antibody therapies have been used as a treatment for viral infections since the early 20^{th} century, when sera from infected subjects who had recovered from the same infection were used. Since then, antibodies purified from the sera, monoclonal antibodies generated using the hybridoma method, and engineered antibodies (such as humanized antibodies) against viruses have also been used (Salazar et al., npj Vaccines 2, 19 (2017)).

Conventional antibodies recognize specific protein sequences or structural features found in viral surface proteins. Sequences and structures of the viral proteins, such as viral surface proteins vary considerably across viral families, species, or strains of the same species. As such, most antiviral antibodies have limited adaptability as they are specific for limited species, sometimes even strains, of viruses. Accordingly, health care facilities need to store large numbers of different antibodies, some of which have stringent storage requirements, putting further pressure on the facilities.

Therefore, there is a need for broad-spectrum antibodies that recognize viruses across multiple viral families. The present invention addresses this need.

### SUMMARY

In some aspects, the present invention is directed to the following non-limiting embodiments:

### Isolated Antigen-Binding Protein

In some aspects, the present invention is directed to an isolated antigen-binding protein.

In some embodiments, the antigen-binding protein includes a light chain polypeptide and a heavy chain polypeptide.

In some embodiments, the light chain polypeptide includes: a complementarity determining region (CDR) 1 comprising the amino acid sequence of SEQ ID NO: 1; a CDR2 comprising the amino acid sequence of SEQ ID NO: 2; and a CDR3 comprising the amino acid sequence of SEQ ID NO: 3.

In some embodiments, the heavy chain polypeptide includes: a CDR 1 comprising the amino acid sequence of SEQ ID NO: 4; a CDR2 comprising the amino acid sequence of SEQ ID NO: 5; and a CDR3 comprising the amino acid sequence of SEQ ID NO: 6.

In some embodiments, the light chain polypeptide includes the amino acid sequence of SEQ ID NO: 7, and the heavy chain polypeptide comprises the amino acid sequence of SEQ ID NO: 8.

In some embodiments, the antigen-binding protein includes at least one selected from the group consisting of a polyclonal antibody, a monoclonal antibody, a variable fragment (Fv), an antigen-binding fragment (Fab or F(ab)₂), a single chain antibody (scFv), a camelid antibody and a humanized antibody.

In some embodiments, the antigen-binding protein binds to viral proteins of two or more viruses from different viral families.

In some embodiments, the antigen-binding protein binds to a viral protein from a Orthomyxoviridae family virus and a viral protein from a Coronaviridae family virus.

In some embodiments, the antigen-binding protein binds to a viral protein from an influenza virus and a viral protein from severe acute respiratory syndrome coronavirus 2 (SARS-CoV-2).

In some embodiments, the antigen-binding protein binds to a glycosylation feature specific to a viral protein which is not found in a protein of a host of the virus.

In some embodiments, the glycosylation feature comprises an N-acetylglucosamine (GlcNAc) residue on a non-reducing end of an oligosaccharide.

### Composition

In some aspects, the present invention is directed to a composition.

In some embodiments, the composition includes an antigen-binding protein; and at least one pharmaceutically acceptable carrier.

In some embodiments, the antigen-binding protein is the same as or similar to those described above in the “Isolated Antigen-Binding Protein” section.

In some embodiments, the composition further includes: a compound for treating, preventing and/or ameliorating an infection by an influenza virus in a subject; or a compound for treating, preventing and/or ameliorating an infection by SARS-CoV-2 virus in a subject.

### Nucleic Acid Encoding Antigen-Binding Protein

In some aspects, the present invention is directed to a nucleic acid encoding an antigen-binding protein.

In some embodiments, the antigen-binding protein is the same as or similar to those described above in the “Isolated Antigen-Binding Protein” section.

In some embodiments, the nucleic acid includes a first segment including the nucleotide sequence of SEQ ID NO: 9, and a second segment including the nucleotide sequence of SEQ ID NO: 10.

Method of Preventing, Treating, and/or Ameliorating Infection

In some aspects, the present invention is directed to a method of preventing, treating and/or ameliorating an infection by a virus in a subject in need thereof.

In some embodiments, the method comprising administering to the subject an effective amount of an antibody.

In some embodiments, the antibody has pan anti-viral activity.

In some embodiments, the antibody includes an antigen-binding protein the same as or similar to those described above in the “Isolated Antigen-Binding Protein” section.

In some embodiments, a protein of the virus has a glycosylation feature is not found in a protein of a host of the virus, and the antibody specifically recognizes the glycosylation feature.

In some embodiments the glycosylation feature includes an N-acetylglucosamine (GlcNAc) residue on a non-reducing end of an oligosaccharide.

In some embodiments, the virus is an Orthomyxoviridae family virus or a Coronaviridae family virus.

In some embodiments, the virus is an influenza virus or SARS-CoV-2 virus.

In some embodiments, the subject is further administered a second compound suitable for treating, preventing and/or ameliorating an influenza virus infection, or a second compound suitable for treating, preventing and/or ameliorating COVID-19.

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the present specification is not limited to the precise arrangements and instrumentalities of the embodiments shown in the drawings.

FIG. 1 demonstrates that the recognition of Emv2 envelope protein by IGHV6-3 mAb depends on terminal GlcNAc moity, in accordance with some embodiments. Fully glycosylated gp70 (lane 1), β-N-Acetylglucosaminidase S trearted deglycosylated gp70 (lane 2), PNGase F treated deglycosylated gp70 (lane 3) and Neuraminidase/O-glycosidase treated deglycosylated gp70 (lane 4) were separated by gel electrophoresis. Proteins were transferred to PVDF membrane and blotted by IGHV6-3 mAb followed by HRP conjugated α-mouse Ig secondary antibody. Serum from Rag1^{-/-} MD4 Tg mice were used as negative control.

FIG. 2 demonstrates that IGHV6-3 mAb recognizes endogenous retroviruses (ERV) but not virus-like particles (VLP) that lack the expression of ERV envelope protein and glycogag protein, in accordance with some embodiments.

FIG. 3 demonstrates that IGHV6-3 mAb (mouse IgG2c) recognizes different recombinant viral proteins, in accordance with some embodiments. Indicated recombinant proteins were coated as antigens for ELISA, followed by detection of IGHV6-3 mAb and HRP conjugated α-mouse Ig secondary antibody. Absorbances at 450 nm were normalized to empty well incubated with primary and secondary antibodies. Serum from Rag1^{-/-} MD4 Tg mice were used as negative control.

FIG. 4 demonstrates that IGHV6-3 mAb recognizes viral particles of ERV, influenza and SARS-CoV-2, in accordance with some embodiments. Serum from Rag1^{-/-} MD4 Tg mice were used as negative control.

FIGS. 5A-5C demonstrate that natural IgM in pre-immune mice suppress ERV reactivation through complement engagement, in accordance with some embodiments. FIG. 5A: Representative flow cytometry plots of DFJ8 cells cocultured with indicated splenocytes from WT C57BL/6, Rag1^{-/-}, and Rag1^{-/-} MD4 Tg mice. ERV Env expression by DFJ8 cells was detected by mAb 573. The percentage of Env-positive cells is listed in the upper right of each plot. FIG. 5B: RT-qPCR of RNA isolated from PBMC of WT or indicated homozygote knockout mice measuring spliced ecotropic Env transcription. Values are normalized to internal GAPDH expression. AID^{-/-}, sIgM^{-/-} and C3^{-/-} mice were bred as homozygotes for over 10 generations. AID^{-/-} sIgM^{-/-} and AID^{-/-} C3^{-/-} mice were bred as homozygotes for 2-4 generations. Each data point represents an individual mouse. FIG. 5C: ELISA of serum IgM and IgG reactive to ERV particles (left panel), recombinant Env (middle panel) and recombinant Glycogag (right panel). Data are plotted as mean±SD, with individual data points representing individual mice; P-values were calculated using two-way ANOVA test with Sidik's multiple comparisons test.

FIGS. 6A-6D show that fluorescence-tagged ERV-baiting strategy enables detection of ERV-reactive B cells in pre-immune splenic and peritoneal compartments, in accordance with some embodiments. FIG. 6A: Schematic of fluorescence streptavidin-biotin-tagged ERV and VLP baiting strategy. ERV purified from ERV-infected DFJ8 cells were tagged with PE to label ERV-reactive B cells (top). DFJ8 cells infected with VSV-G pseudotyped FMLV-ΔEnvΔGlycogag were cultured for VLP production. Purified VLP were tagged with PE conjugated to AF647 dye to detect non-specific binding (bottom). FIG. 6B: Representative flow cytometry plots of DFJ8 cells transduced with FMLV-ΔEnv, FMLV-ΔGlycogag and FMLV-ΔEnvΔGlycogag for VLP production. The percentage of Env-positive cells detected by mAb 573 (top row) and of Glycogag-positive cells detected by mAb 34 (bottom row) is listed in the upper right of each plot. FIG. 6C: A representative flow cytometry plot of peritoneal B cells incubated with tagged VLP and tagged ERV (upper panel). Gated populations include: AF647⁻PE⁻ or VLP⁻ERV⁻ (lower left gate); AF647⁺PE⁺ or VLP⁺ERV⁺(upper gate); AF647⁻PE^{Hi} or VLP⁻ERV⁺(lower right gate). The percentage of gated cells is listed within each gate. ELISA of anti-Env IgM in the supernatant of FACS-sorted VLP⁻ERV⁻ and VLP⁻ERV⁺ peritoneal B cells at 5 days post cell culture (lower panel). P-values were calculated using two-tailed paired t-test. FIG. 6D: Representative flow cytometry plots of splenic (top row) and peritoneal (bottom row) B cells from naïve WT C57BL/6 mice (6-week-old) incubated with tagged VLP and tagged ERV. The gating schema for B cell subsets is shown in the first plot of each row. The percentage of VLP⁻ERV⁻, VLP⁺ERV⁺ and VLP-ERV⁺B cells is listed in each respective gate for follicular B cells and marginal zone B cells in the splenic compartment (top row), and for B-1a cells, B-1b cells and B-2 cells in the peritoneal compartment (bottom row).

FIGS. 7A-7D demonstrate that Germline-restricted IgM constituted ERV-reactive peritoneal B-1 BCR repertoire with altered Igh V_{H} gene abundance, in accordance with some embodiments. FIG. 7A: IGHV gene usage profiles are shown as the percentage of IgH sequences encoded by listed individual V_{H} genes for total and ERV-reactive peritoneal B-1 repertoires. Sequences are pooled from two mice from each group. ** (p<0.01) and **** (p<0.0001) by unpaired t-test with Holm-Sidik's multiple comparisons test. FIG. 7B: Isotype profiles are shown as the percentage of sequences encoding the listed class-switched isotypes in total and ERV-reactive peritoneal B-1 repertoires. Data are pooled from two mice from each group. FIG. 7C: The observed mutation frequencies are categorized into silent mutations (left panel) and replacement mutations (right panel) across CDR sequences in variable regions for total and ERV-reactive peritoneal B-1 repertoires. Sequences are pooled from two mice from each group. FIG. 7D: IgH gene tree-map plots showing V gene and J gene recombination in each BCR for total and ERV-reactive peritoneal B-1 repertoires. Each rectangle in the given tree-map represents a unique V(D)J recombination, and the size of each individual rectangle denotes the relative frequency of the indicated recombination. IGHV (black) and IGHJ (white) gene names are listed in each rectangle.

FIGS. 8A-8E demonstrate that ScRNA-seq identifies cluster of cells with unique gene expression pattern and large clonotype occupancy enriched in ERV-reactive B-1 cells, in accordance with some embodiments. FIG. 8A: UMAP representation of merged datasets of B-1 cells from total repertoire and B-1 cells from ERV-reactive repertoire. Assigned cluster identities are listed (left panel). Cells attributed to total (“total”) and ERV-reactive (“ERV”) repertoire are overlaid (right panel). FIG. 8B: UMAP representation of merged datasets of total and ERV-reactive B-1 cells grouped by four levels of total frequencies of unique clones: single (0<X≤1); small (1<X≤5); medium (5<X≤20); large (20<X≤100). Cells that failed to return paired heavy chain and light chain were removed from the dataset when calling clonotypes. FIG. 8C: UMAP representation of merged datasets of total and ERV⁻ reactive B-1 cells highlighting cells expressing the listed IGHV genes. FIG. 8D: Heatmap displaying the expression of top 10 differentially expressed genes (DEG) in each cluster in merged datasets of total and ERV-reactive B-1 cells. Top 10 DEG (only positive markers) were identified in each cluster compared to all remaining cells. Cluster identities are listed at the top. FIG. 8E: GO biological process analysis listing pathways that are upregulated in cluster 3, compared to the other clusters pooled.

FIGS. 9A-9E demonstrate that monoclonal antibody encoded by IGHV6-3 enhances complement-dependent neutralization of ERV through recognition of terminal GlcNAc, in accordance with some embodiments. FIG. 9A: ELISA of purified recombinant monoclonal antibodies measuring reactivities to ERV Env (left panel) and Glycogag (right panel). Serum from Rag1^{-/-} MD4 Tg mice were used as the negative control. Data are plotted as mean±SD, with individual data points representing technical replicates. The dotted line and grey filled area represent mean±2SD of the negative control. P-values were calculated using one-way ANOVA with Sidik's multiple comparisons test to the mean value of the negative control.

FIG. 9B: ELISA of IGHV6-3 mAb measuring reactivities to ERV and VLP. Data are plotted as mean±SD with individual data points representing technical replicates. P-values were calculated using two-way ANOVA with Sidik's multiple comparisons. FIG. 9C: Competitive ELISA of IGHV6-3 mAb measuring reactivities to ERV Env after incubation of IGHV6-3 mAb with indicated concentration of monosaccharides. mAb pre-absorbed with α-methylmannoside were incubated for negative control. Individual data points represent technical replicates. FIG. 9D: Western blot analysis of denatured Env (Lane 1, control), b-N-Acetylglucosaminidase S-treated Env (Lane 2), PNGase F-treated Env (Lane 3) and O-Glycosidase & Neuraminidase-treated Env (Lane 4). Bands were detected using anti-His Ab, IGHV6-3 mAb or serum from Ragl^{-/-} MD4 Tg mice (negative control). FIG. 9E: Percentage of ERV-infected DFJ8 cells quantified by mAb 573 staining in flow cytometry analysis at 48h post-infection with inhibition mediated by the addition of no mAb (negative control), mAb 83A25 (positive control), IGHV6-3 mAb or HGAC78 mAb without serum (left panel) and with serum (right panel). Data are plotted as mean±SD with individual data points representing replicate wells. P-values were calculated using one-way ANOVA with Sidik's multiple comparisons test to the mean value of the negative control.

FIGS. 10A-10D demonstrate that induction of ERV-reactive antibodies is not antigen-driven but depends on innate sensor stimulation. FIG. 10A: ELISA of Env-reactive IgM detected in the serum of WT mice receiving i.p. injection of PBS (negative control), R848, LPS or CpG. Timepoints of serum collections from the day of injection are indicated on the x-axis. Lines connecting data points over time represent individual mice. FIG. 10B: ELISA of Env-reactive IgM detected in the serum of WT mice infected with ERV i.p. (left); WT mice receiving i.p. injection of isolated T cells from TLR7^{-/-} mice (middle); WT mice infected with PR8 i.n. (right). PBS and T cells isolated from WT mice served as negative controls. Each data point represents an individual mouse. P-values were calculated using two-way ANOVA with Sidik's multiple comparisons. FIG. 10C: ELISA of Env-reactive IgM and IgG detected in the serum of WT mice housed in SPF conditions versus a germ-free facility. Each data point represents an individual mouse. P-values were calculated using two-way ANOVA with Sidik's multiple comparisons. FIG. 10D: ELISA of Env-reactive IgM and IgG detected in the serum of young (6-week-old) and old (16-week-old) mice. Each data point represents an individual mouse. P-values were calculated using two-way ANOVA with Sidik's multiple comparisons.

FIGS. 11A-11E demonstrate that a conserved glycan epitope was targeted by IGHV6-3 mAb for recognition of exogenous viruses. FIGS. 11A, 11C, and 11D: ELISA of IGHV6-3 mAb measuring reactivity to host proteins (FIG. 11A), viral proteins (FIG. 11C), and intact virus particles (FIG. 11D). Serum from Rag1-MD4 Tg mice was incubated as the negative control. Data are plotted as mean±SD, with individual data points representing technical replicates. Dotted line and grey filled area represent the mean±2SD of the negative control. P-values were calculated using two-way ANOVA with Sidik's multiple comparisons. FIG. 11B: ELISA of lectin GSL II and LEL to detect GlcNAc moieties on ERV Env and host proteins. Values are normalized to the negative controls (lectin pre-absorbed with GlcNAc). Data are plotted as mean±SD, with individual data points representing technical replicates. FIG. 11E: Western blot analysis of denatured HA (Lane 1, control), b-N-Acetylglucosaminidase S-treated HA (Lane 2), PNGase F-treated HA (Lane 3) and O-Glycosidase & Neuraminidase-treated HA (Lane 4). Bands were detected using anti-His Ab or IGHV6-3 mAb.

FIGS. 12A-12B demonstrate that emergence of ERV were established in mice with deficiency in antibody secretion and in effector molecules, in accordance with some embodiments. RT-qPCR of RNA isolated from the PBMC of WT mice or from mice with indicated genotypes measuring spliced ecotropic Env transcription. Values are normalized to internal GAPDH expression. Tlr7^{-/-} mice were bred as homozygotes for over 10 generations. Generation bred as homozygotes of AID^{-/-}sIgM^{-/-} and AID^{-/-}C3^{-/-} mice were indicated on the x-axis. Each data point represents an individual mouse.

FIGS. 13A-13B show the generation of recombinant Emv2 Env surface unit retaining epitopes for anti-Env antibody recognition, in accordance with some embodiments. FIG. 13A: Predicted transmembrane α-helices in the full-length Emv2 Env sequence (upper panel) and schematics of the full-length Env containing predicted surface unit (SU), transmembrane domain™ and signal peptide (SIG), the SU(M) containing SIG and SU, and the SU(V) containing SU. FIG. 13B: Western blot of purified recombinant SU(V) and SU(M) proteins detected using HRP-conjugated anti-His Ab or using mAb 83A25 & RP-conjugated anti-Rat IgG secondary Ab. Recombinant IL-1R proteins produced in the same expression system are the positive control for anti-His detection and the negative control for mAb 83A25 detection.

FIGS. 14A-14B demonstrate that fluorescence-tagged ERV-baiting strategy enables detection of ERV-reactive B cells in pre-immune splenic and peritoneal compartments, in accordance with some embodiments. FIG. 14A: Representative flow cytometry plots of peritoneal B cells without virions incubation, or peritoneal B cells incubated with tagged VLP, or tagged ERV, as indicated at the top of the plot. Gated populations include: AF647-PE⁻ or VLP⁻ERV⁻ (lower left); AF647⁺PE⁺ or VLP⁺ERV⁺(upper); AF647⁻PE^{Hi} or VLP⁻ERV⁺(lower right). The percentage of gated cells is listed in each gate. FIG. 14B: The percentage of VLP⁻ERV⁻ cells (left), VLP⁺ERV⁺cells (middle) and VLP⁻ERV⁺cells (right) plotted for comparison between peritoneal B-1a, peritoneal B-1b, peritoneal B-2 cells, splenic FoB and splenic MZB cells. Each data point represents an individual mouse.

FIGS. 15A-15D show that CDR3 length distribution but no other CDR3 property was different in ERV-reactive B-1 repertoire compared to total B-1 repertoire, in accordance with some embodiments. FIG. 15A: Gating schema for the FACS-sorted total naïve peritoneal repertoire (top gate) and the ERV-reactive repertoire (bottom gate). The percentage of gated cells is listed near or in gate for each plot. each plot represents the cells from an individual mouse. FIG. 15B Scaled density plot depicting CDR3 amino acid sequence length distribution profiles for the two samples of the total naïve peritoneal B-1 repertoire (Total_1 and Total 2) and of the ERV-reactive peritoneal B-1 repertoire (ERV_1 and ERV_2). Each sample was from an individual mouse. FIG. 15C: non-templated nucleotide insertion distribution patterns for BCR sequences encoded by each indicated V_{H} gene. Sequences were pooled from all four samples (Total_1, Total 2, ERV_1, ERV_2). Each sample was from an individual mouse. FIG. 15D: Calculated CDR3 length, CDR3 hydrophobicity, percentage of basic residues across CDR3, percentage of acidic residues across CDR3, of the total and the ERV⁻ reactive peritoneal B-1 repertoires. Sequences are pooled from two individual mice from each group.

FIGS. 16A-16B demonstrate that cluster 3 with expanded clones were enriched in ERV-reactive peritoneal B-1 repertoire and expressed genes related to cell survival, in accordance with some embodiments. FIG. 16A: Chord diagram depicting the clonal relationship of all clusters in ERV-reactive peritoneal B-1 repertoire (left) and total peritoneal B-1 repertoire (right). The cord represents the number of clonotypes unique and shared across the multiple clusters. Colors of cord were assigned by clusters. Sequences are pooled from two individual mice from each group. FIG. 16B: Violin plot of merged datasets of the total and the ERV-reactive B-1 cells depicting transcription level of listed genes.

FIGS. 17A-17D demonstrate that monoclonal antibodies encoded by IGHV6-3 recognize Env through terminal GlcNAc, in accordance with some embodiments. FIG. 17A: ELISA of purified recombinant monoclonal antibodies measuring the reactivities to ERV Env (left panel) and Glycogag (right panel). Serum from Rag1^{-/-} MD4 Tg mice were incubated as the negative control. All values were normalized by ratio to the signal of total antibody input (total antibody was captured by coating the plate with unlabeled anti-mouse Ig Ab, for each antibody, and were detected by the same secondary antibody). Data are plotted as mean±SD, with individual data points represents technical replicates. Dotted line and grey filled area represent mean±2SD of the negative control. P-values were calculated using one-way ANOVA with Sidik's multiple comparisons test to the mean value of negative control. FIG. 17B: Amino acid sequence alignment of the listed monoclonal antibodies across variable regions. Boxes over amino acid residues represent different amino acids compared to IGHV6-3 mAb as the reference sequence. FWRs and CDRs of IGHV6-3 mAb are indicated at the top of the alignment. Amino acid sequence in CDR3 of each mAb is indicated. FIG. 17C: Schematic of the domain-swapped antibody sequence is depicted (left). The colors were assigned to original IGHV genes. ELISA of purified recombinant domain-swapped antibodies measuring the reactivities to ERV Env (right). All values were normalized by ratio to the signal of total antibody input (total antibody was captured by coating the plate with unlabeled anti-mouse Ig Ab, for each antibody, and were detected by the same secondary antibody). Data are plotted as mean±SD, with individual data points represent technical replicates. FIG. 17D: Western blot analysis of denatured Env (Lane 1, control), b-N-Acetylglucosamine S-treated Env (Lane 2) and PNGase F-treated Env (Lane 3). Bands were detected by using anti-His Ab, HGAC78 (μ), HGAC39 (γ3) mAb or serum from Rag1MD4 Tg mice (negative control). The sequences shown in FIG. 17B are also listed below:

FWRs and CDRs of IGHV6-3:
(SEQ ID NO: 24)
EVKLEESGGGLVQPGGSMKLSCVASGFTFSNYWMNWVRQSPEKGLEWVA
QIRLKSDNYATHYAESVKGRFTISRDDSKSSVYLQMNNLRAEDTGIYYC
TDITGPIDYWGQGTTLTVSSEV
FWRs and CDRs of IGHV7-3:
(SEQ ID NO: 25)
EVKLVESGGGLVQPGGSLSLSCAASGFTFTDYYMSWVRQPPGKALEWLG
FIRNKANGYTTEYSASVKGRFTISRDNSQSILYLQMNALRAEDSATYYC
ARYYYGSSYAMDYWGQGTSVTV
FWRs and CDRs of HGAC39:
(SEQ ID NO: 26)
EVKLEESGGGLVQPGGSMKLSCVASGFTFSNYWMDWVRQSPEKGLEWVA
EIRLKSDNFATHYAESVKGRFTISRDDSKSSVYLQMNNLRAEDTGIYYC
VDLSWFAYWGQGTEVKLEESGG
FWRs and CDRs of IGHV1-53:
(SEQ ID NO: 27)
QVQLQQPGTELVKPGASVKLSCKASGYTFTSYWIHWVKQRPGQGLEWIG
NINPPNGGTNYNEKFKNKATLTVDQSSSTAYMQLSSLTSEDSAVYYCAR
KEYLWYFDVWGTGTTVTVSSQV

FIGS. 18A-18C demonstrate that secretion of ERV-reactive IgM by B-1 cells was induced by TLR activation, in accordance with some embodiments. FIG. 18A: ELISA of anti-Env IgM in the supernatant of purified peritoneal B cells of WT C57BL/6 mice and Rag1-MD4 Tg mice (negative control) after five days of cell culture. Synthetic ligands for innate immune sensors were added in the culture as indicated. Data are plotted as mean±SD, with each data point represents cells pooled from three mice. P-values were calculated using two-way ANOVA with Sidak's multiple comparisons. FIG. 18B: ELISA of anti-Env IgM and IgG in the supernatant of purified peritoneal B cells and splenic B cells of WT C57BL/6 mice after five days of cell culture. LPS and R848 were added in the culture as indicated. Data are plotted as mean±SD, with each data point represents cells pooled from two mice. P-values were calculated using two-way ANOVA with Sidik's multiple comparisons. FIG. 18C: RT-qPCR of RNA isolated from the PBMC of TLR7-competent F1 mice (heterozygous littermates), TLR7-deficient F1 mice without ERV viremia, TLR7-deficient F1 mice with ERV viremia, and TLR7-competent mice with ERV viremia (AID^{-/-}C3^{-/-}F3), measuring spliced ecotropic Env transcription (left). Values are normalized to internal GAPDH expression. ELISA of IgM reactive to Env in the serum collected from the same group of mice. Each data point represents an individual mouse.

FIG. 19 is a matric summary for 5′ gene expression single cell profiling in accordance with some embodiments.

FIG. 20 is a matric summary for V(D)J repertoire single cell profiling in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The study described herein (“the present study”) produced exemplary antibodies according to the present invention that have pan antiviral activity. In certain embodiments, these exemplary antibodies are able to recognize viral proteins across several virus families. In certain embodiments, the antibodies recognize glycosylation patterns specific to viral proteins (e.g., the N-acetylglucosamine (GlcNAc) residues on the non-reducing end of oligosaccharides), rather than specific peptide sequences, which vary significantly among viral families. These glycosylation features are conserved glycan epitopes enriched in a broad range of viral antigens. The specificities against such conserved glycan modification ensure the recognition of various viral antigens despite the broad diversity of the peptide sequences of the viral proteins.

Indeed, the present study demonstrated that the pan antiviral antibodies herein are able to recognize surface proteins of influenza viruses, which are from the Orthomyxoviridae viral family, and surface proteins of SARS-CoV-2, which is from the Coronaviridae family. The surface proteins of influenza viruses and the surface proteins of SARS-CoV-2 share little similarities, except that they all have certain glycosylation features recognized by the present antibodies.

Furthermore, since certain antibodies of the disclosure recognize terminal GlcNAc moieties but not GlcNAc in the core structure of oligosaccharide, in certain embodiments these antibodies pose minimal risk of autoreactivity because glycan chains on host proteins are capped with sialic acid.

Accordingly, in some embodiments, the present specification is directed to an antibody.

In some embodiments, the present specification is directed to a composition including an antibody.

In some embodiments, the present specification is directed to a nucleic acid that encodes an antibody.

In some embodiments, the present specification is directed to a method of detecting a virus.

In some embodiments, the present specification is directed to a method of neutralizing a virus.

In some embodiments, the present specification is directed to a method of treating, ameliorating, and/or preventing a viral infection.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %,4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %. As used herein, the term “affinity” for a molecule towards another refers to the degree (or tightness) of binding between the two molecules. A higher affinity means tighter binding between the two molecules. Affinity can be quantified in terms of dissociation constant (or K_{d}), where a K_{d} value that is lower in magnitude (closer to zero) indicates a higher affinity.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, can be modified by methylation, amidation, acetylation or substitution with other chemical groups which can change a peptide's circulating half-life without adversely affecting activity of the peptide. Additionally, a disulfide linkage may be present or absent in the peptides.

The term “antibody” or “Ab” or “immunoglobulin” are terms of art and can be used interchangeably and refer to a protein, or polypeptide sequence which is or is derived from an immunoglobulin molecule having at least one antigen binding site which specifically binds to a specific epitope on an antigen (See, e.g., Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, hetero-conjugate antibodies, single domain antibodies, monovalent antibodies, single chain antibodies or single chain Fv (scFv), nanobodies, intracellular antibodies, intrabodies, camelized antibodies, camelid antibodies, IgNAR antibodies, affybodies, Fab fragments, F(ab′) fragments, F(ab)₂, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. Full-length antibodies are sometimes tetramers comprising two heavy chain and two light chain immunoglobulin molecules.

As used herein, the term “monoclonal antibody” refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody encoded by identical nucleic acid sequence, originating from a single clone of cells or cell line, wherein the antibody binds to a glycosylation feature specific to viral proteins as determined, e.g., by ELISA or other antigen-binding or competitive binding assay known in the art. In particular embodiments, a monoclonal antibody can be a chimeric antibody, a human antibody, or a humanized antibody. Methods for generating a humanized antibody are known in the art. In certain embodiments, a monoclonal antibody is a monovalent antibody or multivalent (e.g., bivalent) antibody. In particular embodiments, a monoclonal antibody is a monospecific or multi-specific antibody (e.g., bispecific antibody). Monoclonal antibodies described herein can, for example, can be made by the hybridoma method as described in Kohler et al.; Nature, 256:495 (1975) or can be isolated from phage libraries, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Monoclonal antibodies may be identified by high-throughput direct sequencing of fully recombined VDJ sequences of B cell receptor (BCR) repertoires from single cells of animals immunized with an antigen for which the desired monoclonal antibody will specifically bind as described herein. See, e.g., Goldstein et al., Communications Biology (2019)2:304; Horns et al., Cell Reports (2020) 30:905-913). The identified monoclonal antibodies are then produced recombinantly.

The terms “antibody fragment,” “antigen-binding fragment,” and “antigen-binding domain” of an antibody and similar terms are used interchangeably and refer to at least one portion of an intact antibody, or recombinant variants thereof, and comprising or consisting of the antigen-binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), VHH domains, and multi-specific (e.g., bispecific) antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-V_{H} or may comprise V_{H}-linker-VL.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

As used herein, the term “constant region” or “constant domain” refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

As used herein, the terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs).

By the term “recombinant antibody” as used herein, is meant an antibody which is generated using recombinant DNA technology, such as, for example, an antibody expressed by a bacteriophage, insect, or yeast expression system or by a human cell line expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence.

The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated or synthesized, or can be derived from a biological sample. Such a biological sample can include, but is not limited to a tissue sample, a tumor sample, a cell or a biological fluid.

As used herein, an “epitope” is a term in the art and refers to a localized region of an antigen to which an antibody can specifically bind. An epitope can be, for example, contiguous amino acids of a polypeptide (linear or contiguous epitope), or an epitope can, for example, come together from two or more non-contiguous regions of a polypeptide or polypeptides (conformational, non-linear, discontinuous, or non-contiguous epitope), or an epitope can be, for example, glycan and lipid groups from post-translational modification, such as on the amino acids of a polypeptide. In certain embodiments, the epitope can be determined by structural methods, e.g., X-ray diffraction crystallography, nuclear magnetic resonance (NMR), or electron microscopy (e.g., negative stain or cryo-EM), ELISA assays, hydrogen/deuterium exchange coupled with mass spectrometry (e.g., MALDI mass spectrometry), array-based oligo-peptide scanning assays, and/or mutagenesis mapping (e.g., site-directed mutagenesis mapping). In a specific embodiment, the epitope of an antibody or antigen-binding fragment is determined using cryo-EM studies, such as described herein.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (K_{D}). Affinity can be measured and/or expressed in a number of ways known in the art, including, but not limited to, equilibrium dissociation constant (K_{D}), equilibrium association constant (K_{A}), and IC50. The K_{D} is calculated from the quotient of k_{off}/kₒₙ, whereas K_{A} is calculated from the quotient of kₒₙ/k_{off}, where kₒₙ refers to the association rate constant of, e.g., an antibody to an antigen, and k_{off} refers to the dissociation rate constant of, e.g., an antibody to an antigen.

The term “avidity” as used herein refers to the total binding strength of an antibody for an antigen at every binding site in a single non-covalent interaction, which affects the functional affinity.

As used herein, a “neutralizing” antibody or antigen-binding fragment is an antibody that defends a cell from a pathogen or infectious particle such as a virus by neutralizing any effect it has biologically. Neutralization renders the particle no longer infectious or pathogenic. By binding specifically to surface structures (antigen) on an infectious particle, neutralizing antibodies prevent the particle from interacting with its host cells it might otherwise infect and destroy. Neutralizing antibodies can inhibit the infectivity of the pathogen by binding to the pathogen and blocking the molecules needed for cell entry. This can be due to the antibodies sterically interfering with the pathogens or toxins attaching to host cell receptors. In case of a virus infection, neutralizing antibodies can bind, e.g., to glycoproteins of enveloped viruses or capsid proteins of non-enveloped viruses. Furthermore, neutralizing antibodies can act by preventing particles from undergoing structural changes often needed for successful cell entry. For example, neutralizing antibodies can prevent conformational changes of viral proteins that mediate the membrane fusion needed for entry into the host cell. In some cases, the virus is unable to infect even after the antibody dissociates. The pathogen-antibody complex is eventually taken up and degraded by host macrophages.

A “coding region” of a gene consists of the nucleotide residues of the coding strand of the gene and the nucleotides of the non-coding strand of the gene that are homologous with or complementary to, respectively, the coding region of an mRNA molecule produced by transcription of the gene.

A “coding region” of an mRNA molecule also consists of the nucleotide residues of the mRNA molecule that are matched with an anti-codon region of a transfer RNA molecule during translation of the mRNA molecule or that encode a stop codon. The coding region may thus include nucleotide residues corresponding to amino acid residues not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “delivery vehicle” is used herein as a generic reference to any delivery vehicle capable of delivering a compound to a subject, including, but not limited to, dermal delivery vehicles and transdermal delivery vehicles.

The term “DNA” as used herein is defined as deoxyribonucleic acid.

“Effective amount” or “therapeutically effective amount” are used interchangeably herein, and refer to an amount of a compound, formulation, material, or composition, as described herein, effective to achieve a particular biological result. Such results may include, but are not limited to, treatment of a disease or condition as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length, at least about 200 amino acids in length, at least about 300 amino acids in length, and at least about 400 amino acids in length (and any integer value in between). As used herein, an antibody fragment refers to active fragments thereof, i.e., fragments having the same or similar characteristics that are used for the definition of an antibody according to the invention, in certain embodiments affinity for viral glycosylation features. For convenience when the term antibody is used, fragments thereof exhibiting the same characteristic are also being considered.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

An “individual”, “patient” or “subject”, as that term is used herein, includes a member of any animal species including, but are not limited to, birds, humans and other primates, and other mammals including lab animals such as mice, rats, and rabbits, and commercially relevant mammals such as cattle, pigs, horses, sheep, cats, and dogs. Sometimes, the subject is a human.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “protein”, “peptide” and “polypeptide” are used interchangeably, and refer to a compound comprised of amino acid residues covalently linked by peptide bonds. The term “peptide bond” means a covalent amide linkage formed by loss of a molecule of water between the carboxyl group of one amino acid and the amino group of a second amino acid. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Proteins” include, for example, biologically active fragments, substantially homologous proteins, oligopeptides, homodimers, heterodimers, variants of proteins, modified proteins, derivatives, analogs, and fusion proteins, among others. The proteins include natural proteins, recombinant proteins, synthetic proteins, or a combination thereof. A protein may be a receptor or a non-receptor.

The term “recombinant DNA” as used herein is defined as DNA produced by joining pieces of DNA from different sources.

The term “recombinant polypeptide” as used herein is defined as a polypeptide produced by using recombinant DNA methods.

The term “RNA” as used herein is defined as ribonucleic acid.

The term “therapeutic” as used herein means a treatment and/or prophylaxis.

The term to “treat,” as used herein, means reducing the frequency with which symptoms are experienced by a subject or administering an agent or compound to reduce the frequency and/or severity with which symptoms are experienced. As used herein, “alleviate” is used interchangeably with the term “treat.”

As used herein, “treating a disease, disorder or condition” means reducing the frequency or severity with which a symptom of the disease, disorder or condition is experienced by a subject. Treating a disease, disorder or condition may or may not include complete eradication or elimination of the symptom.

The following abbreviations are used herein: CDR, complementary-determining region; VH, heavy chain variable region; VL, light chain variable region.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Antibody

In some embodiments, the present specification is directed to an antibody. In some embodiments, the present specification is directed to a pan antiviral antibody, or an antibody that recognizes viruses from multiple (i.e., more than one) viral families. In some embodiments, the antibody that recognizes a glycosylation feature specific to a viral protein, such as a viral surface protein, which is not present on a host protein.

In some embodiments, the glycosylation feature specific to the viral protein includes a N-acetylglucosamine (GlcNAc) residue on a non-reducing end of oligosaccharides.

In some embodiments, the antibody recognizes a viral protein from an Orthomyxoviridae family virus and a viral protein from a Coronaviridae family virus. In some embodiments, the antibody recognizes a viral protein from an influenza virus and a viral protein from severe acute respiratory syndrome coronavirus 2 (SARS-CoV-2).

In some embodiments, the antibody recognizes a viral particle, such as a virus belonging to the Orthomyxoviridae family and a virus belonging to the Coronaviridae family.

In some embodiments, the antibody recognizes an influenza virus and severe acute respiratory syndrome coronavirus 2 (SARS-CoV-2).

In some embodiments, the antibody includes an immunoglobulin light chain variable region (VL) and an immunoglobulin heavy chain variable region (VH).

In some embodiments, the VL includes a complementary-determining region (CDR) 1 including the amino acid sequence of SEQ ID NO: 1 (QDINSY), a CDR2 region including the amino acid sequence of SEQ ID NO: 2 (RAN), and a CDR3 region including the amino acid sequence of SEQ ID NO: 3 (LQYDEFPYT).

In some embodiments, the VH includes a CDR1 including the amino acid sequence of SEQ ID NO: 4 (GFTFSNYW), a CDR2 region including the amino acid sequence of SEQ ID NO: 5 (IRLKSDNYAT), and a CDR3 region including the amino acid sequence of SEQ ID NO: 6 (TDITGPIDY).

In some embodiments, the VL includes the amino sequence of SEQ ID NO: 7 (MDMRTPAQFLGILLLWFPGIKCDIKMTQSPSSMYASLGERVTITCKASQDINSYLSW FQQKPGKSPKTLIYRANRLVDGVPSRFSGSGSGQDYSLTISSLEYEDMGIYYCLQYDE FPYTFGGGTKLEIK).

In some embodiments, the VH includes the amino acid sequence of SEQ ID NO: 8 (MDLRLSCAFIIVLLKGVQSEVKLEESGGGLVQPGGSMKLSCVASGFTFSNYWMNW VRQSPEKGLEWVAQIRLKSDNYATHYAESVKGRFTISRDDSKSSVYLQMNNLRAED TGIYYCTDITGPIDYWGQGTTLTVSS).

In some embodiments, the antibody includes a polyclonal antibody, a monoclonal antibody, a variable fragment (Fv), an antigen-binding fragment (Fab or F(ab)₂), a single chain antibody (scFv), a camelid antibody, a humanized antibody, or combinations thereof.

### Composition

In some embodiments, the present specification is directed to a composition, such as a pharmaceutical composition, such as a composition for preventing, treating and/or ameliorating a viral infection.

In some embodiments, the composition includes an antibody and at least one pharmaceutically acceptable carrier. In some embodiments, the antibody is an antibody that recognizes a virus. In some embodiments, the virus is a virus having a protein glycosylation, such as a surface protein glycosylation. In some embodiments, the glycosylation of the viral protein, such as the viral surface protein includes an N-acetylglucosamine (GlcNAc) residue on a non-reducing end of oligosaccharides. In some embodiments, the antibody recognizes the GlcNAc residue on the non-reducing end of the oligosaccharides. In some embodiments, the antibody is the same as or similar to those as described elsewhere herein.

In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration. The administration and dosage of the composition is also described elsewhere herein.

In some embodiments, the composition further includes a second compound for treating, preventing and/or ameliorating the viral infection, such as influenza infection and/or SARS-CoV-2 infection. Examples of the second compound is described elsewhere herein.

Nucleic Acid that Encodes Antibody

In some embodiments, the specification is directed to a nucleic acid that encodes an antibody. In some embodiments, the antibody is the same as or similar to those as described elsewhere herein.

In some embodiments, In some embodiments, the nucleic acid includes a first segment encoding a light chain portion of the antibody, and the first segment includes a sequence of SEQ ID NO: 9

(ATGGACATGAGGACCCCTGCTCAGTTTCTTGGAATCTTGTTGCTCTGG
TTTCCAGGTATCAAATGTGACATCAAGATGACCCAGTCTCCATCTTCCA
TGTATGCATCTCTAGGAGAGAGAGTCACTATCACTTGCAAGGCGAGTCA
GGACATTAATAGCTATTTAAGCTGGTTCCAGCAGAAACCAGGGAAATCT
CCTAAGACCCTGATCTATCGTGCAAACAGATTGGTAGATGGGGTCCCAT
CAAGGTTCAGTGGCAGTGGATCTGGGCAAGATTATTCTCTCACCATCAG
CAGCCTGGAGTATGAAGATATGGGAATTTATTATTGTCTACAGTATGAT
GAGTTTCCGTACACGTTCGGAGGGGGGACCAAGCTGGAAATAAAAC).

In some embodiments, the nucleic acid includes a second segment encoding a heavy chain portion of the antibody, and the second segment includes a sequence of SEQ ID NO: 10

(ATGGACTTGAGACTGAGCTGTGCTTTTATTATTGTTCTTTTAAAAGGG
GTCCAGAGTGAAGTGAAGCTTGAGGAGTCTGGAGGAGGCTTGGTGCAAC
CTGGAGGATCCATGAAACTCTCCTGTGTTGCCTCTGGATTCACTTTCAG
TAACTACTGGATGAACTGGGTCCGCCAGTCTCCAGAGAAGGGGCTTGAG
TGGGTTGCTCAAATTAGATTGAAATCTGATAATTATGCAACACATTATG
CGGAGTCTGTGAAAGGGAGGTTCACCATCTCAAGAGATGATTCCAAAAG
TAGTGTCTACCTGCAAATGAACAACTTAAGGGCTGAAGACACTGGAATT
TATTACTGCACAGATATAACTGGGCCGATTGACTACTGGGGCCAAGGCA
CCACTCTCACAGTCTCCTCAG).

In some embodiments, the nucleic acid that encodes the antibody is part of a vector, such as an expression vector, a viral vector, or a mammalian cell vector.

### Method of Detecting Virus

In some embodiments, the present specification is directed to a method of detecting a virus. In some embodiments, the method includes contacting a sample suspected to include the virus with an antibody that recognize the virus, and visualizing the antibody. In some embodiments, a presence of a signal of the antibody indicates a presence of the virus, and/or an absence of the signal of the antibody indicates an absence of the virus. In some embodiments, the antibody and/or the viruses that the antibody recognize are the same as or similar to those as described elsewhere herein.

In some embodiments, visualizing the antibody includes visualizing the antibody using an immunostaining technique. Non limiting examples of immunostaining techniques includes an immunohistochemistry staining, flow cytometry, western blotting, an enzyme-linked immunosorbent assay (ELISA), and immuno-electron microscopy.

### Method of Neutralizing Virus

In some embodiments, the present specification is directed to a method of neutralizing a virus. In some embodiments, the method includes contacting the virus with an antibody. In some embodiments, the antibody and/or the viruses that the antibody recognize are the same as or similar to those as described elsewhere herein. In some embodiments, the virus is further contacted with one or more complement proteins.

In some embodiments, the virus is in a cell, such as an isolated cell, a cultured cell, and/or a cell in a subject.

In some embodiments, the virus is in a subject. In some embodiments, the subject is infected with the virus. In some embodiments, the subject is a mammal, such as a human.

Method of Preventing, Treating, and/or Ameliorating Viral Infection

In some embodiments, the present specification is directed to a method of preventing, treating, and/or ameliorating a viral infection in a subject in need thereof. In some embodiments, the method includes administering to the subject an effective amount of an antibody. In some embodiments, the antibody and/or the viruses that the antibody recognize are the same as or similar to those as described elsewhere herein.

In some embodiments, the viral infection is a viral infection by a virus of the Orthomyxoviridae family and/or a virus of the Coronaviridae family. In some embodiments, the viral infection is a viral infection by an influenza virus and/or a viral infection by SARS-CoV-2 virus.

In some embodiments, the subject is further administered with a second compound.

In some embodiments, the second compound is compound suitable for treating, preventing and/or ameliorating an influenza virus infection, or a compound suitable for treating, preventing and/or ameliorating COVID-19.

### Methods of Targeting Terminal GlcNAc Epitopes

Terminal GlcNAc epitopes exist in bacteria and viruses. Furthermore, such glycan epitopes have been identified on malignant cells and in autoimmune disease. As such, targeting terminal GlcNAc epitopes by antigen binding proteins that recognize these epitopes can be used to detect and/or neutralize bacteria, viruses, malignant cells and/or cells involved in autoimmune diseases that express such epitopes.

Accordingly, in some aspects, the present invention is directed to a method of targeting a terminal GlcNAc epitope. In some embodiments, the method is a method of detecting a bacterium, a viruse, a malignant cell and/or a cell involved in autoimmune diseases that express terminal GlcNAc epitopes. In some embodiments, the method is a method of neutralizing a bacterium, a virus, a malignant cell and/or a cell involved in autoimmune diseases that express terminal GlcNAc epitopes. In some embodiments, the method is a method of treating a disease, disorder and/or condition that is caused by a bacterium, a virus and/or a cell that express terminal GlcNAc epitopes.

In some embodiments, the method includes contacting the bacterium, the virus, the malignant cell and/or the cell involved in the autoimmune disease an antibody recognizing a terminal GlcNAc epitope. In some embodiments, the antibody is the same as or similar to those as described elsewhere herein.

In some embodiments, the bacterium, the virus, the malignant cell and/or the cell involved in the autoimmune disease is further contacted with one or more complement proteins.

### Combination Therapies

The compounds useful within the methods described herein can be used in combination with one or more additional therapeutic agents useful for preventing, treating and/or ameliorating viral infection. These additional therapeutic agents may comprise compounds that are commercially available or synthetically accessible to those skilled in the art. These additional therapeutic agents are known to treat, prevent, and/or reduce the symptoms, of the viral infection. These additional therapeutic agents are administered before, at the same time with, or after the administration of the present antibody.

In certain embodiments, the compounds described herein can be used in combination with second compound for treating, preventing and/or ameliorating the viral infection.

For example, in the case of an infection by an influenza virus, the second compound includes a compound suitable for treating, preventing and/or ameliorating an influenza virus infection. In some embodiments, the compound includes an influenza vaccine that immunizes the subject against influenza infection; a decongestant that relieves nasal congestion, swelling, and runny nose; a cough medication that blocks the cough reflex, thin and loosen mucus; a nonsteroidal anti-inflammatory drug (NSAID) that relieves pain, decreases inflammation, and reduces fever; an analgesic that relieves pain; and/or an antiviral drug against influenza virus, such as oseltamivir, zanamivir, peramivir, baloxavir marboxil, and derivatives thereof.

For example, in the case of an infection by SARS-CoV-2, the second compound includes a compound suitable for treating, preventing and/or ameliorating COVID-19. In some embodiments, the compound comprises an COVID-19 vaccine that immunizes the subject against SARS-CoV-2; a cough medication that blocks the cough reflex, thin and loosen mucus; a nonsteroidal anti-inflammatory drug (NSAID) that relieves pain, decreases inflammation, and reduces fever; an analgesic that relieves pain; an antibody (other than the present antibody) that neutralizing SARS-CoV-2 virus; an immunomodulator such as a corticosteroid (e.g., dexamethasone), an interleukin (IL-6) inhibitor (e.g., tocilizumab or sarilumab), a Janus kinase (JAK) inhibitor (baricitinib or tofacitinib); and/or an antiviral drug against SARS-CoV-2, such as remdesivir. The compounds suitable for treating SARS-CoV-2 infections are also detailed in the “Coronavirus Disease 2019 (COVID-19) Treatment Guidelines” by the National Institutes of Health (NIH), the entirety of which is hereby incorporated herein by reference.

In various embodiments, a synergistic effect is observed when a compound as described herein is administered with one or more additional therapeutic agents or compounds. A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination.

The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated herein (in a non-limiting aspect, a viral infection). Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat, prevent and/or ameliorate a viral infection in the patient. An effective amount of the therapeutic compound necessary to achieve a therapeutic effect may vary according to factors such as the state of the disease or disorder in the patient; the age, sex, and weight of the patient; and the ability of the therapeutic compound to treat, prevent and/or ameliorate a viral infection in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, ameliorate, and/or reduce one or more symptoms of viral infection in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol.

Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In certain embodiments, the compound(s) described herein are administered to a patient, alone or in combination with another pharmaceutical agent, using a sustained release formulation. In certain embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of compounds described herein depends on the age, sex and weight of the patient, the current medical condition of the patient and the progression of a viral infection in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Another suitable dose of a compound described herein can be in the range of from about 0.01 mg/kg to about 100 mg/kg per day or about 0.1 g/kg to about 100 g/kg per day, wherein the amount of the compound administered depends on the weight of the patient. The dose of compounds described herein can be about 0.1 mg/kg to about 100 mg/kg, about 0.5 mg/kg to about 100 mg/kg, about 1 mg/kg to about 50 mg/kg, 0.5 mg/kg to about 25 mg/kg.

In some embodiments, the dose of a compound described herein can be greater than, less than, or at least about 0.01 mg/kg, 0.1 mg/kg, 0.5 mg/kg, 1 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or about 100 mg/kg. The dose of compounds described herein can be about 0.1 g/kg to about 100 g/kg, about 0.5 g/kg to about 100 g/kg, about 1 g/kg to about 50 g/kg, 0.5 g/kg to about 25 μg/kg. In some embodiments, the dose of a compound described herein can be greater than, less than, or at least about 0.1 μg/kg, 0.5 μg/kg, 1 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, or about 100 vg/kg.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### Examples

The present specification further describes in detail by reference to the following experimental examples. These examples are provided for purposes of illustration only, and are not intended to be limiting unless so specified. Thus, the present specification should in no way be construed as being limited to the following examples, but rather, should be construed to encompass any and all variations which become evident as a result of the teaching provided herein.

### Example 1: IGHV6-3 monoclonal antibody

A pool of innate-like B cells known as B-1 cells encode germline restricted B cell receptors and secrete natural antibodies, functioning as the first line of defense in addition to adaptive conventional B cells. The unmutated B cell receptors were selected in prenatal and perinatal stage to target conserved antigens present on a broad range of pathogens. Endogenous retroviruses (ERVs) are remnants in the host genome of ancient retroviral infections and germline integration. Expression of antigens derived from ERV sequences early in the development of the host immune system may provide positive cues for selection of B-1 cells harboring potential to recognize conserved epitopes on viral proteins. The present study therefore seeks to screen for natural antiviral antibodies and to evaluate the breadth of viral recognition.

An antigen-baiting system was established to label and sort endogenous retrovirus reactive B-1 cells in naïve C57BL/6 mice. The antigen-baiting system is consisted of two major component—ERV bait and virus-like particle (VLP)—control. The ERV-bait is phycoerythrin labeled ERV particles to mark B cells interacted with ERV. The VLP-control is phycoerythrin-Alexa fluor 647 dye labeled virus-like particles lacking envelope protein and glycosylated gag protein to block non-specific interaction. After sorting out ERV-reactive B-1 cells, single cell sequencing via 10× genomics platform was performed and repertoire analysis of bait-enriched B cell receptors was conducted to reveal potential viral reactive antibody sequences. Top hits of antibody heavy chain variable region sequences overrepresented in bait-enriched B cells were synthesized and subcloned into expression plasmid pRVL-2 (addgene plasmid #104580) containing antibody constant region for expression of IgG2c monoclonal antibodies in Expi293 cells expression system. Paired antibody light chain variable region sequences were synthesized and subcloned into expression plasmid pRVL-1 (addgene plasmid #104579) containing κ light chain constant region. Expi293 cells were transiently transfected with antibody expression plasmid to generate desired monoclonal antibodies. Antibodies purified by protein G affinity chromatography were tested for specificities against various viral antigens using ELISA. It was found that one of the monoclonal antibodies (variable region sequence provided) named IGHV6-3 mAb recognized various viral antigens including ERV envelope protein, ERV glycosylated gag protein, severe acute respiratory syndrome coronavirus 2 (SARS-CoV-2) spike protein and influenza hemagglutinin protein. It was further found that the broad viral reactivity of this monoclonal antibody is based on the recognition of terminal N-acetylglucosamine (GlcNAc) residues enriched on viral protein but not on host protein.

### Example 1-1: IGHV6-3 mAb recognizes N-Acetylglucosamine (GlcNAc) on the non-reducing end of N-linked glycan

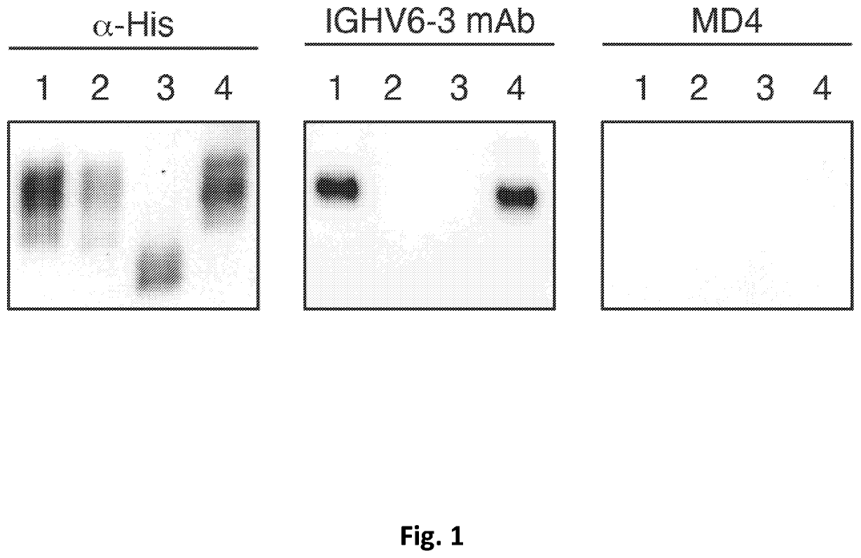

IGHV6-3 encoded heavy chain variable region was subcloned into pRVL-2 backbone (addgene plasmid #104580) containing mouse IgG2c constant region, and IGKV14 encoded light chain variable region was subcloned into pRVL-1 backbone (addgene plasmid #104579) containing mouse Igκ constant region. Expi293 cells were transfected by IGHV6-3-pRVL-2 and IGKV14-pRVL-1 and were cultured for antibody production post transfection. IGHV6-3 monoclonal antibody (mAb) was harvested and purified by protein G based affinity chromatography. To test glycan dependency of antigen recognition by IGHV6-3 mAb, purified recombinant ERV envelope protein Emv2 gp70 was pretreated by the following deglycosylation enzyme before gel electrophoresis and detection by IGHV6-3 mAb. 3-N-Acetylglucosaminidase S catalyzes the hydrolysis of terminal non-reducing p-N-Acetylglucosamine residues from oligosaccharides. PNGase F cleaves between the innermost GlcNAc and asparagine residues of high mannose, hybrid, and complex oligosaccharides on N-linked glycans. Neuraminidase and O-glycosidase remove O-linked glycosylation. As shown in FIG. 1, it was found that the recognition of gp70 by IGHV6-3 mAb was dependent on terminal GlcNAc moieties on N-linked glycans instead of epitopes presented by the peptide sequences, as the removal of terminal GlcNAc and N-linked glycosylation completely abolished antibody recognition.

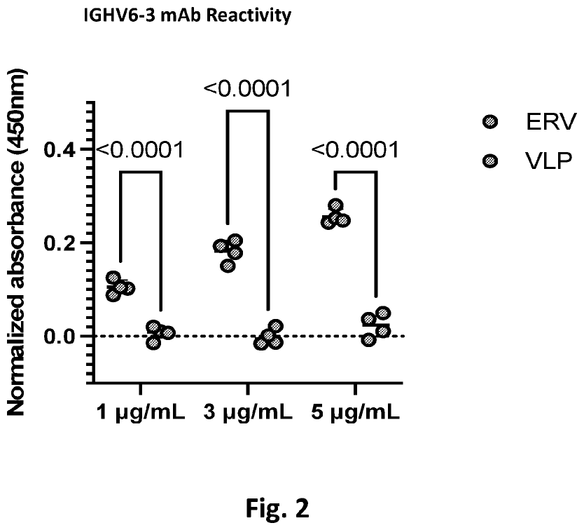

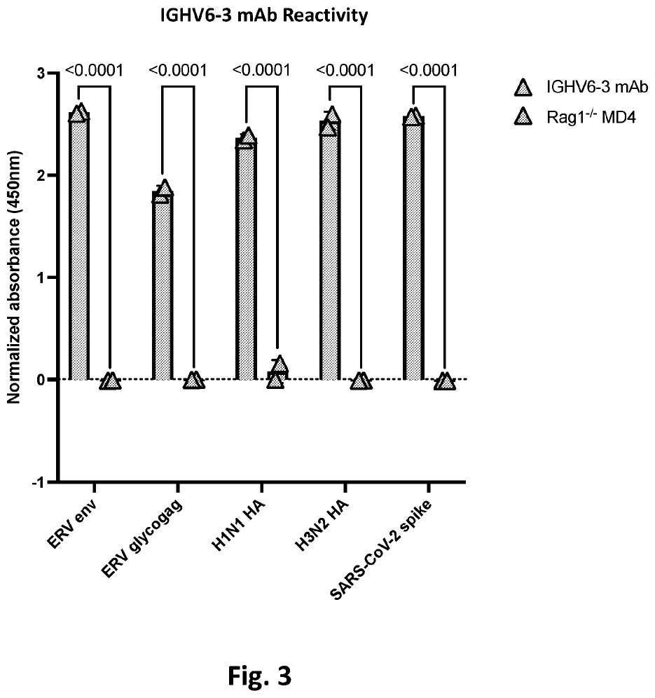

Notably, only terminal GlcNAc residues but not GlcNAc in the core structures of glycan chains was removed by β-N-Acetylglucosaminidase S treatment. The results suggested that IGHV6-3 mAb exclusively recognized terminal GlcNAc residues, which are not present on normal host proteins. Whether IGHV6-3 mAb can recognize host derived components was further tested by performing ERV and VLP coated ELISA with IGHV6-3 mAb. Antigens were coated on microplate at 4° C. overnight. IGHV6-3 mAb antibodies were incubated with coated plate at 4° C. overnight and antibodies bound to coated antigens were detected by HRP conjugated α-mouse Ig secondary antibodies. Antibodies reactivity against coated antigens were measured by 450 nm absorbance in antigen coated well. All values are normalized to 450 nm absorbance in wells coated with coating buffer for control. VLP lacks two major ERV derived surface antigens—envelope protein and glycosylated gag protein, while contains host derived membrane component. It was found that at the concentration of 1[μg/mL, 3p g/mL and 5 μg/mL, IGHV6-3 mAb only recognize ERV but not VLP (FIG. 2).

### Example 1-2: IGHV6-3 mAb recognizes viral antigens across several virus families

Considering that GlcNAc reactive IGHV6-3 mAb targeted exclusively the glycan modification on the antigens and a wide range of viral proteins are generally modified by conserved glycosylation, it was hypothesized that IGHV6-3 mAb could harbor cross-reactivity to other viral antigens beyond endogenous retrovirus. To test this, antigen coated enzyme-linked immunosorbent assay (ELISA) was performed with IGHV6-3 mAb and reactivity against coated viral proteins was measured using sane strategy as described above. The result showed that IGHV6-3 mAb recognized ERV envelope protein, ERV glycosylated gag protein, H1N1 and H3N2 hemagglutinin proteins, and SARS-CoV-2 spike protein (FIG. 2).

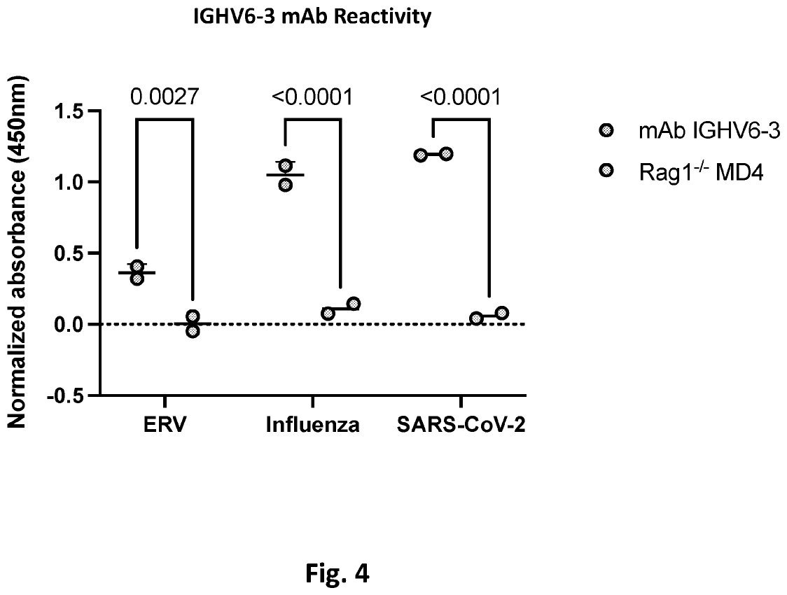

To further test whether IGHV6-3 mAb can recognize live viral particles in addition to purified viral antigens, whole viral particles coated ELISA was performed with IGHV6-3 mAb and reactivity against coated virions was measured using same strategy as described above. It was confirmed that IGHV6-3 mAb can recognize live ERV, influenza and SARS-CoV-2 virions (FIG. 4).

### Example 2: Identification and characterization of IGHV6-3 monoclonal antibody

Example 2 is directed to a comprehensive study which lead to the isolation and characterization of IGHV6-3 monoclonal antibody.

Endogenous retroviruses (ERV), comprising a substantial portion of the vertebrate genome, are remnants of ancient genetic invaders. ERV with near-intact coding potential reactivate in B cell-deficient mice. Here, the present study employed an antigen-baiting strategy to enrich B cells reactive to ERV surface antigens. The present study identified ERV-reactive B-1 cells expressing germline-encoded natural IgM antibodies in naïve mice, the level of which further increases upon innate immune sensor stimulation. B cell receptor repertoire profiling of ERV-reactive B-1 cells revealed increased usage of Igh V_{H} gene that gives rise to glycan-specific antibodies targeting terminal N-acetylglucosamine moieties on ERV glycoproteins, which further engage complement pathway to protect the host from ERV emergence. These same antibodies also recognize glycoproteins of other enveloped viruses, but not self-proteins. These results reveal an innate antiviral mechanism of germline-encoded antibodies with broad reactivity to enveloped viruses, whose absence leads to the emergence of infectious ERV.

### Example 2-1: Related information and brief summary

After endogenization of proviral sequences into the host germline, retroviruses gain the capacity to be vertically transmitted through integration into host genetic material. These events are infrequent but have cumulatively resulted in 8-10% occupancy of murine and human genomes by endogenous retroviruses (ERV). The majority of ERV loci are subject to open reading frame (ORF) degeneration, leaving few copies with intact coding capacity. The proviral ERV as the endogenized counterpart of the exogenous murine leukemia virus (MLV), are actively transcribed and translated in common laboratory mouse strains. Proviral ERV contain gag, pol and env gene flanked by two long-terminal repeats (LTR). ERV has established a dynamic equilibrium during co-evolution with the host, balancing their genetic transmission while undergoing a constant arms race with the host control machinery. Despite multiple cell-intrinsic mechanisms function to suppress ERV loci and restrict cellular synthesis of viral products, the resurrection of ERV in certain immunodeficiencies highlights the non-redundant roles of the host immune system. In particular, deficiencies in B cells and Toll-like receptor 7 (TLR7) signaling result in the emergence of infectious ERV. In the C57BL/6 background, a single ecotropic MLV (eMLV) locus, Emv2, has the capacity to generate infectious virions. Although germline Emv2 encodes a defective pol gene and a N-tropic capsid that is targeted by host restriction factor Fvl^{b}, this locus can recombine with other xenotropic MLV (xMLV) loci during integration into host cells, which restores the inherent defects in Emv2 to give rise to ERV viremia. Active transposition and de novo integration events of infectious ERV can cause insertional mutagenesis of host genes, LTR-mediated abnormal oncogene expression, and recombination-induced chromosomal aberrations that ultimately lead to development of lymphoma and morbidity.

Surprisingly, neither T cells nor major histocompatibility complex (MHC) class II presentation are required to prevent ERV emergence, suggesting that control of ERV is mediated through a T cell-independent (TI) B cell response. In addition, spontaneous ERV emergence is not observed in activation-induced cytidine deaminase (AID)—deficient mice or in immune-competent pups born to ERV viremic recombination-activating gene 1 (Ragl)—deficient breeders. This indicates that the fetal immune repertoire and germline-restricted B cell receptors (BCR) are sufficient to confer protection. However, it remains unclear how the fetal germline B cell repertoire mediates the blockade of ERV emergence to prevent subsequent damage to the host.

A major class of TI antibodies are the natural antibodies present in naïve mice prior to infection or immunization. Natural antibodies are produced by B-1 cells that reside in the peritoneal and pleural cavities, and marginal zone B (MZB) cells in the spleen. Generated from precursors in the fetal liver, B-1 cells possess inherent reactivity to self-antigens, upon which their BCR are positively selected during fetal and neonatal hematopoiesis, and to shared structures on foreign antigens. B-1 cells that are reactive to pathogen-associated antigens mediate a rapid TI response and generate high quantities of Immunoglobulin M (IgM) and IgG3, functioning as the first line of defense against infections. It has also been demonstrated that natural antiviral antibodies, including those against closely related retroviruses, are found in the sera of pre-immune mice. Where these antibodies arise and how they contribute to host anti-retroviral immunity is unknown.

The present study investigated the requirement of natural IgM and downstream effector molecules in the blockade of ERV emergence. The present study identified B-1 cells as the major source of natural anti-ERV antibodies through the development of a stringent antigen-baiting strategy. Single-cell RNA sequencing (scRNA-seq) and immune repertoire profiling of ERV-reactive B-1 cells were performed, and show that glycan-reactive antibodies are responsible for recognition of ERV through terminal N-acetylglucosamine (GlcNAc). The present study demonstrated that elevated secretion of ERV-reactive antibodies was induced by innate immune stimuli, such as lipopolysaccharide (LPS) and resiquimod (R848). Moreover, these antibodies were found to bind to a broad range of viral glycoproteins and enveloped viral surfaces through the recognition of a conserved terminal GlcNAc epitope. Collectively, these results reveal a broadly antiviral innate surveillance mechanism by natural antibodies.

### Example 2-2: Natural IgM Suppress ERV Reactivation through Complement Engagement

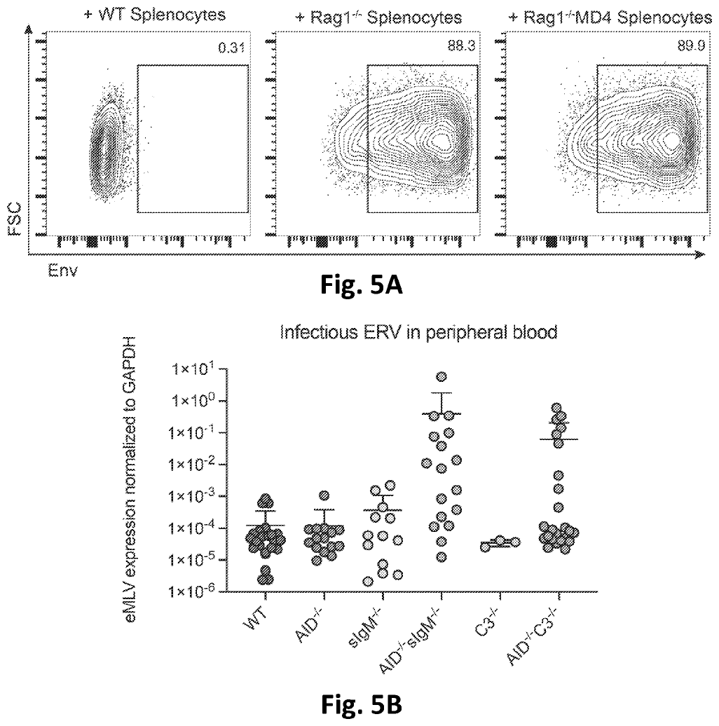

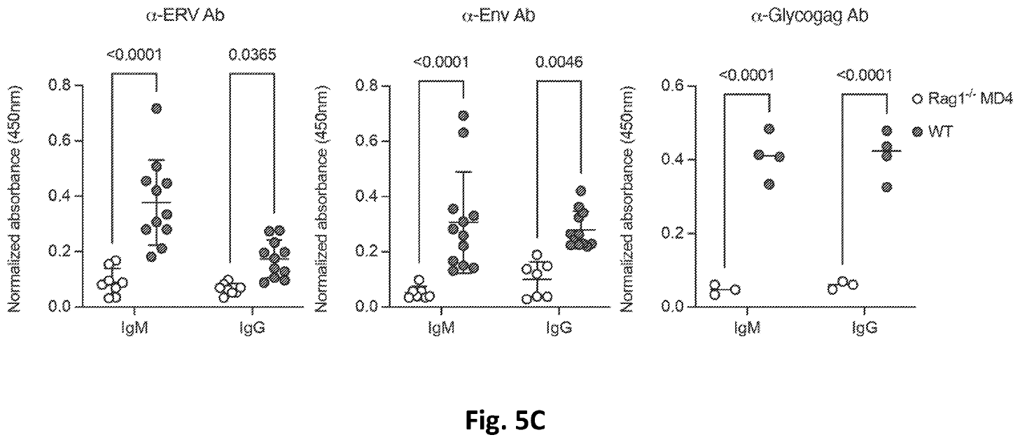

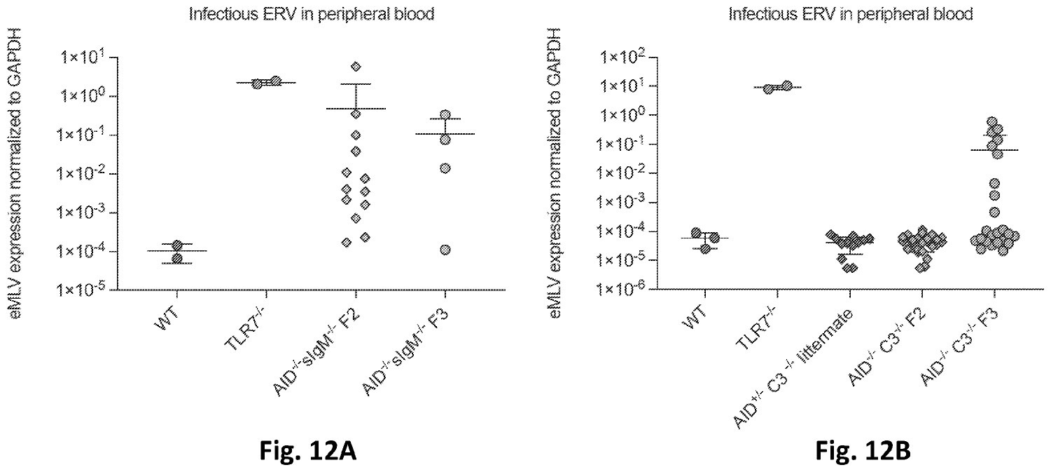

When the permissive DFJ8 cells, an avian fibroblast cell line that stably expresses the MLV entry receptor were co-cultured with splenocytes isolated from mice with deficiency in B and T cells (Ragl^{-/-}) or with a single invariant BCR to hen egg lysozyme (Rag1^{-/-} MD4 Tg), fully infectious ERV were isolated from splenocytes, and infected DFJ8 cells (FIG. 5A). Whether ERV control occurs through secreted antibodies or other B cell effector functions is unknown. In addition to the lack of ERV emergence in AID^{-/-} (Aicda^{-/-}) strain, ERV emergence was not observed in mice that are unable to secrete IgM due to a deletion of the secreted form of Igμ chain (sIgM^{-/-}), suggesting that either natural IgM without AID induced somatic hypermutation (SHM) or class-switched isotypes are independently sufficient to suppress ERV (FIG. 5B). To determine whether secreted antibodies are required for inhibiting ERV emergence, the present study next generated homozygous AID^{-/-}sIgM^{-/-} mice. Since the emergence of infectious ERV requires several rare recombination events to restore the replication defects in Emv2, ERV emergence was first observed in the second generation (F2) of homozygous knockout breeding (FIGS. 5B and 12A). The emergence of ERV in AID^{-/-} sIgM^{-/-} mice indicates the role for secreted IgM in preventing ERV emergence from mice with germline-restricted B cell repertoire.

Natural IgM in AID^{-/-} mice represents germline sequences that have not undergone SHM-mediated affinity maturation and therefore mediate low-affinity interactions with self-and foreign antigens. Complement was shown to be required for clearance of apoptotic cells and for bacterial and viral defense through natural antibodies. The complement system is an innate immune effector that induces direct killing or opsonizing of pathogens. Having multimeric structure, IgM is especially efficient in initiation of classical complement pathway upon antigen recognition through C1q deposition on bound IgM. The present study therefore sought to investigate the requirement for complement in ERV suppression by using mice deficient in complement pathway (C3^{-/-}) to generate AID^{-/-} C3^{-/-} mice. While ERV viremia did not manifest in C3^{-/-} mice, the present study observed ERV emergence by the third generation (F3) of AID^{-/-} C3^{-/-} homozygotes (FIGS. 5B and 12B), which demonstrated the indispensable role of complement in natural antibody-mediated control of ERV.

Having identified the mechanism by which secreted natural IgM confer host protection to ERV viremia, the present study next examined ERV-reactive antibodies in the serum of mice using ERV particles-coated enzyme-linked immunosorbent assay (ELISA).

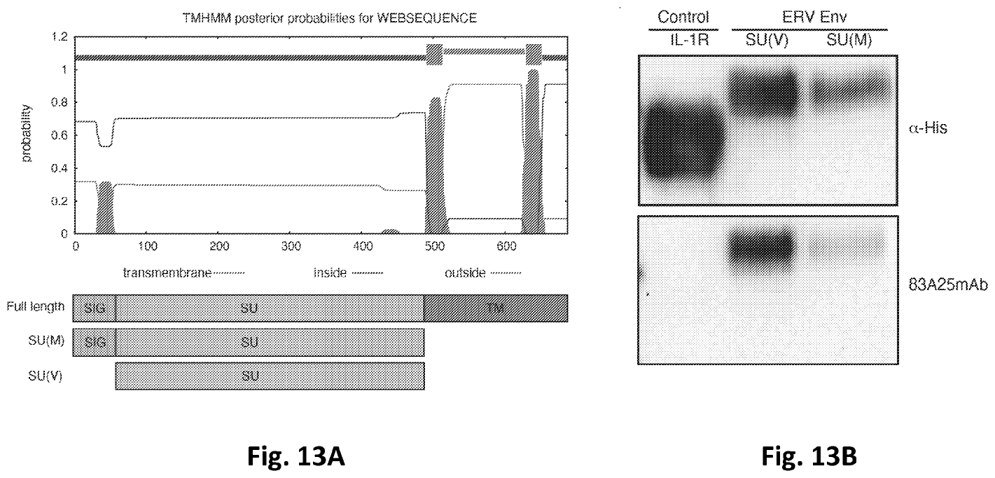

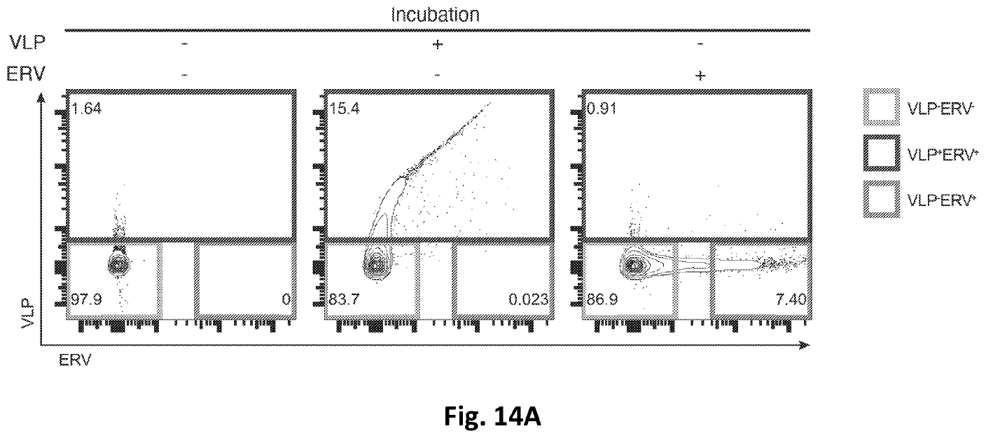

Anti-ERV antibodies were detected in the serum of wild-type (WT) adult C57BL/6 mice prior to any deliberate immunization (FIG. 5C). To rule out non-specific antibody reactivity to antigens derived from the DFJ8 cells in which the ERV particles were produced, the present study generated a recombinant ERV envelope (Env) surface subunit (SU) that retained antigenicity to the rat anti-ERV Env monoclonal antibody (mAb) 83A25 (FIGS. 13A-13B), and the glycosylated form of Gag protein (Glycogag). Env and Glycogag are considered to be the two major extracellular antigenic determinants that ERV-reactive antibodies recognize. Similarly, the present study detected anti-Env and anti-Glycogag antibodies in the sera of naïve WT mice (FIG. 5C). These results collectively show that germline-encoded ERV⁻ reactive natural IgM are present in the sera of naïve mice, and in their absence, ERV reactivation ensues.

### Example 2-3: ERV-baiting Strategy Identifies ERV-reactive B Cells Enriched in Peritoneal B-1 Compartment

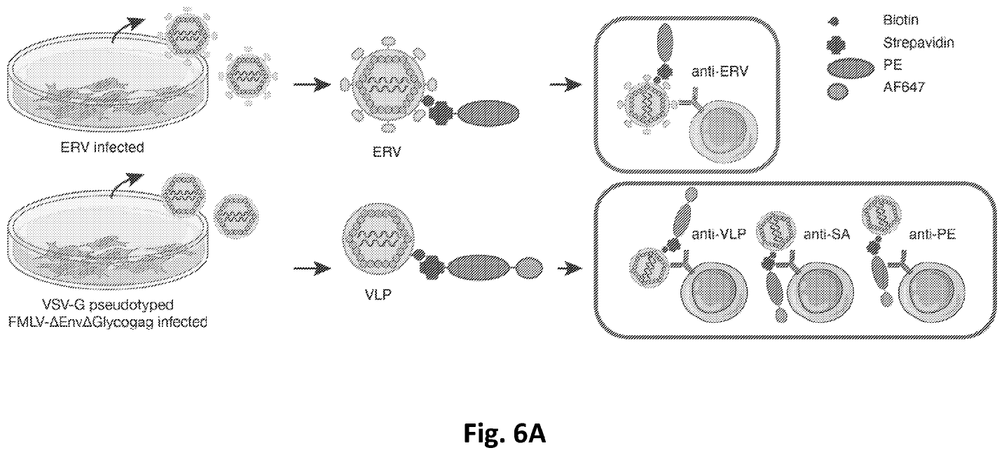

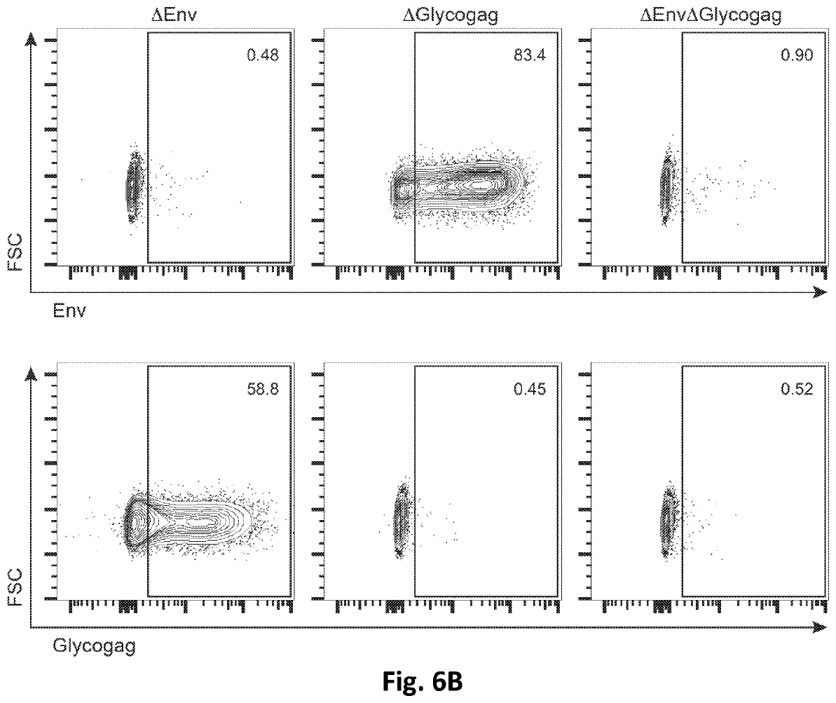

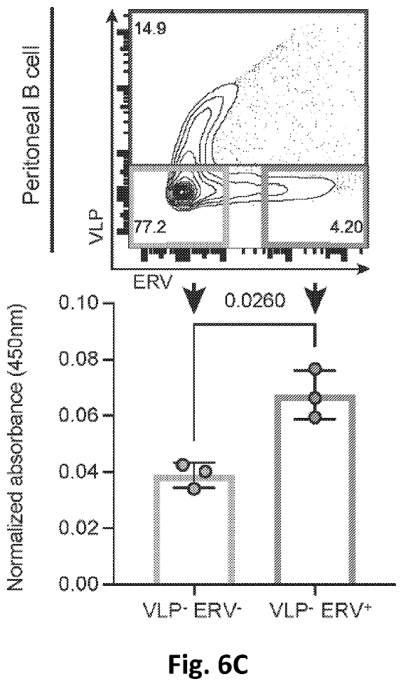

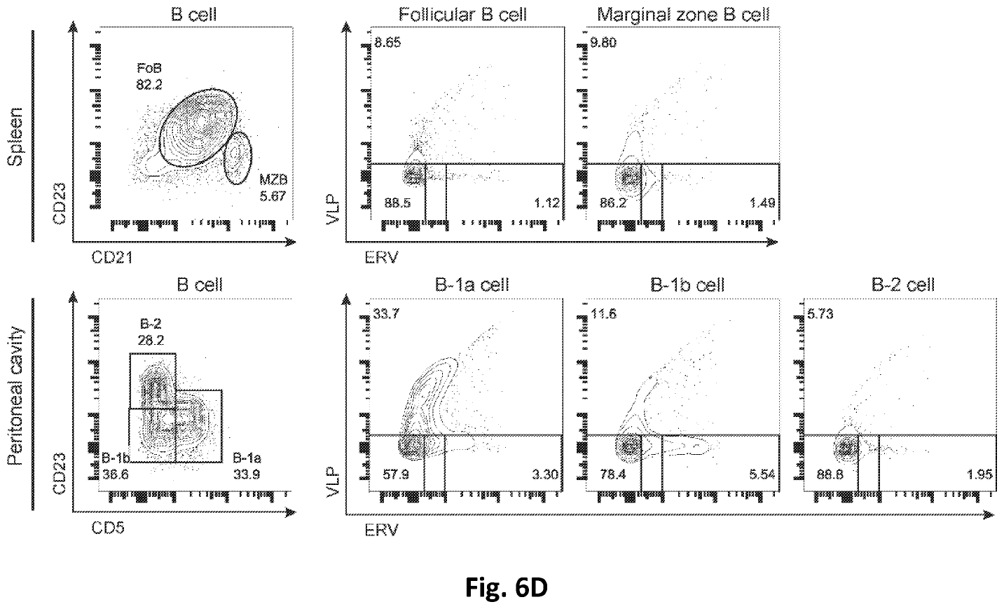

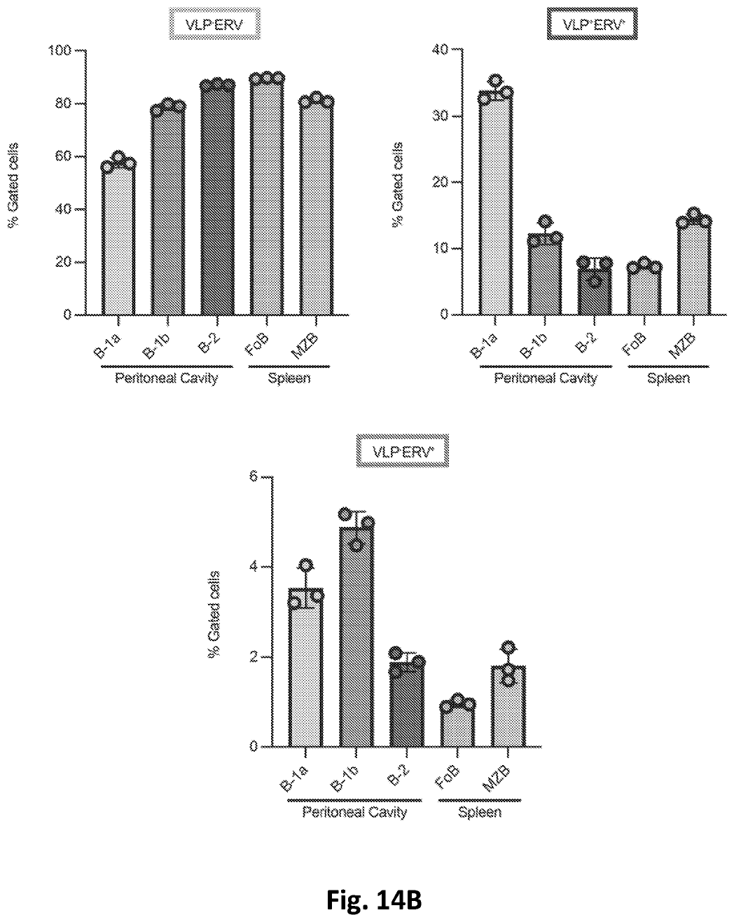

To identify the source of ERV-reactive antibodies, the present study developed an antigen-baiting assay to directly detect ERV-reactive B cells. ERV particles were purified from a single colony of ERV infected DFJ8 cells then was biotinylated for tagging with streptavidin R-phycoerythrin (SA-PE) (FIG. 6A). To resolve bona fide ERV-reactive B cells against high non-specific background mediated by naïve BCR recognizing biotin, SA, PE, and cell membrane constituent epitopes present on enveloped retroviruses, the present study generated a control-bait by producing MLV virus-like particles (VLP) lacking the two antigenic determinants Env and Glycogag in DFJ8 cells (FIG. 6B). The VLP were biotinylated and tagged with PE conjugated to Alexa Fluor 647 (AF647) dye (FIG. 6A). Thus, any B cells reactive to non-specific epitopes would emit AF647 signal in additional to PE signal in flow cytometry analysis (FIGS. 6C and 14A). As a validation of the ERV-baiting strategy, fluorescence-activated cell sorting (FACS)—isolated VLP⁻ERV⁺(AF674⁻PE^{Hi}) ERV⁻ reactive B cells secreted increased anti-Env IgM compared to VLP⁻ERV⁻ (AF674⁻PE⁻) non-ERV-reactive B cells (FIG. 6C).

The mouse spleen is primarily occupied by bone marrow-derived B-2 cells, with follicular B (FoB) cells located within the follicles and MZB cells located in the marginal zones. Whereas in the peritoneal and pleural cavities, two B-1 cell subsets, termed B-1a and B-1b cells which can be further distinguished based on CD5 expression, are far more abundant than conventional B-2 cells. The present study then used this ERV-baiting strategy to identify ERV-reactive B cells in different B cell compartments from peritoneal lavage and splenic cell suspension of naïve WT C57BL/6 mice. The cells were additionally stained for surface markers to identify FoB cells (CD5-CD21^{Mid}CD23^{Hi}) and MZB cells (CD5⁻CD21^{Hi}CD23^{Lo}) from splenic B cells (CD3⁻CD19⁺), and to identify B-1a cells (CD5⁺CD23^{-/-}), B-1b cells (CD5-CD23^{-/-}) and B-2 cells (CD5-CD23⁺) from peritoneal B cells (CD3⁻CD19⁺) (FIG. 6D). Among all B cell compartments examined, splenic FoB cells and peritoneal B-2 cells were the least reactive population, having the smallest percentage of cells bound to ERV or VLP. Although B-1a cells exhibited the greatest reactivity to VLP, ERV-reactive B cells were similarly enriched among peritoneal B-1a and B-1b cells, showing a 2-3-fold increase in the percentage of cells exclusively binding to ERV compared to other compartments. Indeed, within the peritoneal subset, the greatest frequency of ERV-reactive B cells (VLP⁻ERV⁺) was detected in the B-1b cell population (FIGS. 6D and 14B). Using the ERV-baiting strategy, the data indicate that ERV-reactive BCR are predominantly expressed by both peritoneal B-1 subsets, with a larger contribution from B-1b cells compared to B-1a subset.

### Example 2-4: Germline-restricted IgM Constitutes ERV-reactive Peritoneal B-1 BCR Repertoire

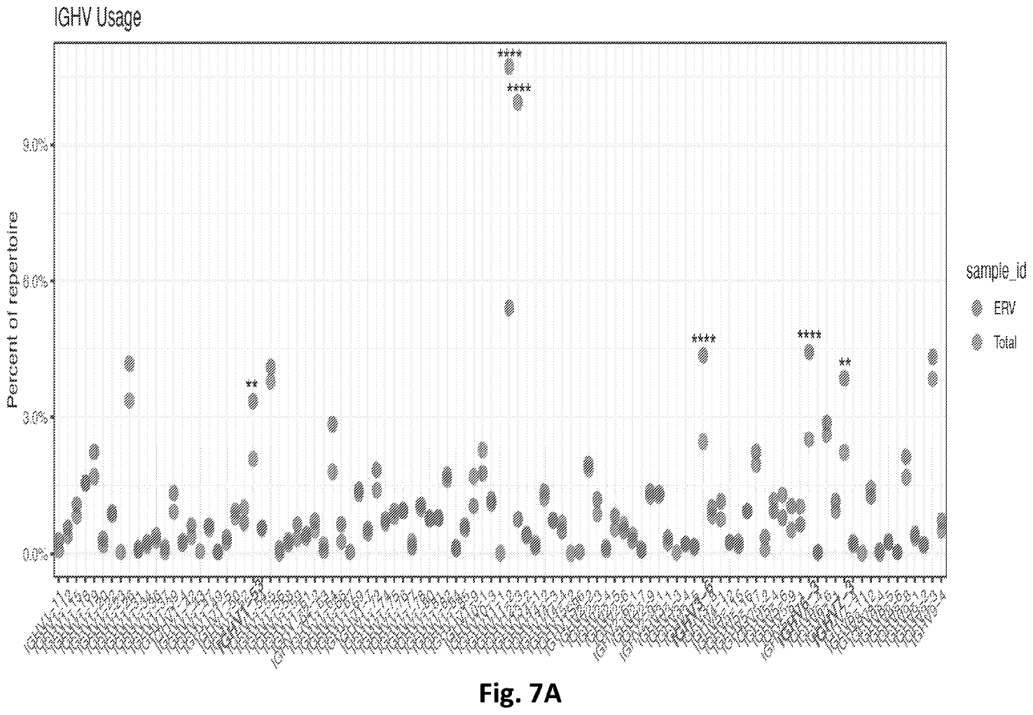

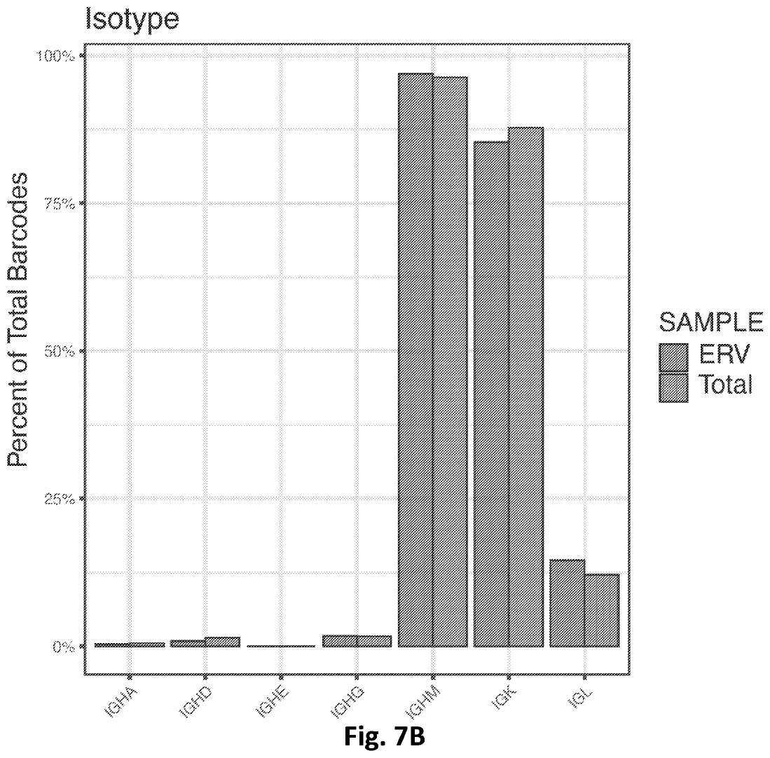

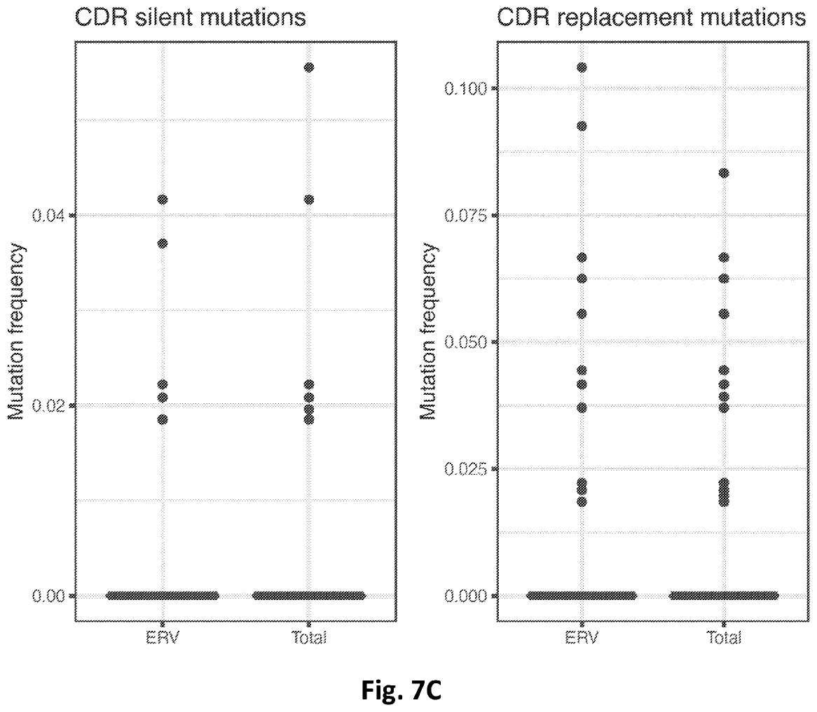

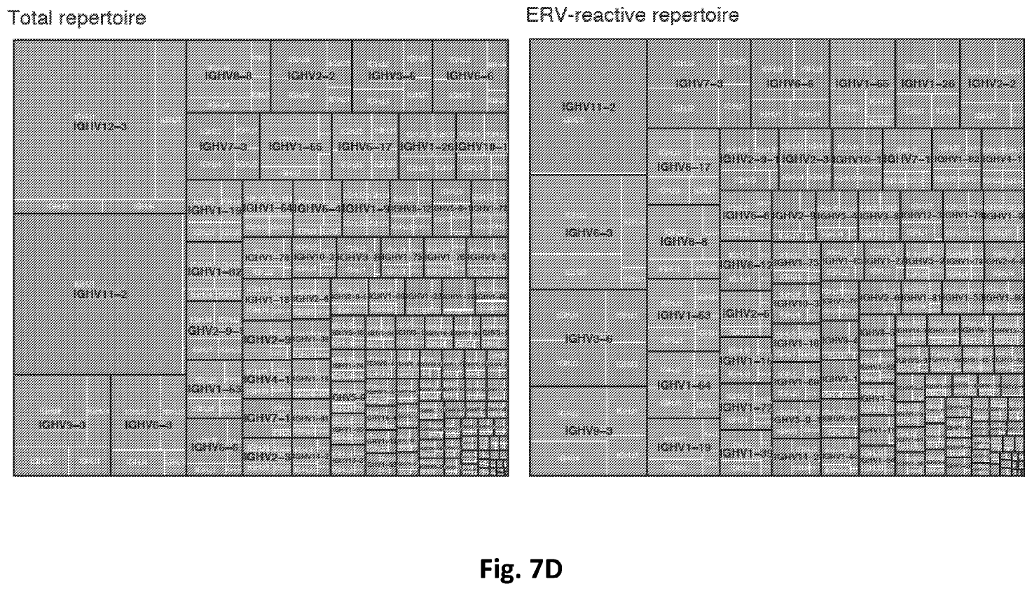

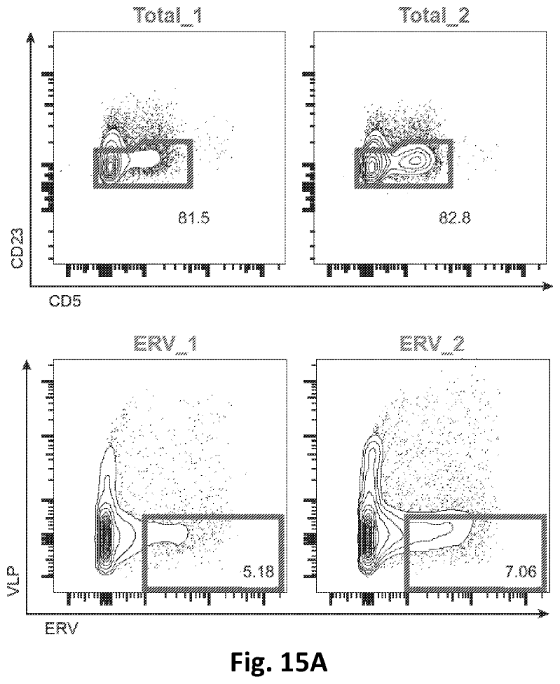

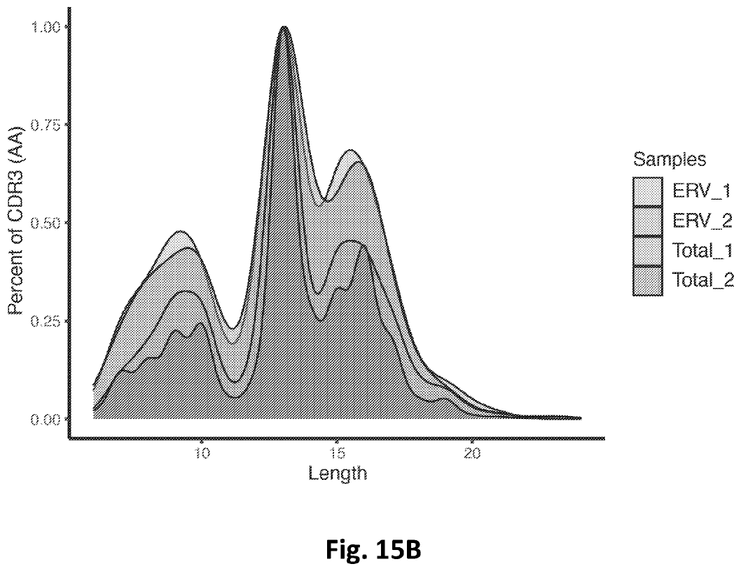

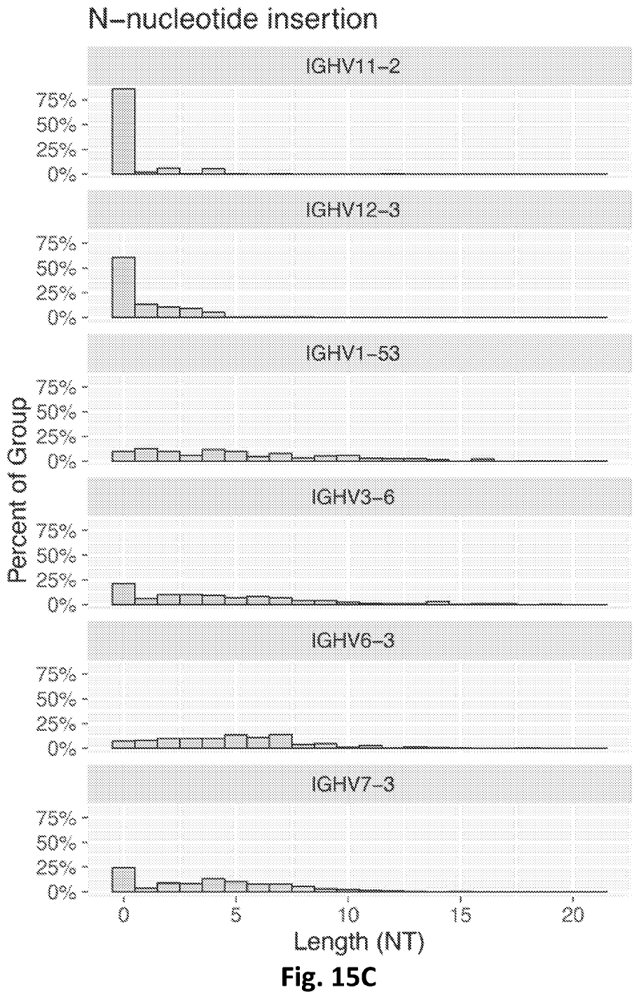

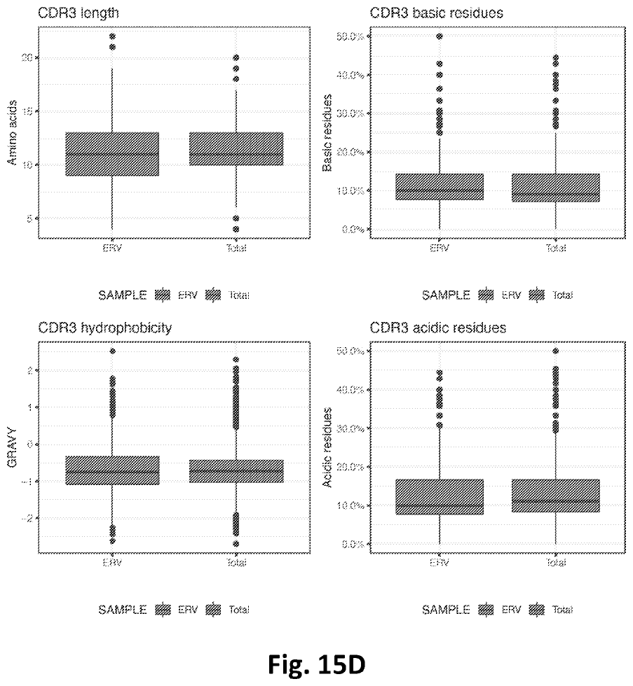

Having identified that ERV-reactive BCR are predominantly expressed by the peritoneal B-1 subsets, the present study next performed immune repertoire analysis of these B-1 cells. The antigen-baiting assay enabled accurate identification of bona fide ERV⁻ reactive B cells from the total naïve BCR repertoire. With this approach, ERV-reactive (VLP-ERV⁺) B-1 cells (CD3⁻CD19⁺CD23^{-/-}) and total peritoneal B-1 cells were sorted from naïve WT C57BL/6 mice for repertoire sequencing (FIG. 15A). The present study adopted the 10× Genomics Chromium Single Cell Immune Profiling platform to examine the paired immunoglobulin heavy chain (IgH) and light chain (IgL) sequences, in addition to the gene expression profiles with single-cell resolution (FIGS. 19-20). The immense diversity of the WT naïve BCR repertoire is obtained by somatic gene rearrangement of variable (V), diversity (D) and joining (J) segments that generate combinatorial and junctional diversity. Antibody diversity is further expanded by heavy chain and light chain pairing and somatic hypermutation (SHM). ERV-reactive BCR can be generated in the absence of AID^{-/-} induced SHM. In addition, the inherent poly- and auto-reactivities of B-1 BCR are largely determined by the selection of heavy chain V (IGHV/V_{H}) genes. Therefore, the present study first compared IGHV gene usage between ERV-reactive (3807 cells) and total B-1 cells (9662 cells) to characterize which V_{H} segments are enriched in the ERV-reactive repertoire and to identify putative ERV-reactive BCR. The present study identified four significantly enriched V_{H} genes: IGHV1-53, IGHV3-6, IGHV6-3 and IGHV7-3 (FIG. 7A). The two significantly depleted V_{H} genes in the ERV-reactive repertoire were IGHV 11-2 and IGHV12-3, which are known to encode anti-phosphatidylcholine (PtC) BCR and roughly comprise 5-15% of the peritoneal B-1 population. The vast majority of all sequences encoded for the Igμ constant region and thus class-switched antibodies were rarely observed in the ERV-reactive and total repertoires (FIG. 7B). Similarly, in the two repertoires, Igκ comprised ˜80% of sequenced light chains while Igλ were less frequent (FIG. 7B).

The present study did not observe significant differences in the rate of silent mutation or replacement mutation across predicted complementarity-determining regions (CDR) between ERV-reactive and total BCR repertoires (FIG. 7C). Indeed, both repertoires displayed no signs of somatic hypermutation, which is consistent with the notion that B-1 cells harbor germline-encoded BCR. BCR within the ERV-reactive repertoire exhibited similar average CDR3 length, percentage of basic and acidic residues, and CDR3 hydrophobicity when compared to BCR within the total repertoire (FIG. 15D). However, when looked at the length distribution of the CDR3 sequences, ERV-reactive BCR displayed a broader diversity of CDR3 length (FIG. 15B). The present study further examined the diversity of CDR3 by investigating J segments coupling to individual V_{H} genes. Significantly distinct from the PtC-reactive IGHV11-2 and IGHV12-3 that predominantly, if not exclusively, recombined with IGHJ1, putative ERV-reactive V_{H} genes did not show selection bias for any of the four JH segments in V(D)J recombination (FIG. 7D). The CDR3 diversity in ERV-reactive BCR is further contributed by non-templated nucleotide insertion at the V(D)J junctions (FIG. 15C), which is mediated by terminal deoxynucleotidyl transferase (TdT) induced after birth. This observation is indeed in concert with the findings that the largest percentage of ERV-reactive B cells is detected in B-1b subset, which is replenished by bone-marrow derived precursors and harbors greater diversity than does the B-1a compartment. In brief, the present study demonstrated that the ERV-specific BCR repertoire is predominantly comprised of germline restricted Igμ sequences with diversified CDR3 length and usage of all four JH genes.

### Example 2-5: ERV-reactive B-1 Cells Consist of Clones with Unique Gene Expression Patterns

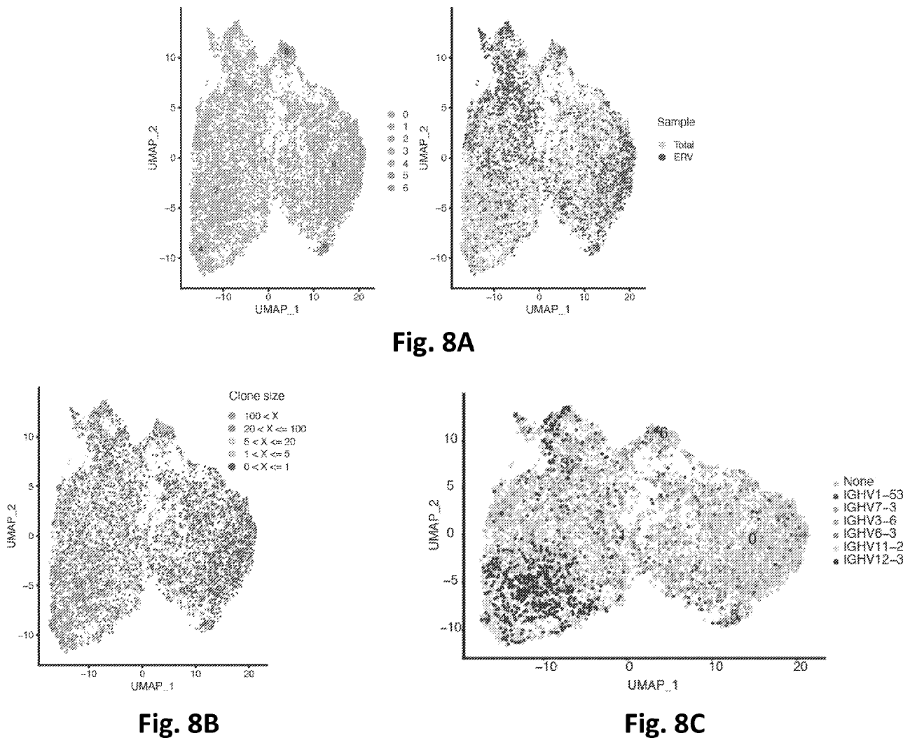

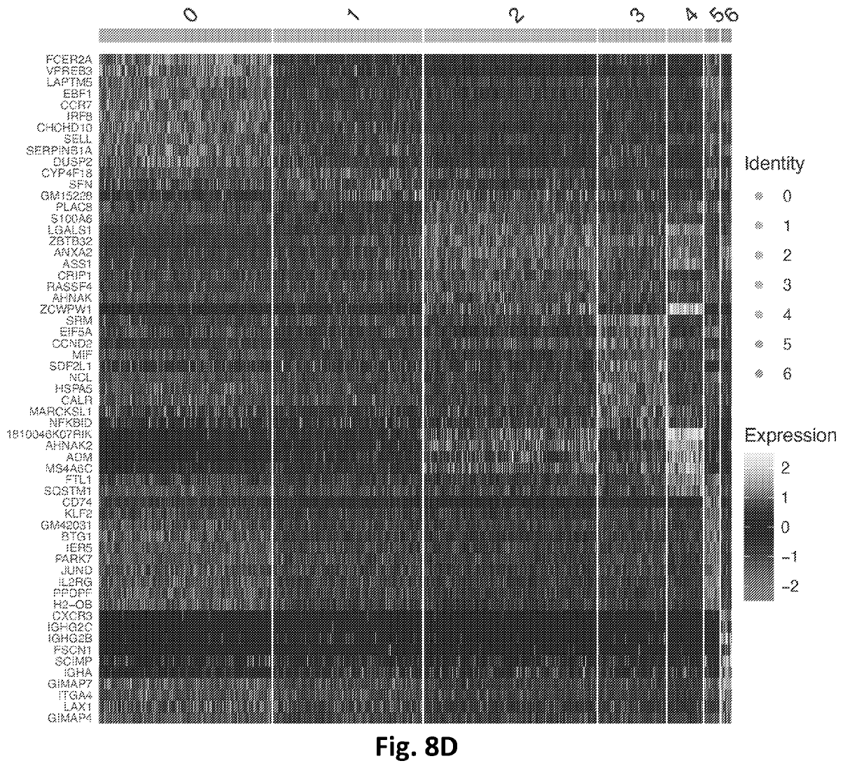

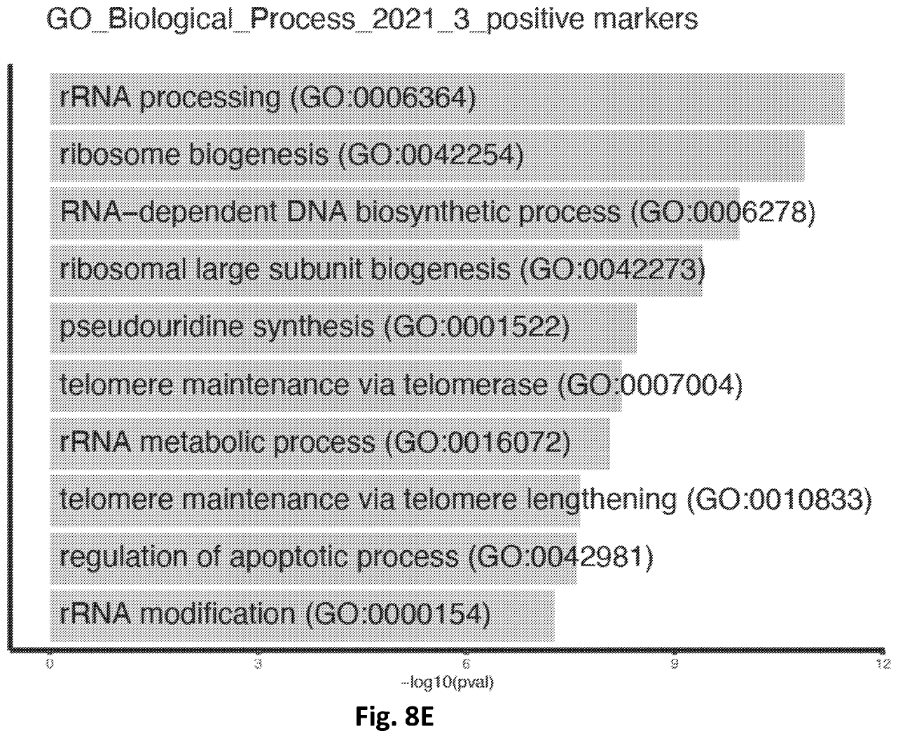

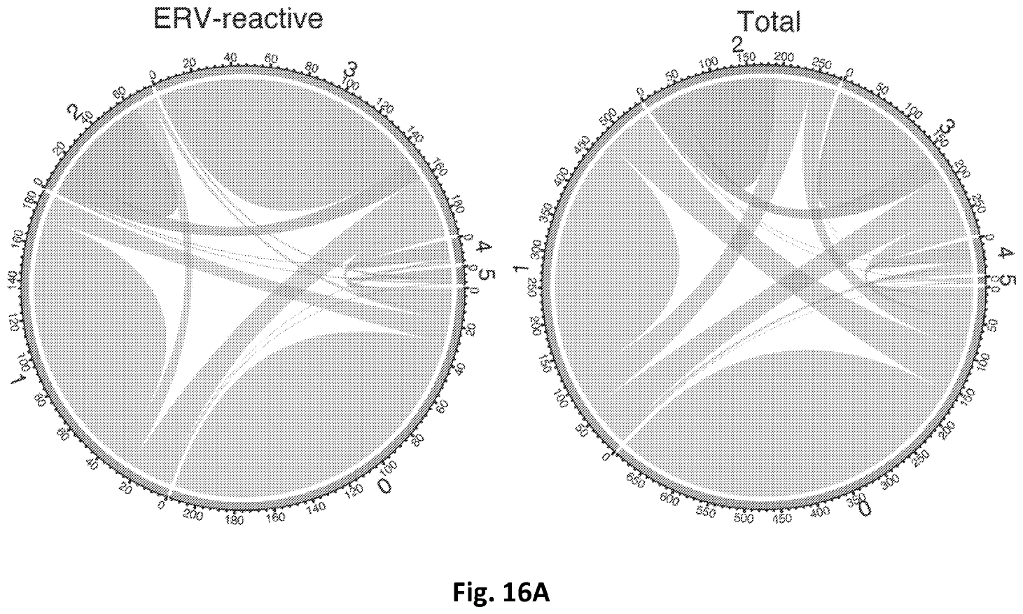

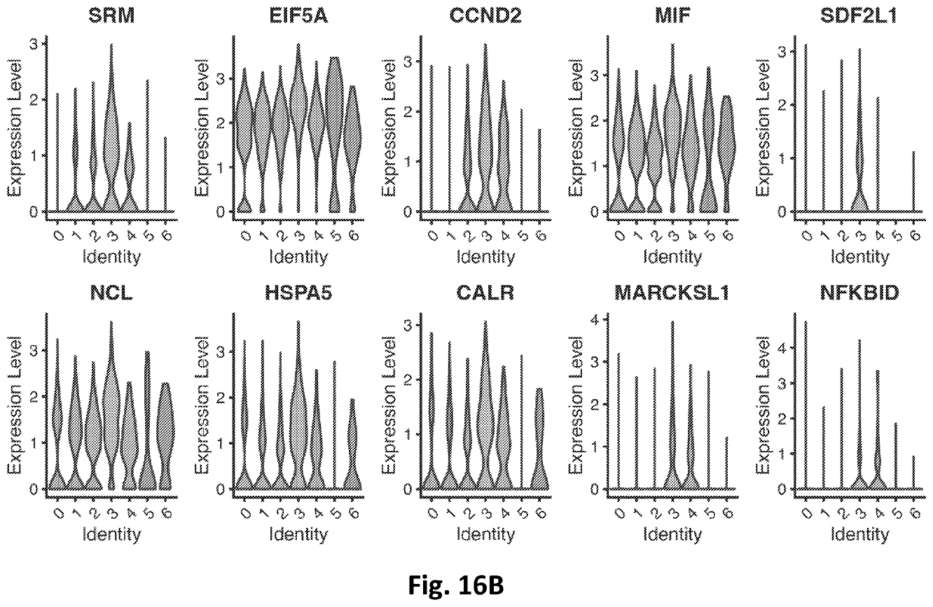

The present study performed single-cell transcriptomic profiling on ERV-reactive and total B-1 cells, for which the present study also profiled the BCR repertoire. Unbiased hierarchical clustering of 4003 ERV-reactive B-1 cells and 10231 total naïve B-1 cells showed a landscape of six clusters of phenotypically diverse B-1 cells (FIG. 8A). Because all sequenced cells are FACS-sorted peritoneal B-1 cells, a clear segregation of clusters was not obtained by dimension reduction using Uniform Manifold Approximation and Projection (UMAP). ERV-reactive cells are largely categorized into cluster 0 and cluster 3 (FIG. 8A). Percentage of cells assigned to cluster 3 significantly increased in ERV-reactive repertoire as compared to total naïve B-1 repertoire (FIG. 16A). Clonal lineage analysis revealed enrichment of clonally expanded medium-frequency- and large-frequency-clones in cluster 2 and cluster 3 (FIG. 8B). Notably, the ERV-baiting assay increased the percentage of clones in cluster 3 that did not share clonal relationship with other clusters (FIG. 16A), suggesting that ERV-reactive B cells are a clonally and phenotypically distinct population. To avoid the impact of highly expressed Ig genes associated with B cells in the clustering analysis based on the highly variable features, the present study removed gene transcripts that mapped to the Ig locus. By doing so, clustering of B cells would not be driven by Ig genes annotation. However, when the present study integrated gene expression profiles with BCR sequence analysis based on cell barcodes after clustering, it was observed that anti-PtC BCR expressing B cells are strongly segregated into cluster 2, and to a lesser extent, putative ERV-reactive V_{H} genes were enriched in cluster 3 (FIG. 8C), suggesting that both PtC-reactive BCR and ERV⁻ reactive BCR may be associated with distinct gene-expression profiles.

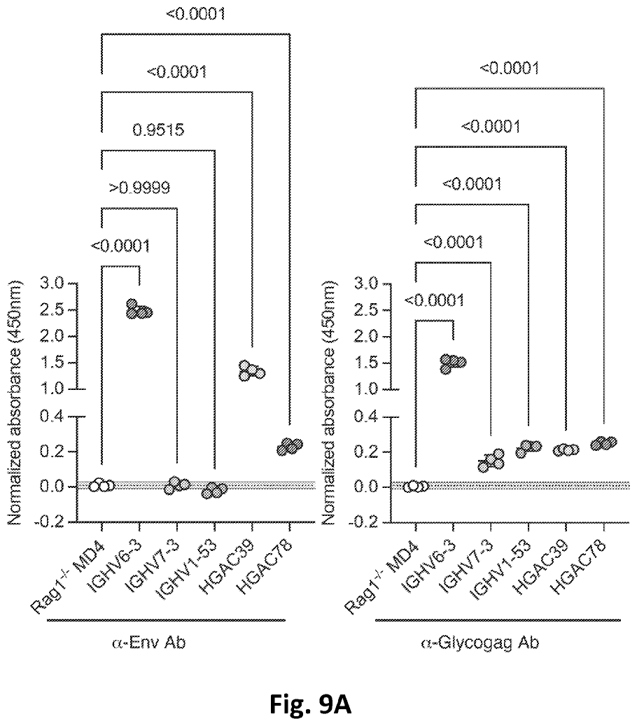

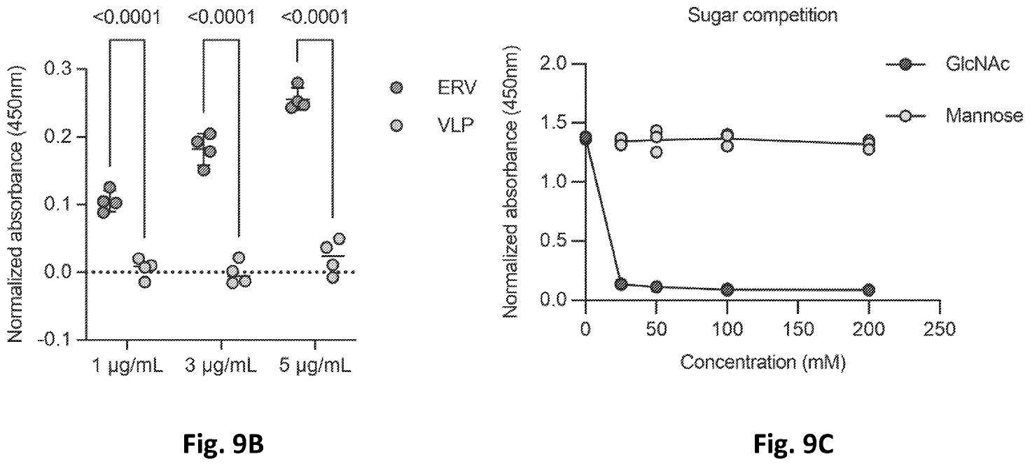

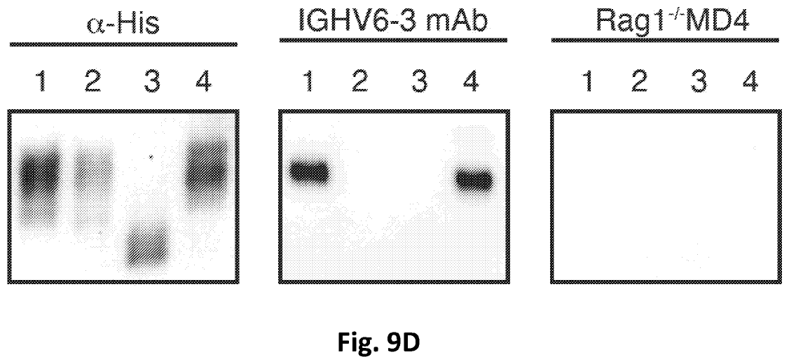

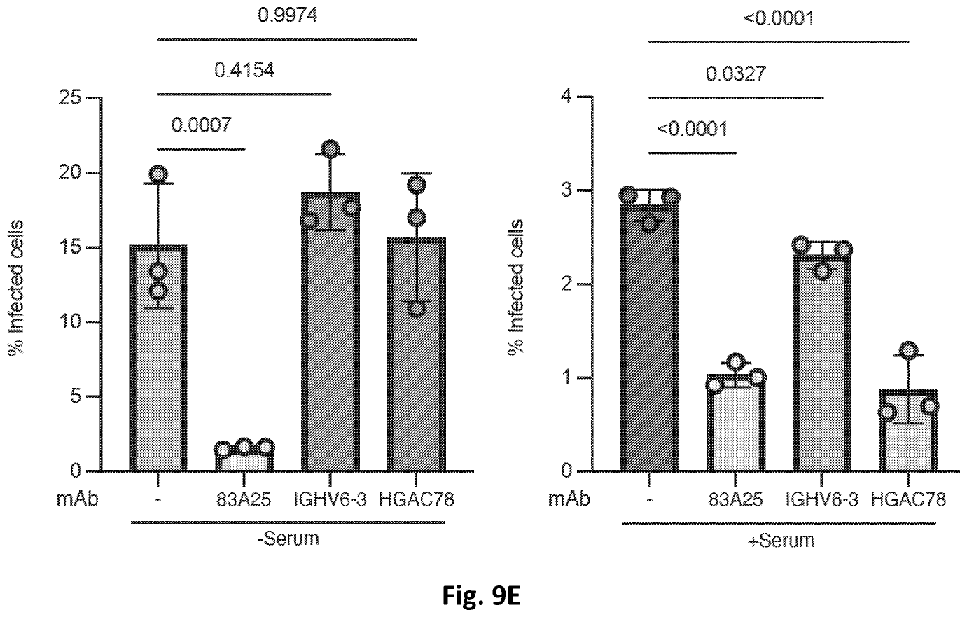

To further characterize different transcriptomic-defined clusters, the present study examined the differentially expressed genes (DEG) in all clusters and discovered a set of genes highly expressed in cluster 3 (FIG. 8D). Gene ontology (GO) analysis on markers of biological processes enriched in cluster 3 suggested anti-apoptotic and pro-survival features of this population (FIG. 9E). More specifically, in addition to Ccnd2 encoding cyclin D2 that is particularly essential for self-renewal of B-1 cell pools, the present study identified that spermidine synthase (Srm), eukaryotic translation initiation factor. 5A (elF5a), macrophage migration inhibitory factor (Mif) and heat shock protein family A member 5 (Hspa5) which promote cell proliferation or suppress apoptosis in lymphoid linages and/or cancer cells, were upregulated in cluster 3 (FIG. 16B). Taken together, these findings suggested that ERV⁻ specific B-1 cells possess distinct features consistent with promoting self-renewal and survival.

### Example 2-6: IGHV6-3 mAb Enhances Complement-dependent Neutralization of ERV through Recognition of Terminal GlcNAc

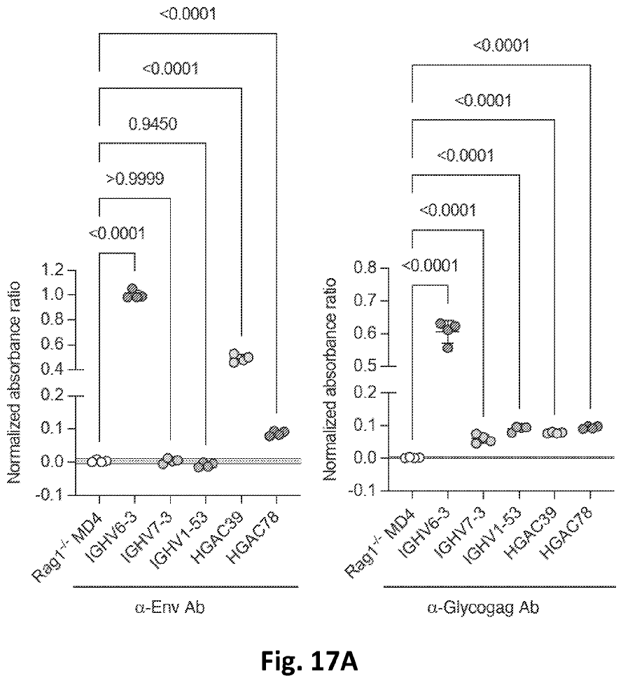

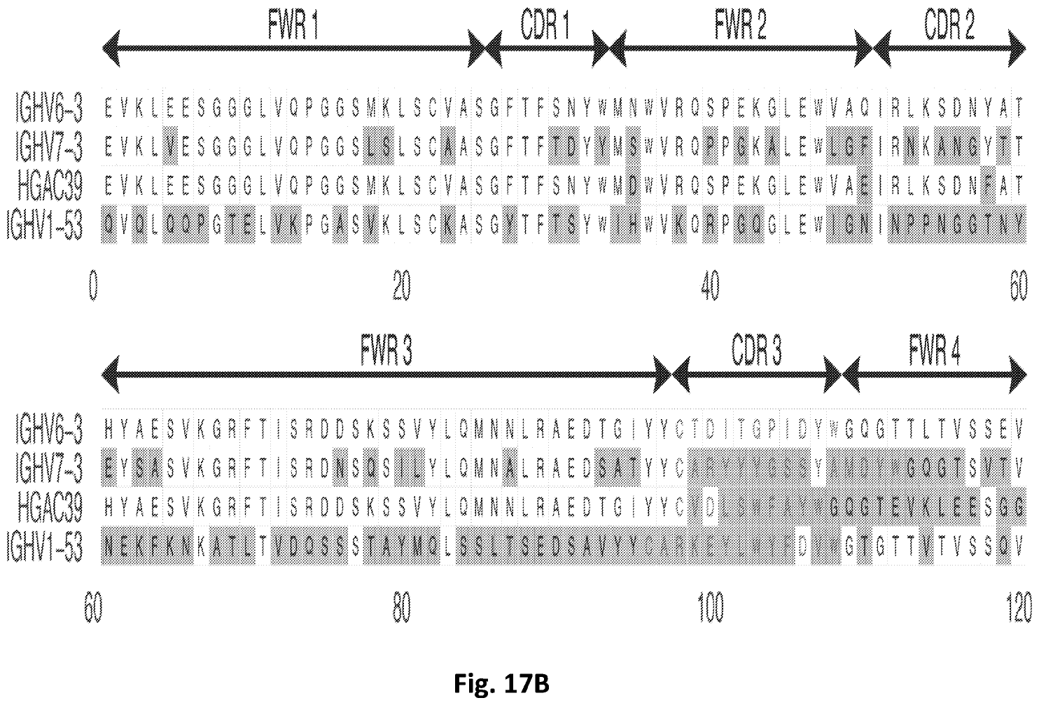

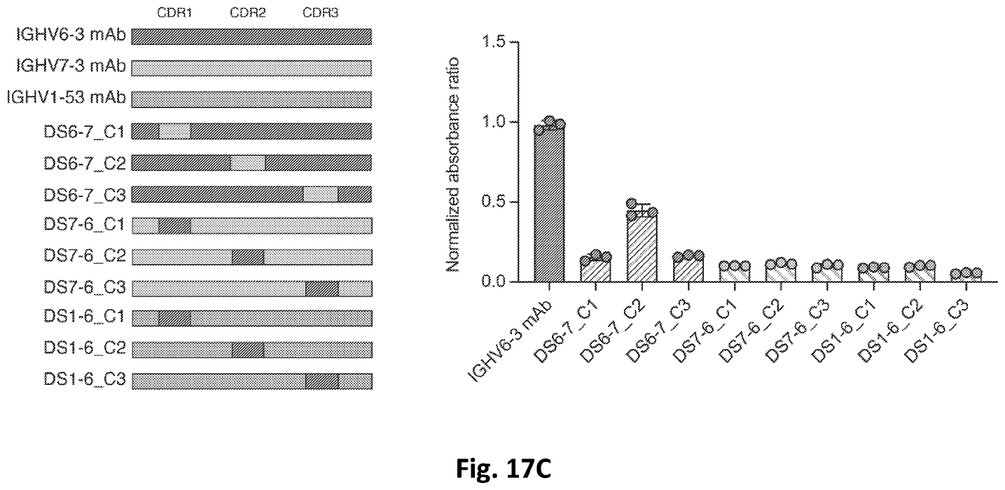

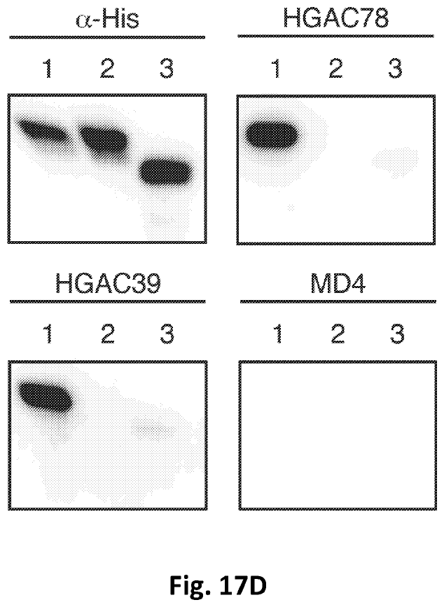

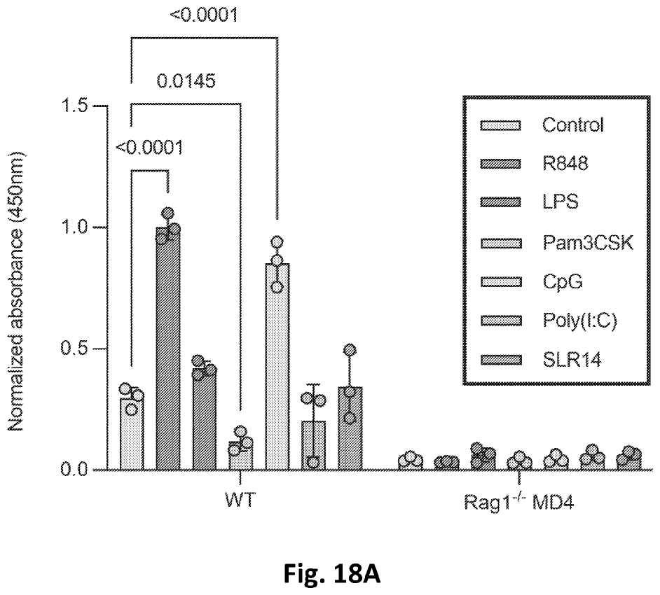

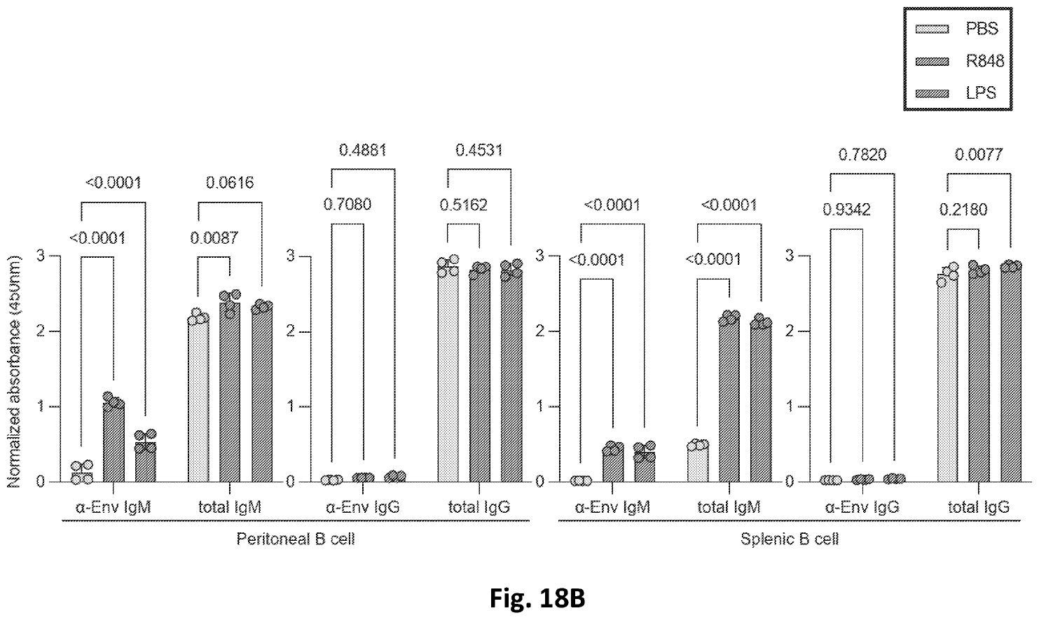

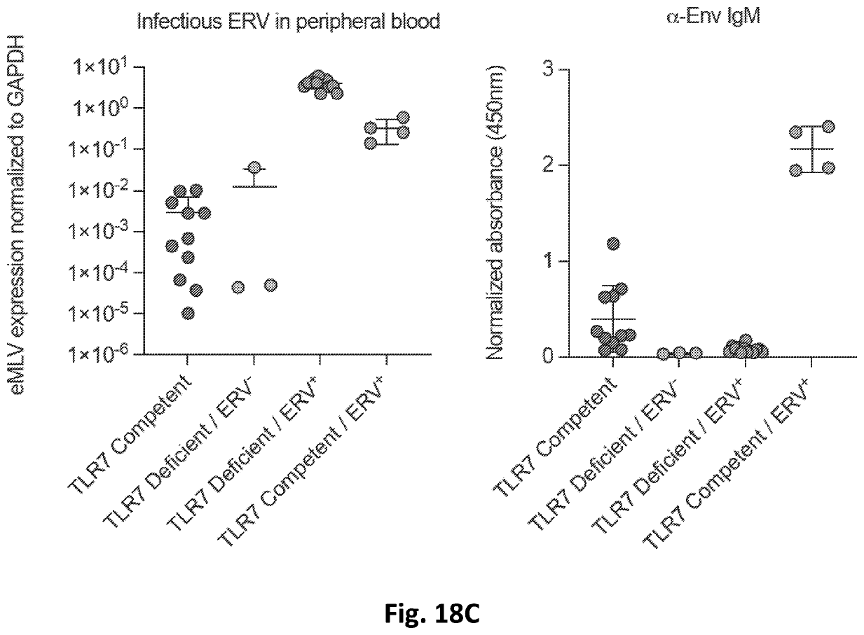

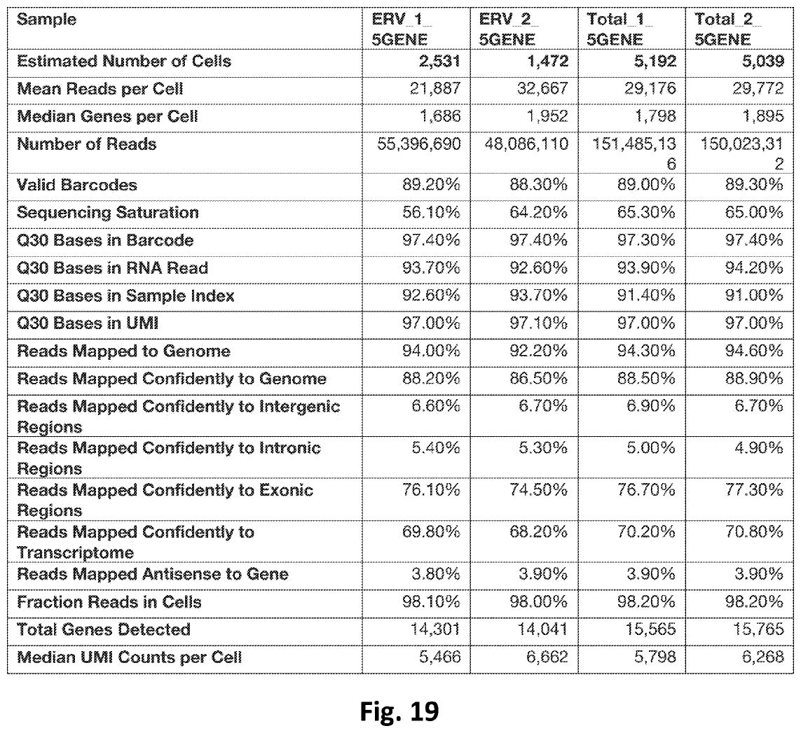

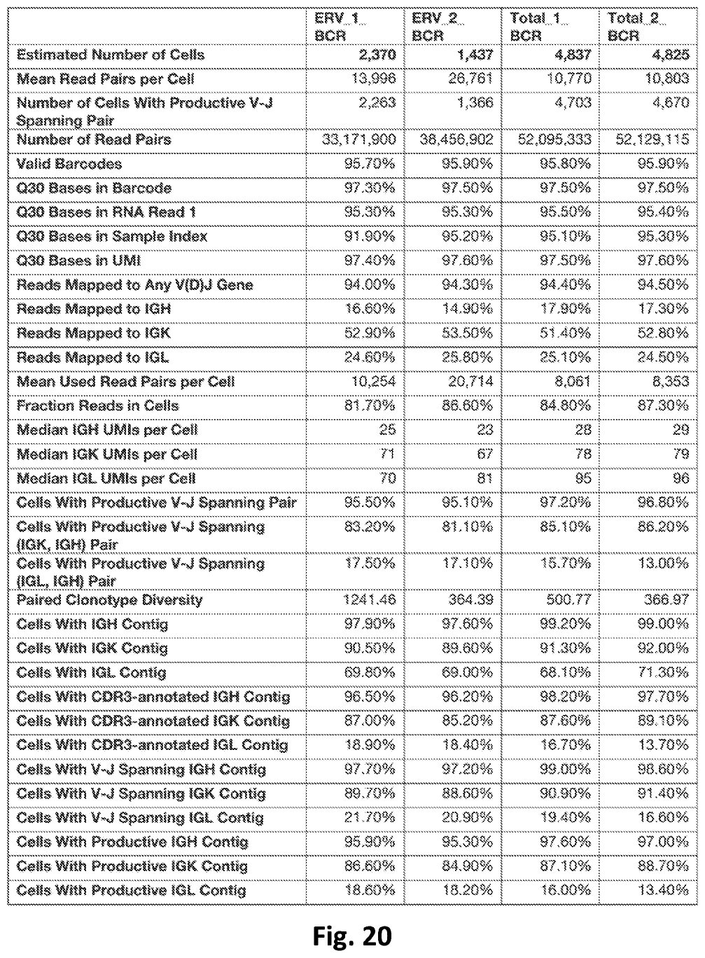

With the paired IgH and IgL sequences provided by single-cell immune profiling, the present study was able to generate recombinant monoclonal antibodies to validate these putative ERV-reactive BCRs. For each enriched V_{H} genes including IGHV1-53, IGHV3-6, IGHV6-3 and IGHV7-3, the present study identified the largest clone within the ERV⁻ reactive repertoire expressing those V_{H} genes, and generated a consensus sequence for IGHV1-53, IGHV3-6, IGHV6-3 and IGHV7-3 encoded IgH and their paired IgL based on the largest clones. These IgH and IgL variable regions were cloned into the mouse IgG2c and Igκ backbone vectors respectively to generate full-length antibodies. Except for IGHV3-6 sequences that failed to generate a full-length paired antibody (possibly due to inefficient pairing of the IgH and IgL), the present study obtained monoclonal antibodies (mAb) encoded by IGHV1-53, IGHV6-3 and IGHV7-3. The previously reported Lancefield group A carbohydrate (GAC)—reactive antibodies that are critical in protection against Streptococcus pyogenes infection, are also encoded by a IGHV6-3 rearranged BCR. The present study observed that the variable region of mAb HGAC39 (Cy3) and HGAC78 (Cp) which are generated from GAC-reactive hybridomas, shared ˜87% amino acids homology with the IGHV6-3 ERV-reactive BCR identified (FIG. 17B). The present study therefore tested the three mAb generated, in parallel with HGAC39 and HGAC78, for reactivity against ERV antigens. IGHV6-3 mAb reacted strongly to both Env and Glycogag, while HGAC mAb exhibited weaker reactivity to both antigens and IGHV1-53 mAb and IGHV7-3 mAb only weakly reacted with Glycogag (FIGS. 9A and 17A). Having identified IGHV6-3 mAb as having strong ERV reactivity, the present study further verified that it exclusively recognized ERV particles, but not VLP lacking Env and Glycogag antigens (FIG. 9B). To investigate which IGHV6-3 mAb domains are required for Env recognition, the present constructed domain-swapped antibodies by exchanging each individual CDRs between IGHV6-3 and IGHV7-3 or IGHV1-53 (FIG. 17C). While IGHV1-53 mAb did not share sequence homology with IGHV6-3 mAb, IGHV7-3 mAb are ˜63% identical to IGHV6-3 mAb across the variable region. Similar to IGHV6-3 clones, IGHV7-3 clones are induced by GAC immunization. However, the framework provided by IGHV6-3 appeared to be involved in Env recognition, indicated by the loss of reactivity that occurs when CDRs from IGHV6-3 are grafted onto either IGHV1-53 or IGHV7-3 FWR (FIG. 17C).

The epitope for GAC recognition by HGAC mAb is N-acetylglucosamine (GlcNAc) side chains on bacterial polysaccharide. Given the cross-reactivity of HGAC mAb to ERV Env, it was hypothesized that IGHV6-3 mAb with sequence similarity to HGAC mAb may recognize a similar epitope on ERV. Supporting this hypothesis, IGHV6-3 mAb failed to bind to ERV Env when being saturated with free GlcNAc that can compete away GlcNAc binding site (FIG. 9C). The present study then took a step forward to investigate what types of GlcNAc residues mediate immune recognition by IGHV6-3 mAb. The array of multiple GlcNAc residues on the non-reducing end of GAC O-linked glycans are uniformly expressed in all group A streptococcus serotypes. However, these residues are absent on mammalian cells and are therefore targeted by the host immune system. Although structurally distinct from bacterial lipopolysaccharides, MLV Env gene product contains six to eight N-linked oligosaccharides and at least one O-linked glycan, all located on the external gp70 subunit. Enzymatic treatments were employed with 1) D-N-acetylglucosaminidase S to exclusively remove terminal non-reducing GlcNAc on ERV Env, 2) PNGase F to cleave between the inner most GlcNAc and asparagine (Asn) and thus remove all N-linked glycans, and 3) a combination of O-glycosidase and neuraminidase to remove O-linked glycans. Interestingly, removing terminal non-reducing GlcNAc is sufficient to abolish IGHV6-3 mAb reactivity, and these terminal GlcNAc are present on N-linked glycans (FIG. 9D). The cross-reactivity of HGAC mAb to ERV Env was also found to be mediated by terminal GlcNAc (FIG. 17D).

After identifying the reactivity of IGHV6-3 mAb for ERV terminal GlcNAc, the present study sought to validate this in a functional assay. As described in a previous section, it was demonstrated that natural antibodies in pre-immune mice elicit protection against ERV emergence by engaging complement cascade. The present study therefore tested whether IGHV6-3 mAb could inhibit ERV infection of DFJ8 cells in the presence of complement. In the absence of serum, although ERV-specific rat mAb 83A25 efficiently neutralized ERV, neither IGHV6-3 mAb nor HGAC78 mAb reduced ERV infection. However, in the presence of Ragl MD4 Tg serum that contains infectious ERV and complement proteins but no ERV-reactive antibodies, IGHV6-3 (Cy2c) mAb, and HGAC78 (Cp) mAb to a greater extent, inhibited ERV infection, which suggests that these antibodies exerted ERV control in a complement-dependent manner, with efficiency correlating with the capacity of each isotype to fix complement (FIG. 9E). Collectively, the results demonstrated that recombinant mAb encoded by IGHV6-3 recognizes ERV through terminal GlcNAc residues and recruit complement to inhibit infection.

### Example 2-7: Secretion of ERV-reactive Antibodies is Driven by Innate Sensor Stimulation

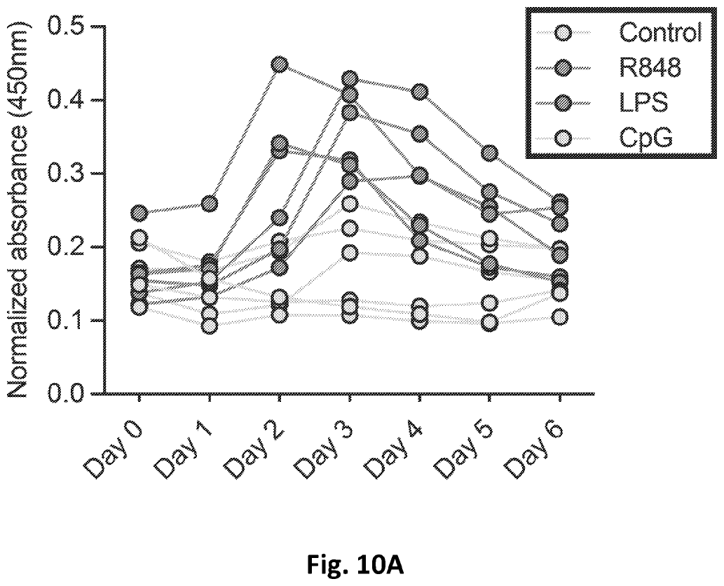

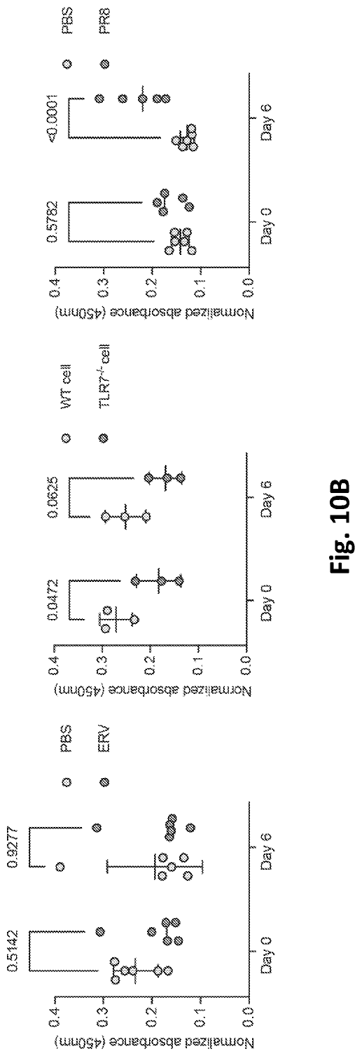

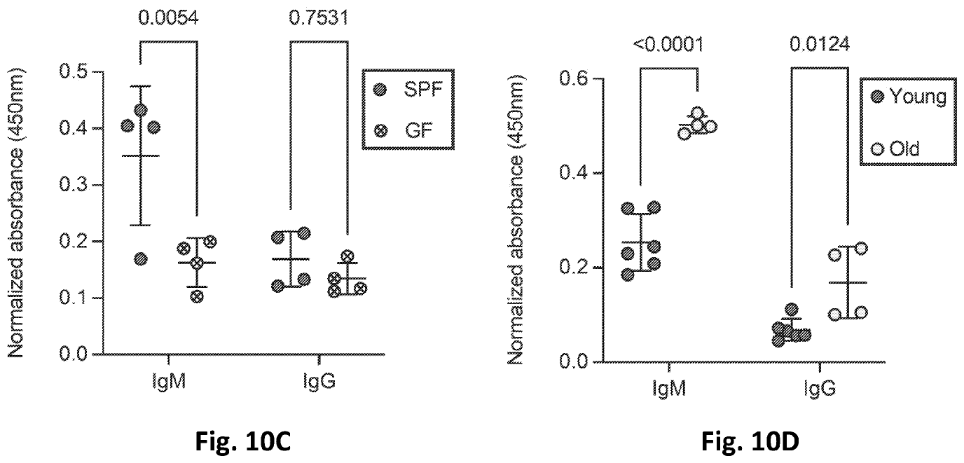

The ERV viremic Tlr7^{-/-} mice demonstrated the requirement of nucleic acid sensing pathway for ERV control. Moreover, earlier studies have uncovered the role of TLR4, TLR7 and TLR9 in BCR-independent and dependent responses that allow rapid induction of B-1 cell functions, including egress of B-1 cells from the body cavities to the spleen, and differentiation into antibody secreting cells. Therefore, to dissect the role of different innate signaling sensing pathways in the induction of anti-ERV natural antibodies, peritoneal B cells from naïve mice were isolated and stimulated in vitro with synthetic ligands for different innate sensors. Anti-Env IgM was significantly induced upon TLR7 and TLR9 activation with R848 and CpG DNA respectively. This was not observed with Pam3CSK activation of TLR2, Poly(I.C) activation of TLR3, retinoic acid-inducible gene I (RIG-I) and melanoma differentiation-associated protein 5 (MDAS), or stem-loop RNA 14 (SLR14) activation of RIG-I. LPS activation of TLR4 slightly enhanced anti-Env IgM secretion in vitro (FIG. 18A). In naïve mice, intra-peritoneal (i.p.) injection of TLR7 and TLR4 agonists rapidly induced serum anti-Env IgM as early as day 2 post-injection, while mice that received TLR9 agonist i.p. displayed a minimal response (FIG. 10A). It was further confirmed that the induction of anti-Env IgM secretion by TLR ligands was not restricted to B cells residing in the peritoneal cavity, as it was also observed in isolated splenic B cells, although to a lower level (FIG. 18B).

The present study further ruled out the requirement of cognate antigen encounter for the induction of anti-Env IgM, as it was not observed in mice receiving i.p. challenge with infectious ERV particles nor splenic T cells from Tlr7^{-/-} mice that highly expressed ERV Env on cell surface (FIG. 10B). In contrast, mice intranasally (i.n.) infected with influenza A strain A/Puerto Rico/8/34 (H1N1; also known as PR8), upregulated anti-Env IgM secretion, suggesting that unrelated pathogen challenges that efficiently activates TLR7 through single-stranded RNA without involving cognate antigen recognition are sufficient for induction of anti-Env IgM (FIG. 10B). Although the present study was not able to establish acute ERV infection with reactivated ERV particles in mice, Tlr7^{+/-} female breeders were crossed with Tlr7^{-/y} male breeders to obtain TLR7-deficient F1 offspring and monitored de novo ERV emergence in the absence of vertical transmission of ERV from female breeders. As these TLR7-deficient strains were generated on the C57BL/6N background, which possess elevated levels of non-ecotropic ERV (NEERV) transcripts, Emv2 recombination occurred at an accelerated frequency. It was observed ERV emergence in 13/16 F1 offspring (FIG. 18C). Compared to TLR7-competent littermates with variable levels of natural ERV-reactive antibodies, none of the TLR7-deficient offspring, whither with or without ERV viremia, produced detectable anti-Env IgM. This is in stark contrast to TLR7-competent mice that suffer from ERV viremia (AID^{-/-} C3 ^{-/-}) and significantly upregulate anti-Env IgM, indicating that the TLR7 signaling pathway is required for induction of natural antibodies against ERV (FIG. 18C).

Apart from a direct impact on the induction of natural ERV-reactive antibodies, TLR signaling may interplay with BCR signaling that drives clonal expansion of ERV⁻ reactive B-1 cell clones, affecting antibody levels at steady state, as hinted by the near-undetectable level of anti-Env IgM in TLR7-deficient mice, regardless of ERV viremia (FIG. 18C). Although there is no ERV-free mouse models, a previous study has demonstrated that neonatal exposure to the microbiota drives clonal expansion of IGHV6-3 encoded clones reactive to GAC. Indeed, it was observed that anti-Env IgM was reduced in the serum of germ-free mice at steady state compared to specific pathogen-free (SPF) mice, which is unsurprising given the overlapping epitopes of GAC and ERV that these glycan-specific antibodies recognize (FIG. 10C). Both BCR- and TLR-mediated signaling are integral triggers in directing positive selection of B-1 cell clones reactive to microbiome or self-antigens. Additionally, it was observed that serum anti-Env IgM levels significantly increased with age (FIG. 10D). Given the short half-life of IgM, the observed phenotype reflects constant shaping of the ERV-reactive clonal repertoire rather than an accumulated effect. Thus, these data underscored the function of innate sensing pathways in both inducing ERV-reactive natural antibodies and selecting ERV-reactive clones in the pre-immune repertoire.

### Example 2-8: IGHV6-3 mAb Targets Conserved Glycan Epitopes for Recognition of Exogenous Enveloped Viruses

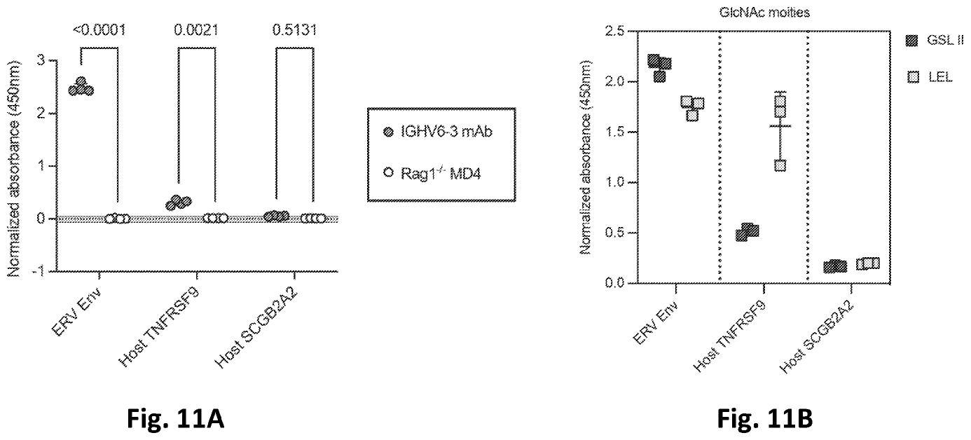

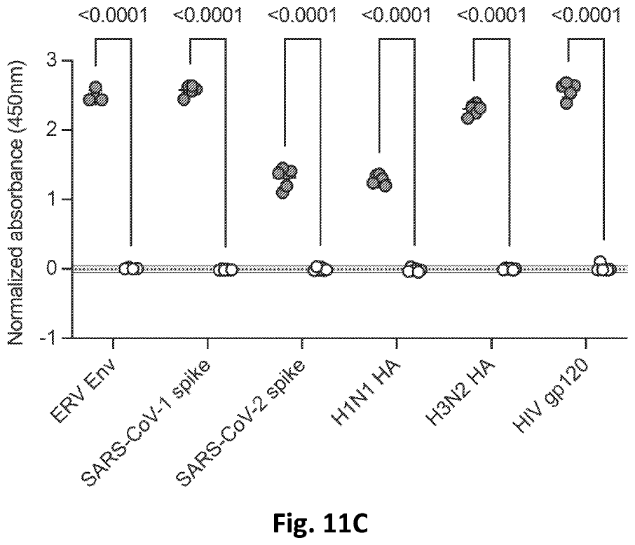

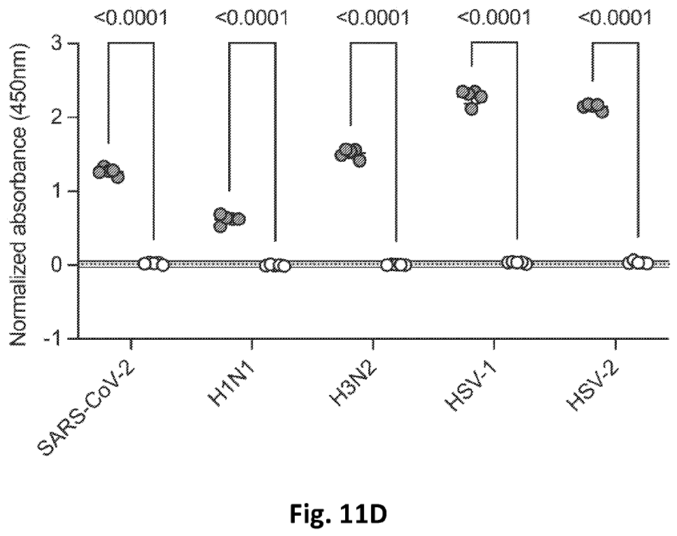

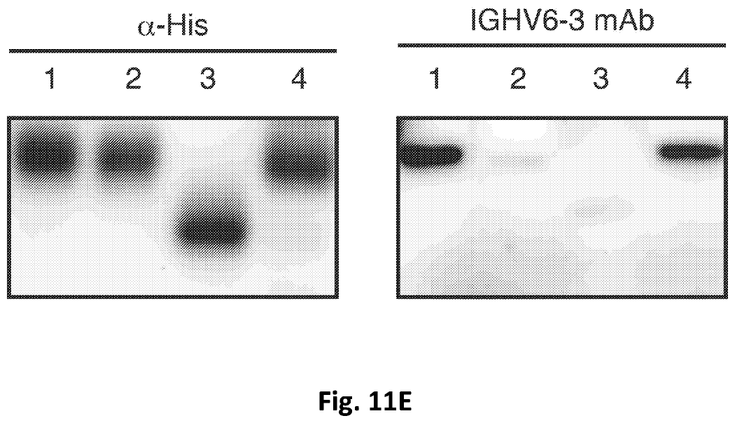

Having revealed the innate signaling-dependent regulation underlying expansion and induction of the ERV-reactive B-1 repertoire, the present study next asked whether the terminal GlcNAc epitopes recognized by ERV-reactive antibodies could serve as a “non-self” discrimination factor. It is well-established that many mammalian proteins are post-translationally modified by N-glycosylation, which is initiated by attachment of a precursor containing a GlcNAc2 mannose (Man)₃ core to an Asn residue in the endoplasmic reticulum (ER), followed by addition or removal of other monosaccharides in the ER and Golgi apparatus. Within the medial and trans Golgi, the termini of N-glycans on mammalian proteins are processed by galactosylation, sialylation and/or fucosylation, except for high mannose-type glycans. Therefore, host-derived glycoproteins also contain GlcNAc in the core structure. However, IGHV6-3 mAb did not recognize two host proteins containing predicted N-linked glycosylation sites (only by sequence-prediction) (FIG. 11A). This is also consistent with the results showing that IGHV6-3 mAb failed to recognize VLP lacking ERV surface antigens and Env lacking terminal GlcNAc (FIGS. 9B and 9D). Using Griffonia simplicifolia II lectin (GSL-II) that recognizes GlcNAcα1-4Galβ l-4GlcNAc (terminal GlcNAc), and Lycopersicon esculentum lectin (LEL) that recognizes (GlcNAcβ1-4)₁₋₄ (GlcNAc in core structures), the present study was able to detect core GlcNAc on ERV Env and TNFRSF9 while terminal GlcNAc was only detected on ERV Env (FIG. 11B).

The scope of investigation was widened to exogenous viruses considering that N-linked glycosylation of envelope proteins is a universal feature of enveloped viruses, and terminal GlcNAc may be present on other envelope glycoproteins. IGHV6-3 mAb bound to recombinant severe acute respiratory syndrome coronavirus 1 (SARS-CoV-1) spike protein, SARS-CoV-2 spike protein, influenza A/PR8 (H1N1) hemagglutinin (HA), influenza A/X-31 (H3N2) HA, and human immunodeficiency virus (HIV) gp120 (FIG. 11C). In addition to purified recombinant envelope proteins, IGHV6-3 mAb also recognized live virus particles of SARS-CoV-2, H1N1, H3N2, herpes simplex virus type 1 (HSV-1), and HSV-2 (FIG. 11D). To further investigate whether such cross-reactivity relies on conserved GlcNAc epitope, enzymatic treatment was applied to remove terminal GlcNAc, N-linked glycans, and 0-linked glycans from H1N1 HA. It was observed that terminal GlcNAc on N-linked glycans was indispensable for IGHV6-3 mAb recognition of H1N1 HA (FIG. 11E). Indeed, rapid B-1 cell secretion of IgM in response to influenza virus infection has been shown to confer early protection, contributing alongside the conventional B-2 cell-derived humoral response to combat infection. The ERV-reactive antibodies identified could, in part, contribute to B-1 mediated host defenses against influenza virus infection. Thus, relying on the recognition of a conserved glycan epitope ubiquitously displayed on envelope glycoproteins, ERV-reactive natural antibodies recognize a variety of enveloped viruses.

### Example 2-9

Endogenous retroviral proteins are transcribed and expressed by the host cells at low levels at steady state, due to incomplete silencing of ERV by epigenetic mechanisms. Furthermore, certain immunodeficiencies can render the host susceptible to ERV reactivation, underscoring the indispensable role of immune system in surveilling for ERV emergence. While previous studies identified that B cells and nucleic acid sensing by TLR7 are indispensable for ERV control, it is not known which B cell subsets and downstream effector molecules are required for this function. The present study employed fluorescence-tagged ERV particles and demonstrated that naïve peritoneal B-1 subsets express ERV-reactive BCR. In contrast to conventional B-2 BCRs that are fine-tuned by antigen-directed affinity maturation, B-1 BCRs are largely germline-restricted and considered to be evolutionarily selected for recognition of conserved epitopes present on invading pathogens or dying cells. However, B-1 BCRs are not completely invariant, as TdT expression is induced after birth, leading to non-templated nucleotide insertions seen in the B-1 cell pool replenished by adult bone marrow hematopoiesis, of which the incidence increases with age. Adding another layer to the diversity of B-1 repertoire, AID^{-/-} mediated SHM on B-1 BCR is initiated after weaning, progressively accumulates thereafter, and is enriched on class-switched isotypes. B-1 cells in this study were all isolated from 6-week-old mice. At this stage, non-templated nucleotide insertions are frequent, while the incidence of isotype-switching and SHM remains low. This is also the case for ERV-reactive BCR identified in the repertoire analysis. Whether AID^{-/-} mediated SHM will accumulate in ERV-reactive clones with age remains unexplored. Yet, it is well-established that the pre-immune repertoire is constantly being shaped by self- and environmental antigens and exhibits expansion of certain clones and decline of clonal plasticity. The timing of dramatic skewing of the pre-immune repertoire coincides with the first exposure to microbiota at birth, and recolonizing germ-free mice with normal microbiota reconstitutes IGHV6-3 clones. The self-antigen responsible for the positive selection of ERV⁻ reactive clones during fetal development in the absence of microbiota is nonetheless unclear. Moreover, similarities of the VH1 family gene usage in the B-1 repertoire developed in germ-free mice and SPF mice suggest that unknown self-antigens may help to regulate B-1 cell ontogeny. ERV envelope and glycosylated gag proteins are present on the surface of host cells and could serve as the self-antigen for ERV-reactive B-1 cell selection. Given the long co-evolution with ERV in the genome and the dynamic regulation of ERV transcripts during embryonic development, the present study proposed a model in which ERV env and/or glycogag proteins may contribute to fetal B-1 cell selection by providing positive selection cues. From an evolutionary perspective, such a model would also explain how stochastic cell-intrinsic loss of ERV repression by fetal cells could be suppressed by the immune system. Notably, memory B cells secreting only IgM, and no IgA, with cross-reactivity to both microbiota-derived antigens and ERV were identified in germ-free mice in the absence of deliberate immunization, further supporting the hypothesis that ERV, in conjunction with the microbiota, contribute to the development of pre-immune B cell repertoire.

There is a developing model that B-1 cells are heterogenous in their functionality, both maintaining the natural antibody pool and responding to infections. While B-1 cells in the spleen and bone marrow secrete natural IgM at steady-state, spontaneous secretion of IgM in peritoneal B-1 cells is inhibited by peritoneal macrophages. Although B-1 cells located in the spleen and body cavity are not completely segregated due to systemic circulation, B-1 cells in the peritoneal and plural cavities are specialized to be the rapid “responders” to infection, thereby contributing to induced natural immunity. Redistribution of such responder B-1 cells to the spleen, mesenteric lymph node, or mediastinal lymph node, where they differentiate into antibody-secreting cells, was seen in mice receiving either intraperitoneal or intranasal challenge. The present study demonstrated that anti-Env IgM was inducible upon direct TLR stimulation and by unrelated viral challenge, and the time course for induction of these antibodies preceded the initiation of the B-2 response. ERV transcription is up-regulated in the small intestine by microbial stimulation, as compared to other tissues, and depletion of commensal microbiota can prevent ERV emergence. Collectively, these data suggest that early ERV production in adult mice may originated from the gastrointestinal (GI) tract. Furthermore, exogenous MLV targets B-1 cells to establish infection, indicating that MLV takes advantage of B-1 cell specificity to its surface glycoproteins for spreading to other cell tissues. Given the cross-reactivity of natural IgM and gut IgA to commensal bacterial antigens and ERV, the selection of ERV-reactive clones by microbial antigens, and the correlation of microbial stimulation with ERV transcription, B-1 cells that reside in the peritoneal cavity may be specifically suited to the surveillance and suppression of emerging ERV.

Using the ERV-baiting strategy, the present study was able to directly profile the ERV-reactive BCR repertoire in naïve mice. Using genetic tools, the present study demonstrated the requirement for secreted antibodies to prevent ERV emergence. Further, ERV-reactive BCR sequences were identified in this study. Usage of IGHV1-53, IGHV3-6, IGHV6-3 and IGHV7-3 V_{H} genes were enriched in the ERV-reactive repertoire. Consistent with known B-1 BCR features, antibodies encoded by these V_{H} genes appeared to be broadly reactive. Although the present study did not obtain an IGHV3-6 mAb, two out of the three mAbs generated from IGHV3-6-expressing B-1 cells by other groups were reactive to microbiota. The IGHV1-53 mAb the present study generated were reactive to influenza A virus (data not shown), and murine MZB cells expressing IGHV1-53 react with HIV gp120. IGHV6-3- and IGHV7-3-expressing B-1b cells were reactive to bacterial GAC. The present study further demonstrated that the cross-reactivity of IGHV6-3 mAb stemmed from the capacity of the IGHV6-3 framework to recognize exposed terminal GlcNAc epitopes displayed on multiple pathogens, including Group A streptococcus (GAS) and enveloped viruses. Although the IGHV6-3 mAb identified is ˜87% identical to HGAC mAb developed from mice immunized with a streptococcal vaccine, it expresses a distinct CDR3 (FIG. 17B). Differences in CDR3 sequences may underlie their preference in specificities to viral-derived N-linked GlcNAc or bacterial-derived clustered O-linked GlcNAc, and further suggest shaping of the pre-immune repertoire upon antigen challenge. In contrast to bacterial products that are generated using distinct glycosylation systems in prokaryotic organisms, viruses utilize host translation and glycosylation machinery to generate their glycoproteins. It is well established that most of the viral envelope proteins are intensively glycosylated with dense glycosylation sites, which on one hand contributed to immune evasion, a so-called “glycan shield”. On the other hand, premature glycosylation could give rise to neoepitopes that permit immune recognition, of which high-mannose type glycan is a well-studied example. Future study is required to identify the biosynthetic pathways leading to the exposure of terminal GlcNAc on viral glycoproteins, but not host glycoproteins.

Notably, besides bacterial- and viral-derived terminal GlcNAc epitopes, such glycan epitopes have been identified on malignant cells and in autoimmune disease. In fact, proteins with exposed terminal GlcNAc are targeted by mannose-binding lectin (MBL) for removal in the liver, leading to short-term circulatory survival. Here the present study identified terminal GlcNAc as a conserved epitope targeted by natural antibodies to discriminate “non-self”. As natural IgM recognizes glycan modifications that did not necessarily occur at the interface between the viral entry protein and its host receptor, they are not sufficient to neutralize infecting viruses. However, as demonstrated in this study, natural antibodies do exert antiviral functions to protect the host by engaging complement. Overall, this study exemplifies the role of innate-like antiviral immunity mediated by ERV-reactive natural antibodies in protection against ERV emergence and suggests a role in the prevention of exogenous enveloped viral infections.

### Example 2-10: Materials

The materials used in the present study, as well as the sources of the materials, are listed in Table 1 below.

TABLE 1

| | | |
|---|---|---|
| REAGENT or RESOURCE | SOURCE | IDENTIFIER |

Antibodies

| | | |
|---|---|---|
| LIVE/DEAD Aqua Dead Cell | ThermoFisher | Cat# L34966 |
| Stain | | |
| 573 mouse IgM Hybridoma | Leonard Evans, Rocky | N/A |
| | Mountain Laboratories (MTA); | |
| | (Evans et al., Journal of | |
| | Virological Methods 200, 47- | |
| | 532014) | |
| 83A25 rat IgG2A Hybridoma | Leonard Evans, Rocky | N/A |
| | Mountain Laboratories (MTA); | |
| | (Evans et al., J Virol 64, 6176- | |
| | 6183,1990) | |
| mAb 34 mouse IgG2b | Gifted from W. Mothes, Yale | N/A |
| | University; (Chesebro et al., | |
| | Virology 127, 134-148, 1983) | |
| Rat IgG2a isotype | BD Pharmingen | Cat# 553992; RRID: |
| | | AB_395189 |
| Biotin mouse anti-rat IgG2a | BD Pharmingen | Cat# 553894; RRID: |
| | | AB_395122 |
| PE Streptavidin | BioLegend | Cat# 405245 |
| Alexa Fluor ™ 647-R- | Invitrogen | Cat# S20992 |
| Phycoerythrin Streptavidin | | |
| Goat anti-Mouse IgG (H + L), | Invitrogen | Cat# A28181, RRID: |
| APC | | AB_2536165 |
| F(ab′)₂ Goat Anti-Mouse IgM, | Jackson | Cat# 115-136-075; RRID: |
| APC | | AB_2338650 |
| Goat Anti-Mouse IgG, Human | Southern Biotech | Cat# 103005; RRID: |
| ads-HRP | | AB_2619742 |
| Goat Anti-Mouse IgM, Human | Southern Biotech | Cat# 1020-05; RRID: |
| ads-HRP | | AB_2794201 |
| Goat Anti-Mouse Ig, Human ads- | Southern Biotech | Cat# 1010-05; RRID: |
| HRP | | AB_2728714 |
| Goat Anti-Mouse Ig, Human ads- | Southern Biotech | Cat# 1010-01; RRID: |
| UNLB | | AB_2794121 |
| Goat anti-Rat IgG (H + L) | ThermoFisher | Cat# 31470; RRID: |
| Secondary Antibody, HRP | | AB_228356 |
| His-Tag (D3I1O) XP ® Rabbit | Cell signaling | Cat# 12688; RRID: |
| mAb, HRP | | AB_2797993 |
| Mouse IGHV1-53 mAb, g2c | This study | N/A |
| Mouse IGHV6-3 mAb, g2c | This study | N/A |
| Mouse IGHV7-3 mAb, g2c | This study | N/A |
| Mouse DS6-7_C1 mAb, g2c | This study | N/A |
| Mouse DS6-7_C2 mAb, g2c | This study | N/A |
| Mouse DS6-7_C3 mAb, g2c | This study | N/A |
| Mouse DS7-6_C1 mAb, g2c | This study | N/A |
| Mouse DS7-6_C2 mAb, g2c | This study | N/A |
| Mouse DS7-6_C3 mAb, g2c | This study | N/A |
| Mouse DS1-6_C1 mAb, g2c | This study | N/A |
| Mouse DS1-6_C2 mAb, g2c | This study | N/A |
| Mouse DS1-6_C3 mAb, g2c | This study | N/A |
| Mouse monoclonal anti- | Gifted by J. Kearney, | N/A |
| Lancefield Group A Carbohydrate | University of Alabama at | |
| (clone HGAC78, m) | Birmingham; (Nahm et al., The | |
| | Journal of Immunology 129, | |
| | 1513, 1982) | |
| Mouse monoclonal anti- | Gifted by J. Kearney, | N/A |
| Lancefield Group A Carbohydrate | University of Alabama at | |
| (clone HGAC39, g3) | Birmingham; (Greenspan and | |
| | Davie, J Immunol 134, 1065- | |
| | 1072, 1985) | |
| Anti-mouse CD45, Alexa Fluor | BioLegend | Cat# 103122; RRID: |
| 488 | | AB_493531 |
| Anti-mouse CD3, Brilliant Violet | BioLegend | Cat# 100237; RRID: |
| 605TM | | AB_2562039 |
| Anti-mouse CD19, Brilliant | BioLegend | Cat# 115538; RRID: |
| Violet 421 ™ | | AB_11203527 |
| Anti-mouse CD5, FITC | BioLegend | Cat# 100606; RRID: |
| | | AB_312735 |
| Anti-mouse CD23, Brilliant | BioLegend | Cat# 101623; RRID: |
| Violet 510 ™ | | AB_2563705 |
| Anti-mouse CD21/35, | BioLegend | Cat# 123417; RRID: |
| APC/Cyanine7 | | AB_1953274 |

Bacterial and Virus Strains

| | | |
|---|---|---|
| One Shot ™ TOP10 | ThermoFisher | Cat# C404010 |
| ERV | (Treger et al., 2019b) | N/A |
| SARS-CoV-2 | BEI Resources | NR-52281 |
| A/Puerto Rico/8/34 (H1N1) | Gifted by H. Hasegawa, | N/A |
| | National Institute of Infectious | |
| | Diseases, Tokyo | |
| A/Udorn/307/72 (H3N2) | Gifted by R. Lamb, | N/A |
| | Northwestern University | |
| HSV-1 | Gifted by D. Knipe, Harvard | N/A |
| | Medical School | |
| HSV-2 | Gifted by D. Knipe, Harvard | N/A |
| | Medical School | |

Chemicals, Peptides, and Recombinant Proteins

| | | |
|---|---|---|
| EZ-Link ™ Sulfo-NHS-Biotin | ThermoFisher | Cat# 21326 |
| Standard LPS | InvivoGen | Cat# tlrl-eblps |
| R848 (Resiquimod) | InvivoGen | Cat# tlrl-r848 |
| Pam3CSK | InvivoGen | Cat# tlrl-pms |
| CpG | TriLink BioTechnologies; | N/A |
| | (Shen and Iwasaki, 2006) | |
| Poly(I:C) | InvivoGen | Cat# tlrl-picw |
| SLR14 | (Linehan Melissa et al. Science | N/A |
| | Advances 4, e1701854) | |
| Halt Protease Inhibitors | ThermoFisher | Cat# 78446 |
| ThermoScientific | | |
| TRIzol Reagent | Invitrogen | Cat# 15596018 |
| ERV Envelope protein, His Tag | This study | N/A |
| ERV Glycogag protein, His Tag | This study | N/A |
| SARS-CoV-2 (2019-nCoV) Spike | Sinobiological | Cat# 40591-V08H |
| S1-His Recombinant Protein | | |
| HIV-1 gp120 Protein (group M, | Sinobiological | Cat# 11233-V08H |
| subtype CRF07_BC) (His Tag) | | |
| Influenza A H1N1 (A/Puerto | Sinobiological | Cat# 11684-V08H |
| Rico/8/1934) Hemagglutinin/HA | | |
| Protein (ECD,His Tag) | | |
| Influenza A H3N2 (A/X-31) | Sinobiological | Cat# 40059-V08H |
| Hemagglutinin/HA Protein (His | | |
| Tag) | | |
| SARS S1 protein, His Tag | Arco Biosystems | Cat# SIN-S52H5 |
| TNFRSF9, His Tag | Gifted by A. Ring, Yale | N/A |
| | University | |
| SCGB2A2, His Tag | Gifted by A. Ring, Yale | N/A |
| | University | |
| IL-1R, His Tag | Gifted by A. Ring, Yale | N/A |
| | University | |
| N-acetylglucosamine | Vector Lab | Cat# S-9002 |
| α-methylmannoside | Vector Lab | Cat# S-9005 |

Critical Commercial Assays

| | | |
|---|---|---|
| Ni-NTA Agarose | QIAGEN | Cat# 30210 |
| Pierce ™ Protein G Agarose | ThermoFisher | Cat# 20397 |
| iTaq ™ Universal SYBR ® Green | Bio-Rad | Cat# 1725124 |
| Supermix | | |
| SuperSignal ™ West Pico PLUS | ThermoFisher | Cat# 34577 |
| Chemiluminescent Substrate | | |
| Pierce ™ ECL Western Blotting | ThermoFisher | Cat# 32209 |
| Substrate | | |
| eBioscience ™ TMB Solution | Invitrogen | Cat# 00-4201-56 |
| (1X) | | |
| Shrimp Alkaline Phosphatase | ThermoFisher | Cat# M0371S |
| (rSAP) | | |
| T4 DNA Ligase | NEB | Cat# M0202S |
| iScript ™ cDNA Synthesis Kit | Bio-Rad | Cat# 1708890 |
| SuperScript ™ III CellsDirect ™ | ThermoFisher | Cat# 18080200 |
| CDNA Synthesis Kit | | |
| Retro-X ™ Concentrator | TAKARA | Cat# 631456 |
| Lipofectamine ™ 2000 | ThermoFisher | Cat# 11668019 |
| Transfection Reagent | | |
| Gibson Assembly ® Master Mix | NEB | Cat# E2611L |
| Biorad Precision plus protein | Biorad | Cat# 1610374 |
| ladder | | |
| Easy Sep ™ Mouse CD4 T cell | STEMCELL | Cat# 19852 |
| isolation kit | | |
| Easy Sep ™ Mouse Pan-B Cell | STEMCELL | Cat# 19844 |
| Isolation Kit | | |
| Expi293 ™ Expression Medium | ThermoFisher | Cat# A1435101 |
| ExpiFectamine ™ 293 | ThermoFisher | Cat# A14524 |
| Transfection Kit | | |
| Lipofectamine ™ 2000 | Invitrogen | Cat# 11668019 |
| Transfection reagent | | |
| O-Glycosidase & Neuraminidase | NEB | Cat# E0540S |
| Bundle | | |
| PNGase F | NEB | Cat# P0709S |
| β-N-Acetylglucosaminidase S | NEB | Cat# P0744S |
| Q5 ® High-Fidelity 2X Master | NEB | Cat# M0492S |
| Mix | | |
| Lectin Kit III, Biotinylated | Vector Lab | Cat# BK-3000 |
| VECTASTAIN ® Elite ABC-HRP | Vector Lab | Cat# PK-6100 |
| Kit, Peroxidase (Standard) | | |
| Carbo-Free Blocking Solution | Vector Lab | Cat# SP-5040-125 |
| (10x Concentrate) | | |
| In-Fusion ® Snap Assembly | TAKARA | Cat# 638948 |
| Master Mix | | |
| Gibson Assembly ® Master Mix | NEB | Cat# E2611L |
| Zymoclean Gel DNA Recovery | ZYMO RESEARCH | Cat# D4008 |
| Kit | | |
| DNA Clean & Concentrator ™ | ZYMO RESEARCH | Cat# D4005 |
| Plasmid Plus Midi Kit | QIAGEN | Cat# 12945 |
| QIAprep Spin Miniprep Kit | QIAGEN | Cat# 27104 |
| RNeasy Mini Kit | QIAGEN | Cat# 74104 |
| QIAShredder | QIAGEN | Cat# 79654 |
| Plasmid Plus Maxi Kit | QIAGEN | Cat# 12963 |
| Amicon Ultra-15 Centrifugal | Millipore | Cat# UFC901024 |
| Filter Unit, 10,000 MWCO | | |
| Vivaspin ® 2 30,000 MWCO | GE Healthcare | Cat# VS0291 |
| Poly-Prep ® Chromatography | Bio-Rad | Cat# 7311550 |
| Columns | | |
| Econo-Pac ® Chromatography | Bio-Rad | Cat# 7321010 |
| Columns | | |
| Deposited Data | | |
| ERV-B-1 Single-cell V(D)J | | |
| sequencing data | | |
| ERV-B-1 Single-cell 5′ gene | | |
| sequencing data | | |

Experimental Models: Cell Lines

| | | |
|---|---|---|
| 293T | ATCC | CRL-3216; RRID: |
| | | CVCL_0063 |
| Expi293F ™ | Gibco | Cat# A14527 |
| DFJ8 avian fibroblast | Gifted by W. Mothes, Yale | N/A |
| | University | |

Experimental Models: Organisms/Strains

| | | |
|---|---|---|
| Mice:C57BL/6N | Charles River | Cat# C57BL/6NCrl |
| Mice: B6.129S7-Rag 1^{tm1Mom}/J | Jackson | Cat# 002216; RRID: |
| (Rag1^{-/-}) | | IMSR_JAX:002216 |
| Mice: C57BL/6- | Jackson | Cat# 002595; RRID: |
| Tg(IghelMD4)4Ccg/J | | IMSR_JAX:002595 |
| Mice: Rag1^{-/-}MD4 Tg | This study | N/A |
| Mice: Tlr7^{-/-} (C57BL/6N) | (Lund et al., Proceedings of the | N/A |
| | National Academy of Sciences | |
| | 101, 5598-5603, 2004) | |
| Mice: mS^{-/-}(sIgM^{-/-}) | Gifted by N. Baumgarth, | N/A |
| | University of California, Davis | |
| Mice: AID^{-/-} | Gifted by D. Schatz, Yale | N/A |
| | University | |
| Mice: C3^{-/-} | Gifted by J. Hendrickson, Yale | N/A |
| | University | |
| Mice: AID^{-/-}sIgM^{-/-} | This study | N/A |
| Mice: AID^{-/-}C3^{-/-} | This study | N/A |
| Mice: Germ-free C57BL/6 | Gifted by N. Palm, Yale | N/A |
| | University | |

Oligonucleotides

| | | |
|---|---|---|
| Primer: Spliced Emv2 Env | IDT (Young et al., Nature 491, | N/A |
| Forward: 5′- | 774-778, 2012) | |
| CCAGGGACCACCGACCCACC | | |
| GT-3′ (SEQ ID NO: 11) | | |
| Primer: Spliced Emv2 Env | IDT (Young et al., Nature 491, | N/A |
| Reverse: 5′- | 774-778, 2012) | |
| TAGTCGGTCCCGGTAGGCCT | | |
| CG-3′ (SEQ ID NO: 12) | | |
| Primer: GAPDH Forward: 5′- | IDT | N/A |
| GAAGGTCGGTGTGAACGGA- | | |
| 3′ (SEQ ID NO: 13) | | |
| Primer: GAPDH Reverse: 5′- | IDT | N/A |
| GTTAGTGGGGTCTCGCTCCT- | | |
| 3′ (SEQ ID NO: 14) | | |
| MLV_Pol Forward: 5′- | IDT (Lötscher et al., PLOS One | N/A |
| TGGCTGACTGAGGCTAGAAA | 2, e1158, 2007) | |
| AGAGACTGT-3′ (SEQ ID NO: | | |
| 15) | | |
| MLV Pol Reverse: 5′- | IDT (Lötscher et al., PLOS One | N/A |
| GCTTTTTGGACAGGTAGGCC | 2, 2007) | |
| AC-3′ (SEQ ID NO: 16) | | |
| Primer: FMLV-IF-1F: 5′- | IDT | N/A |
| GGCCTCCCTCCTGACCTTAG | | |
| ACG-3′ (SEQ ID NO: 17) | | |
| Primer: FMLV-IF-1R: 5′- | IDT | N/A |
| TAAGGGGATTAGGAGGTCCC | | |
| GCG-3′ (SEQ ID NO: 18) | | |
| Primer: FMLV-IF-2FS: 5′- | IDT | N/A |
| GGCTTTAGTCCTGACTCAAC | | |
| AATACC-3′ (SEQ ID NO: 19) | | |
| Primer: FMLV-IF-2FM: 5′- | IDT | N/A |
| CGCGGGACCTCCTAATCCCC | | |
| TTACCAGCTAAAACCACTAG | | |
| AATACGAGCC-3′ (SEQ ID NO: | | |
| 20) | | |
| Primer: FMLV-IF-2R: 5′- | IDT | N/A |
| TTTCCATAGGCTCCGCCCCC- | | |
| 3′ (SEQ ID NO: 21) | | |
| Primer: FMLV-IF-3F: 5′- | IDT | N/A |
| GGGGCGGAGCCTATGGAAA | | |
| AACG-3′ (SEQ ID NO: 22) | | |
| Primer: FMLV-IF-3R: 5′- | IDT | N/A |
| CGTCTAAGGTCAGGAGGGA | | |
| GGCC-3′ (SEQ ID NO: 23) | | |

Recombinant DNA

| | | |
|---|---|---|
| Plasmid: pRVL-2 | Addgene | Cat# 104580 |
| Plasmid: pRVL-1 | Addgene | Cat# 104579 |
| Plasmid: FMLV-DEnvDGlycogag | This study | N/A |
| Plasmid: | This study | N/A |
| pEZT_MLV_Env_SU_M | | |
| Plasmid: | This study | N/A |
| pEZT_MLV_Env_SU_V | | |
| Plasmid: pUC19-ERV | (Treger et al., J Virol 93, | N/A |
| | 2019b) | |
| Plasmid: pRVL-2_IGHV1-53 | This study | N/A |
| Plasmid: pRVL-2_IGHV6-3 | This study | N/A |
| Plasmid: pRVL-2_IGHV7-3 | This study | N/A |
| Plasmid: pRVL-1_IGKV4 | This study | N/A |
| Plasmid: pRVL-1_IGKV14 | This study | N/A |
| Plasmid: pRVL-1_IGLV1 | This study | N/A |
| Plasmid: pRVL-2_DS6-7_C1 | This study | N/A |
| Plasmid: pRVL-2_DS6-7_C2 | This study | N/A |
| Plasmid: pRVL-2_DS6-7_C3 | This study | N/A |
| Plasmid: pRVL-2_DS7-6_C1 | This study | N/A |
| Plasmid: pRVL-2_DS7-6_C2 | This study | N/A |
| Plasmid: pRVL-2_DS7-6 C3 | This study | N/A |
| Plasmid: pRVL-2_DS1-6_C1 | This study | N/A |
| Plasmid: pRVL-2_DS1-6 C2 | This study | N/A |
| Plasmid: pRVL-2_DS1-6 C3 | This study | N/A |

Software and Algorithms

| | | |
|---|---|---|
| FlowJo | TreeStar Inc | www.flowjo.com/ |
| GraphPad Prism 9 | GraphPad Software | www.graphpad.com/ |
| Cell Ranger | 10X Genomics | support.10xgenomics.com/ |
| | | single-cell-gene- |
| | | expression/software/ |
| | | overview/welcome |
| Change-O | (Gupta et al., Bioinformatics | changeo.readthedocs.io/ |
| | 31, 3356-3358, 2015) | en/stable/ |
| Alakazam | (Gupta et al., Bioinformatics | alakazam.readthedocs.io/ |
| | 31, 3356-3358, 2015) | en/stable/ |
| SHazaM | (Gupta et al., Bioinformatics | shazam.readthedocs.io/ |
| | 31, 3356-3358, 2015) | en/stable/ |
| Treemap | Martijn Tennekes | CRAN.R- |
| | | project.org/package- |
| | | treemap |
| Seurat V3 | (Stuart et al., Cell 177, 1888- | satijalab.org/seurat/ |
| | 1902.e1821, 2019) | index.html |
| ScRepertoire | (Borcherding et al., Nature 294, | ncborcherding.github.io/ |
| | 88-90, 2020) | vignettes/vignette.html |
| Enrichr | (Xie et al., Curr Protoc 1, e90, | cran.r- |
| | 2021) | project.org/web/packages/ |
| | | enrichR/index.html |
| TMHMM - 2.0 | DTU Health Tech | services.healthtech.dtu. |
| | | dk/service.php?TMHMM-2.0 |
| IgBLAST | (Ye et al., Nucleic Acids | bio.tools/igblast |
| | Research 41, W34-W40. 2013) | |
| msa | (Bodenhofer et al., | bioconductor.org/packages/ |
| | Bioinformatics 31, 3997-3999, | release/bioc/html/msa.html |
| | 2015) | |
| IMGT/High-Vquest | (Lefranc et al., Nucleic Acids | www.imgt.org |
| | Research 43, D413-D422, | |
| | 2015) | |

### Example 2-11: Methods

DFJ8 coculture assay

Co-culture assay were performed as previously described (Jayewickreme et al., Frontiers in Immunology 12, 2022). DFJ8 cells were pre-seeded at 100,000 cells/mL in 12-well-plate one day prior assay with 1 mL Dulbecco's Modified Eagle Medium (DMEM) per well. Splenocytes were isolated according to the cell isolation method described in this study and added into DFJ8 cell culture with 1.5 mL Roswell Park Memorial Institute (RPMI) medium plus 0.5 mL DMEM. Four days after co-culturing, cell and supernatant were altogether transferred to 60 mm tissue culture (TC) dish and cultured for additional three days, then were transferred to 10 cm TC dish, and cultured for seven days. On Day 14 post initial co-cultured in 12-well-plate, cells were harvested and stained for mouse CD45 (BioLegend) and for ERV Env by mAb 573 (kindly provided by Leonard Evans, NIH) (Evans et al., Journal of Virological Methods 200, 47-53. 2014). Percentage of ERV infected cells were quantified in CD45-DFJ8 cells.

Reverse transcription-quantitative polymerase chain reaction (RT-qPCR)

RNA was isolated from peripheral blood using the RNeasy mini kit (QIAGEN) according to the manufacturer's manual with the DNA digestion step included. Reverse transcription of isolated RNA was performed using the iScript™ cDNA Synthesis Kit (Bio-rad) in 40 μL reactions. Quantitative PCR was performed using iTaq™ Universal SYBR® Green Supermix (Bio-rad) in 10 μL reactions in triplicate. Each PCR reaction contained 30 ng cDNA and primers were used at a final concentration of 0.225 μM. Primer sets used in this study are listed in the key resources table.

Enzyme-linked immunosorbent assays

96-well EIA/RIA plates (Corning) were coated at 4° C. overnight with 1 μg/mL purified recombinant proteins or 10⁶ pfu/mL virions in carbonate buffer at a final volume of 100 μL per well in duplicate. Wells coated with carbonate buffer only (empty wells) were used to measure background signal caused by non-specific binding. Plates were blocked with 5% fetal bovine serum (FBS) in phosphate buffer saline (PBS) for 1 hour at room temperature (RT). In all ELISA conducted with recombinant mAb and HGAC mAb, plates were blocked with Carbo-Free Blocking solution (Vector Lab) to reduce background binding to FBS-derived glycans. Primary incubations were conducted at 4° C. overnight with diluted sera ( 1/50 dilution), cell culture supernatant (undiluted), or mAb (3 μg/mL) in 100 μL blocking solution per well. Following primary adsorption, bound antibodies was detected by alkaline-phosphatase (HRP)-conjugated mouse Ig isotype-specific secondary antibodies (Southern Biotechnology) at a final concentration of 1 μg/mL in 100 μL blocking solution at RT for 1 hour. TMB substrate solution (Invitrogen) was added into plates to initiate the phosphatase reaction, which was then terminated by addition of stop solution (2N H₂SO₄). Plate absorbances were subsequently measured by 450 nm absorbance (with 490 nm absorbance subtracted). Background signal of empty wells following primary and secondary incubation were further subtracted to normalize for background binding in each sample. For detection of total antibody input signal, the plates were coated with 0.1 μg of unlabeled anti-mouse Ig antibody (Southern Biotechnology) in 100 μL carbonate buffer followed by primary and secondary incubation. In the competitive ELISA, mAb solution (3 μg/mL) was preabsorbed at the indicated concentration of monosaccharide (0 mM-200 mM) at RT for 2 hours, and then was applied to antigen-coated plate, followed by secondary antibody incubation.

For measurement of GlcNAc by lectins, plates were coated with purified recombinant protein at a final concentration of 3 μg/mL in carbonate buffer (100 μL/well) at 4° C. overnight. Plates were blocked with Carbo-Free blocking solution. Biotinylated lectins (Vector Lab) at a final concentration of 5 μg/mL in blocking buffer were applied to wells and the plates were incubated at RT for 30 min. For each lectin, the negative control was made by pre-adsorption of lectins with 200 mM of free GlcNAc at RT for 2 hours. Lectins without blocking sugars were also incubated at RT for 2 hours to normalize for lectin activity. Bound lectins were detected using VECTASTAIN® Elite ABC-HRP Kit (Vector Lab) according to the manufacturer's manuals. TMB substrate solution and stop solution were subsequently added to carry out the peroxidase reaction. Plates were read at 450 nm and 490 nm. For each lectin, the absorbance of respective negative controls was subtracted.

Recombinant protein production

Env Glycogag sequences were cloned from Emv2 viral sequence (Treger et al., J Virol 93, 2019) using Q5© High-Fidelity 2×Master Mix (NEB) with overlapping arms for inserting into linearized pEZT expression vectors (kindly gifted by Aaron Ring, Yale University) using Gibson Assembly® Master Mix (NEB). Env SU(M) encodes for a protein starting from the Methionine (1) of Env, generating proteins containing the predicted transmembrane N-terminal domain. Env SU(V) encodes for a protein starting from the Valine (50) of Env, generating proteins lacking the N-terminal transmembrane helix. Predicted transmembrane helices at the C-terminal were not included in either Env SU construct. After sequences were confirmed by sanger sequencing, expression plasmids were purified using Plasmid Plus Maxi Kit (QIAGEN). 100 mL of Expi293F™ (Gibco) culture were transduced with 100 μg of Env SU or Glycogag expression vectors using ExpiFectamine™ 293 Transfection Kit (ThermoFisher) according to the manufacturer's manuals. Cell culture supernatants were harvested on day 4 post-transduction and were adjusted to the salt concentration of 300 nM NaCl and 20 mM Tris-HCl (pH 8) at a final volume of twice the original volume of harvested supernatant. 1 mL of Ni-NTA Agarose beads (QIAGEN) were incubated with supernatants while stirring at 4° C. for 3 hours. The mixtures were loaded onto gravity-flow Econo-Pac© Chromatography Columns (Bio-Rad). Packed beads were washed with 5× column volumes (CV) of PBS and 5× CV of 20 mM imidazole in PBS. Bound proteins were eluted with 250 mM imidazole in PBS. Absorbance of the eluates at 280 nm were monitored to estimate the amount of residual proteins and to determine elution volumes. The elutes were concentrated by centrifugation using 30,000 MWCO filter units (GE Healthcare). Buffer exchange was performed in the same filter unit by adding 3× elution volume of PBS. The concentration of purified proteins was determined from the absorbance at 280 nm. Purified proteins were stored at −80° C. for further analysis.

ERV and VLP generation

Single cell colonies of DFJ8 cells co-cultured with Tlr7—splenocytes that stably express high levels of ERV were cultured for 7 days to amplify ERV particles as described previously (Treger et al., J Virol 93 2019b). Culture supernatants were centrifuged and passed through a 0.45 μm filter to remove DFJ8 cells and cell debris and then concentrated by ultracentrifugation for 23,000×g over 25% sucrose. The pelleted ERV were resuspended in optiMEM media and stored at −80° C. for future analysis. The FMLV-ΔGlycogag vector was kindly gifted by Walther Mothes, Yale University. To generate FMLV-ΔEnvΔGlycogag vector, seamless DNA cloning was performed using In-Fusion Snap Assembly Master Mixes (TAKARA) according to the manufacturer's manual to delete the Env coding sequence. Fragment 1 was amplified by the FMLV-IF-1F and FMLV-IF-1R primer pair. Fragment 2 was first amplified by the FMLV-IF-2FS and FMLV-IF-2R primer pair, and then amplified with FMLV-IF-2FM and FMLV-IF-2R primers to avoid multiple primer binding sites. Fragment 3 was amplified by the FMLV-IF-3F and FMLV-IF-3R primer pair. All primer sequences are listed in the key resources table and all PCR reactions were conducted using Q5© High-Fidelity 2× Master Mix (NEB). After seamless cloning of all amplified fragments, the joined plasmid was recovered from transformed One Shot™ TOP10 E. coli (ThermoFisher) and Sanger sequencing was performed to confirm the sequence across the entire plasmid. 293T cells were transfected with FMLV-ΔEnvΔGlycogag and VSV-G vectors using Lipofectamine™ 2000 (Invitrogen) according to the manufacturer's manual. 24 hours after transduction, the media was replaced. 48 hours after transduction, supernatants containing pseudotyped viruses were harvested and virus was concentrated using Retro-X™ Concentrator (TAKARA). DFJ8 cells seeded the day before transduction were infected with concentrated virus in OptiMEM containing 5 μg/mL polybrene. After 2.5 hours, the infected media was removed and replaced with fresh DMEM. Seven days after transduction, DFJ8 cells were harvested and stained for envelope protein expression using mAb 573 (Evans et al., Journal of Virological Methods 200, 47-53, 2014) and for Glycogag protein expression using mAb 34 (Chesebro et al., Virology 127, 134-148, 1983 and Robertson et al., J Virol 82, 408-418, 2008). Goat anti-mouse IgM APC and Goat anti-mouse IgG (H+L) APC were used to detect bound mAb 573 and mAb 34, respectively. DFJ8 cells transduced with FMLV-ΔEnv and FMLV-ΔGlycogag were used as controls for staining. After confirming knockout of Env and Glycogag, transduced DFJ8 cells were cultured for 7 days to amplify VLP, which were subsequently purified by ultracentrifugation as described above.

ERV and VLP quantification

10 μL of concentrated ERV or VLP were lysed in 990 μL of TRIzol reagent (Invitrogen) and homogenized by vigorous vortexing. 200 μL of chloroform were added to each sample, followed by vortexing. The aqueous layer was isolated by centrifugation at 12,000×g for 15 min at 4° C., and 500 μL of isopropanol and 5 μg of glycogen were added to the aqueous phase. RNA was then pelleted by centrifugation at 12,000×g for 10 min at 4° C. and washed twice with ice-cold 75% ethanol. After removing all liquid, the RNA pellets were dried in a tissue culture hood and resuspended in 10 μL of RNase-free water. Viral cDNAs were synthesized using the SuperScript III Cells Directed cDNA synthesis Kit (Invitrogen) with all isolated RNA, and qPCR was performed as described above using 1 μL of the cDNA reaction (undiluted, 1/10 diluted and 1/100 diluted) and the MLV_Pol primer set (sequence listed in the key resources table). A standard Curve for viral RNA copy numbers was generated by qPCR of the pUC19-ERV plasmid encoding full length ERV sequence (Treger et al., J Virol 93, 2019), and viral RNA copy number and concentration were quantified according to this standard curve.

Virions biotinylation and streptavidin conjugation

The following protocol was developed for preparation of 400 μL of 108 tagged ERV or VLP (4.14×10⁻¹⁰ mM), which were freshly prepared prior to each ERV-baiting assay. The calculated volume of virions was diluted to a final volume of 1 mL with sterile PBS. The diluted virions were added to a 15 mL 10,000 MWCO centrifugal filter unit (Millipore) containing 11 mL of PBS and centrifuged at 5000×g for 50 min at RT to remove OptiMEM from solution. Residual solution in the unit containing concentrated virions was transferred to a new Eppendorf tube. The filter unit was washed with 200 μL of PBS, which was added to the same Eppendorf tube. EZ-link™ Sulfo-NHS Biotin (ThermoFisher) was reconstituted with RNase-free water and 16.5 μL of 5 mM biotin was added to PBS containing virions to achieve 5×10⁵ molar excess of biotin for each mole of virions. Additional PBS was added to bring the total volume to 400 μL and the reaction was incubated at RT for 1 hour. After biotinylation, the 400 μL reaction was transferred to a new 10,000 MWCO centrifugal filter unit containing 11.6 mL of 100 mM glycine in PBS and incubated for 30 min at RT to quench unconjugated biotin. Samples were centrifuged at 5000×g for 30 min and washed twice with 12 mL of PBS to remove excess unconjugated biotin and glycine. The residual solution containing biotinylated virions was transferred to a new Eppendorf tube, and the filter unit was washed with PBS as above to capture any remaining virions in the filter. The final volume of biotinylated virions was adjusted to 365 μL using PBS, and 35 μL of 1 mg/mL PE-SA (BioLegend) or PE-SA-AF647 (Invitrogen) were added to the ERV⁻ and VLP-containing solution, respectively. The reaction was incubated covered at RT for 1 hour. The tagged virions in solution were then transferred to a new 2 mL Eppendorf tube and 10% FBS in PBS was added to bring total volume to 1.5 mL. The sample was underlaid with 200 μL of 15% sucrose in PBS and centrifuged at 12,000× rpm at 4° C. for 2 hours to remove excess SA-fluorophore. The pelleted virions were resuspended in 400 μL of 10% FBS in PBS.

Cell isolation and culture

To isolate PBMC, mice were anesthetized with isoflurane and blood was collected by retro-orbital bleed using heparinized tubes (Fisher Scientific). Collected blood was directly resuspended in 8 mM EDTA in PBS. Red blood cells were lysed with ACK lysis buffer (150 mM NH₄Cl, 1 M KHCO₃, 0.1 mM EDTA, pH 7.4) and nucleated cells were pelleted by centrifugation and then washed twice with PBS. To isolate RNA, 350 μL of RLT buffer (QIAGEN) containing β-mercaptoethanol were added for cells isolated from 100 μL of whole blood, lysed by a QIAshredder (QIAGEN) column, and stored at −80° C. for further analysis.

To isolate splenocytes, the spleens were isolated from euthanized mice and dissociated through 70 μm filters in 5% FBS in RPMI media. Red blood cells were lysed by ACK lysis buffer and cells were pelleted by centrifugation. Cell debris and connective tissue present in the resuspended splenocytes were removed using a 40 μM filter, and splenocytes were washed with 5% FBS in PBS. Isolation of CD4⁺T cells or bulk B cells was performed using EasySep™ Mouse CD4 T cell isolation kit (STEMCELL) or EasySep™ Mouse Pan-B Cell Isolation Kit (STEMCELL) according to the manufacturer's manuals. Cells were resuspended in 10% FBS in PBS for subsequent use in the ERV-baiting assay, or in RPMI media for subsequent in vitro culture.

To isolate peritoneal cells, peritoneal wash was obtained by injecting 3-6 mL of 2% FBS with 2 mM EDTA in PBS into the peritoneal cavity. Following gentle shaking, the whole fluid was withdrawn by syringe and placed into 15 mL conical tubes. Peritoneal cells were then pelleted by centrifugation and washed with PBS. Isolation of bulk B cells was performed using the EasySep™ Mouse Pan-B Cell Isolation Kit (STEMCELL) according to the manufacturer's manuals. Cells were resuspended in 10% FBS in PBS for use in the ERV⁻ baiting assay or in RPMI media for in vitro culture.

To culture isolated B cells, 1,000,000 splenic B cells or peritoneal B cells were plated in 1 mL RPMI media in a 12-well-plate. For FACS-sorted B cells, 10,000 cells were plated in 200 μL RPMI media in a 96-well-plate. Where applicable, ligand for innate sensors were added to a final concentration as followed: Pam3CSK 0.5 μg/mL; LPS 5 μg/mL; R848 1 μg/mL; CpG 2.5 μg/mL; Poly(I:C) 10 μg/mL; SLR14 10 μg/mL (for SLR14, 2 μL/well of lipofectamine were added in addition to the ligands) (sources are listed in the key resources table). After five days of incubation, supernatants were harvested and anti-Env antibodies were measured by ELISA, as described in this study.

ERV-baiting assay

For each sample, 500,000 splenocytes or peritoneal cells were plated in 100 μL of 10% FBS in PBS in a round bottom 96-well-plate. Cells were incubated with 25 μL of SA-PE-AF647 tagged VLP for 30 min at RT while covered by foil, and then equilibrated to 4° C. before addition of ERV. Next, 25 μL of ice-cold SA-PE tagged ERV were added to each sample and the total volume was adjusted to 200 μL with 10% FBS in PBS. The plate was then incubated covered at 4° C. for 30 min. After incubation, all samples were washed three times with 200 μL of ice-cold PBS before staining with anti-mouse CD3-BV605, CD19-BV421, CD5-FITC, CD23-BV510, CD21/35-APC/Cy7 (BioLegend, listed in the key resources table) for 15 min at 4° C. while covered by foil. Two washes with 200 μL PBS were performed between all steps. Flow cytometry data were analyzed with FlowJo. For further analysis, targeted population of cells were sorted into RPMI media by performing FACS on the FACSAria cell sorter at Yale Flow Cytometry Facility. Sorted cells were immediately processed for 10× sequencing or for in vitro culture as described in this study.

10× Single-cell sequencing and analysis

Sorted ERV-reactive B-1 cells and total naïve B-1 cells were counted and loaded into the Chromium Controller (10× Genomics) for single-cell partitioning and barcoding. Single-cell V(D)J libraries were generated using the Chromium Single Cell V(D)J Reagent kit (10× Genomics) while single-cell gene expression libraries were generated using the Chromium Single Cell 5′ Reagent kit (10× Genomics), per the manufacturer's instruction. Libraries were sequenced on the NovaSeq 6000 Sequencing System (Illumina). Single-cell V(D)J libraries were sequenced with 2 ×150 bp paired-end reads and single-cell gene expression libraries were sequenced with 2 ×100 bp paired-end reads. Details for the sequencing matrix are listed in the supplemental information (Table S1 and S2). FASTQ sequences were generated, demultiplexed and aligned to the reference genome using the Cell Ranger package (10× Genomics). Gene expression was further analyzed using Seurat V3 (Stuart et al., 2019). Cells that have unique feature counts below 200 or over 2,500, or that contain more than 5% mitochondrial counts, were removed. Gene expression values were log-normalized using the function NormalizedData. Genes mapped to immunoglobulin loci were removed and data were scaled before identifying highly variable genes using FindVariableGenes. Linear dimensional reduction was performed based on scaled data and clusters were identified using the functions FindNeighbors and Findclusters based on top 10 principal components with resolution set to 0.5. Clusters exhibiting T cell features were removed before final linear dimensional reduction and clusters calling. Clusters visualizations were performed with UMAP using the top 10 principal components. Gene ontology analysis was performed using the package Enrichr (Xie et al., 2021), based on GO_Biological_Process_2021.

B cell receptor repertoire analysis

Reconstructed V(D)J sequences from Cell Ranger output were further analyzed by using the Change-O package (Gupta et al., Bioinformatics 31, 3356-3358, 2015). V(D)J germline assignments were performed with IgBLAST (Ye et al., 2013) using the IMGT reference gene database (Lefranc et al., Nucleic Acids Research 43, D413-D422, 2015). Cells with multiple V(D)J sequences were assigned to the most abundant V(D)J sequence by UMI count. Cells with non-functional V(D)J were filtered prior to clonal grouping. Sequences were first grouped by IGHV and IGHJ gene annotations and junction length, and nucleotide hamming distance was calculated. Grouping of B cell clones was additionally performed by setting the hamming distance threshold to 0.16. Clonal groups were corrected based on light chain data. Multiple CDR3 properties and SHM rates were calculated using the packages Alakazam and SHazaM (Gupta et al., Bioinformatics 31, 3356-3358, 2015). IGHV and IGHJ gene selection were visualized by treemap, in which each output square was first grouped by IGHV genes and then further subdivided according to IGHJ selection. The size of each square represents the UMI counts. V(D)J assignment and clonal grouping were integrated with gene expression data according to cell barcodes. Clonal types characterization based on frequency ranges and clonal overlap calculation between clusters were performed using the ScRepertoire package (Borcherding et al., Nature 294, 88-90, 2020).

Recombinant monoclonal antibody production

From the ERV-reactive B-1 BCR repertoire, V(D)J sequences encoded by IGHV1-53, IGHV3-6, IGHV6-3 and IGHV7-3 were extracted. A consensus sequence of the largest clones from each IGHV genes was generated for recombinant DNA synthesis (IDT). Monoclonal antibodies were produced according to a previously published protocol (Vazquez-Lombardi et al., In Antibody Engineering: Methods and Protocols, D. Nevoltris, and P. Chames, eds. (New York, NY: Springer New York), pp. 313-334, 2018). Heavy chain V(D)J sequences were cloned into pRVL-2 vectors (γ2c) (Addgene) and light chain sequences were cloned into pRVL-1 vectors (K) (Addgene). Heavy and light chain pairings were as follows: IGHV1-53 heavy chain with IGKV4 light chain, IGHV3-6 heavy chain with IGLV1 light chain, IGHV6-3 heavy chain with IGKV14 light chain, and IGH7-3 heavy chain with IGLV1 light chain. Heavy and light chain expression plasmids (100 μg total) were transfected at a ratio of 2:1 into 100 mL of Expi293F™ cell culture (Gibco) using the ExpiFectamine™ 293 Transfection Kit (ThermoFisher) according to the manufacturer's manual. On day 7 after transfection, supernatants were harvested and adjusted to the salt concentration of 100 nM NaCl and 20 mM Tris-HCl (pH8), followed by 2-hour RT-incubation with 2 mL of Protein G agarose (ThermoFisher) with gentle rotation. The mixtures were then loaded onto gravity-flow Econo-Pac© Chromatography Columns (Bio-Rad). Packed beads were washed twice with 5× CV of PBS. Bound antibodies were eluted with 4 mL of elution buffer (100 mM glycine, 100 mM NaCl, pH 2.7) into 15 mL collection tubes each containing 1 mL of neutralization buffer (1 M Tris-HCl, pH 7.6). Elutes were concentrated by centrifugation using 30,000 MWCO filter units (GE Healthcare). Buffer exchange was performed in the same filter unit by adding three times the elution volume of PBS. The concentration of mAb was determined by absorbance at 280 nm. For production of domain-swapped mAb, CDR3 sequences were exchanged by seamless DNA cloning using Gibson Assembly® Master Mix (NEB). mAb production was scaled down by using 2.5 mL Expi293F™ cell culture. Purification of antibodies was performed in Poly-Prep® Chromatography Columns (Bio-Rad).

### Western Blotting

Purified recombinant proteins were denatured and deglycosylated by 3-N-Acetylglucosaminidase S (NEB), PNGase F (NEB) or O-Glycosidase & Neuraminidase Bundle (NEB) according to the manufacturer's protocols. Samples were then subjected to electrophoresis in 12% polyacrylamide gel followed by transfer onto PVDF membranes. PVDF blots were probed with HRP conjugated anti-His tag antibody (Cell signaling) ( 1/1000 dilution), or with 3 μg/mL monoclonal antibody followed by detection using a 1/1000 dilution of HRP-conjugated anti-mouse IgG secondary antibody (Southern Biotech). Blocking of the membrane and antibody dilutions were all performed using Carbo-free blocking solution (Vector Lab). Blots were developed using Pierce™ ECL Western Blotting Substrate or SuperSignal™ West Pico PLUS Chemiluminescent Substrate (ThermoFisher).

Mice in vivo stimulation

Mice aged 6-8 weeks were i.p. injected with R848 (InvivoGen), LPS (InvivoGen) or CpG (TriLink BioTechnologies) at a dose of 18 μg in 100 μL of PBS per mouse. Sera were collected by retro-orbital bleed prior to stimulation and then daily for six days. Mice aged 10 weeks were i.p. injected with 10⁶ ERV particles or 5×10⁵ isolated Tlr7-T cells (as described in cell isolation method) in 100 μL of PBS per mouse or i.n. infected with 30 pfu of PR8 (kindly gifted by Hideki Hasegawa, National Institute of Infectious Diseases, Tokyo) in 100 μL PBS per mouse. Sera were collected by retro-orbital bleed on day 0 and on day 6 following stimulation.

ERV in vitro infection

DFJ8 cells were seeded at 1×10⁵ cells per well with 500 μL DMEM in each well of a 24-well-plate. On the day of infection, 5×10⁵ of ERV particles (M.O.I. =5) were incubated with 2.5 μg of mAb, with or without 2.5 μL of Rag^{-/-} MD4 Tg serum in OptiMEM in a total volume of 50 μL. After a 1-hour incubation at 37° C., DMEM in the 24-well-plate were removed and replaced by 50 μL of OptiMEM, each 50 μL mixture containing ERV and mAb was then added to a DFJ8 cells-containing well to reach a final volume of 100 μL. After a 1-hour incubation at 37° C., the supernatant was removed and 500 μL of fresh pre-warmed DMEM were added. At 48 hours post-infection, cells were harvested for staining with mAb 573 to quantify the percentage of infected cells.

### Quantification and Statistical Analysis

For each plot, values were plotted as individual data points, if applicable, with mean and standard deviation (S.D.) calculated for the respective measured parameter. Statistical analyses were conducted by GraphPad Prism software using one-way or two-way ANOVA with Sidik's multiple comparisons tests, either within each group or using the mean value of the negative control. Paired and unpaired t-tests with Holm-Sidik's multiple comparisons were performed when appropriate. All statistical analyses are indicated in the respective figure legend. Quantification methods for RT-qPCR, ELISA, and flow cytometry are described in the above methods. For mouse experiments, mice used in each experiment were strictly age- and gender-matched. Both male and female mice were used in this study. Sex variation was determined to not affect anti-ERV antibody levels at steady state in pilot experiments. Age was determined to affect anti-ERV antibody levels at steady state. In FIGS. 5C and 10D, mice between the ages of 6 and 16 weeks were used. In FIG. 10B, 10-week-old mice were used and in all other in vivo experiments, mice between the ages of 6 to 8 weeks were used. In the experiments monitoring for infectious ERV emergence (FIGS. 5B, 12 and 18C), sample sizes were determined by the number of offspring from each breeding. Except for cleaning of sequencing results as described in these methods, no other exclusion criteria were applied to any results.

### Enumerated Embodiments

In some aspects, the present invention is directed to the following non-limiting embodiments:
Embodiment 1: An isolated antigen-binding protein comprising a light chain polypeptide and a heavy chain polypeptide. In certain embodiments, the light chain polypeptide comprises a complementarity determining region (CDR) 1 comprising the amino acid sequence of SEQ ID NO: 1; a CDR2 comprising the amino acid sequence of SEQ ID NO: 2; and a CDR3 comprising the amino acid sequence of SEQ ID NO: 3. In certain embodiments, the heavy chain polypeptide comprises a CDR 1 comprising the amino acid sequence of SEQ ID NO: 4; a CDR2 comprising the amino acid sequence of SEQ ID NO: 5; and a CDR3 comprising the amino acid sequence of SEQ ID NO: 6.
Embodiment 2: The antigen-binding protein of Embodiment 1, wherein the light chain polypeptide comprises the amino acid sequence of SEQ ID NO: 7, and wherein the heavy chain polypeptide comprises the amino acid sequence of SEQ ID NO: 8.
Embodiment 3: The antigen-binding protein of Embodiment 1, wherein the antigen-binding protein comprises at least one selected from the group consisting of a polyclonal antibody, a monoclonal antibody, a variable fragment (Fv), an antigen-binding fragment (Fab or F(ab)₂), a single chain antibody (scFv), a camelid antibody and a humanized antibody.
Embodiment 4: The antigen-binding protein of Embodiment 1, wherein the antigen-binding protein binds to viral proteins of two or more viruses from different viral families.
Embodiment 5: The antigen-binding protein of Embodiment 4, wherein the antigen-binding protein binds to a viral protein from a Orthomyxoviridae family virus and a viral protein from a Coronaviridae family virus.
Embodiment 6: The antigen-binding protein of Embodiment 4, wherein the antigen-binding protein binds to a viral protein from an influenza virus and a viral protein from severe acute respiratory syndrome coronavirus 2 (SARS-CoV-2).
Embodiment 7: The antigen-binding protein of Embodiment 1, wherein the antigen-binding protein binds to a glycosylation feature specific to a viral protein which is not found in a protein of a host of the virus.
Embodiment 8: The antigen-binding protein of Embodiment 7, wherein the glycosylation feature comprises an N-acetylglucosamine (GlcNAc) residue on a non-reducing end of an oligosaccharide.
Embodiment 9: A composition comprising the antigen-binding protein of Embodiment 1; and at least one pharmaceutically acceptable carrier.
Embodiment 10: The composition of Embodiment 9, further comprising:
   a compound for treating, preventing and/or ameliorating an infection by an influenza virus in a subject; and/or
   a compound for treating, preventing and/or ameliorating an infection by SARS-CoV-2 virus in a subject.
Embodiment 11: A nucleic acid encoding the antigen-binding protein of Embodiment 1.
Embodiment 12: The nucleic acid of Embodiment 11, wherein the nucleic acid comprises a first segment comprising the nucleotide sequence of SEQ ID NO: 9, and a second segment comprising the nucleotide sequence of SEQ ID NO: 10.
Embodiment 13: A method of preventing, treating and/or ameliorating an infection by a virus in a subject in need thereof, the method comprising administering to the subject an effective amount of the antibody of Embodiment 1.
Embodiment 14: The method of Embodiment 13, wherein a protein of the virus has a glycosylation feature is not found in a protein of a host of the virus, and wherein the antibody specifically recognizes the glycosylation feature.
Embodiment 15: The method of Embodiment 14, wherein the glycosylation feature comprises an N-acetylglucosamine (GlcNAc) residue on a non-reducing end of an oligosaccharide.
Embodiment 16: The method of Embodiment 13, wherein the virus is an Orthomyxoviridae family virus or a Coronaviridae family virus.
Embodiment 17: The method of Embodiment 13, wherein the virus is an influenza virus or SARS-CoV-2 virus.
Embodiment 18: The method of Embodiment 17, wherein the subject is further administered a second compound suitable for treating, preventing and/or ameliorating an influenza virus infection, or a second compound suitable for treating, preventing and/or ameliorating COVID-19.
Embodiment 19: The method of Embodiment 13, wherein the subject is a mammal.
Embodiment 20: The method of Embodiment 13, wherein the subject is a human.

The disclosures of each and every patent, patent application, and publication cited herein are hereby incorporated herein by reference in their entireties.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. An isolated antigen-binding protein comprising a light chain polypeptide and a heavy chain polypeptide, wherein
the light chain polypeptide comprises:
a complementarity determining region (CDR) 1 comprising the amino acid sequence of SEQ ID NO: 1;
a CDR2 comprising the amino acid sequence of SEQ ID NO: 2; and
a CDR3 comprising the amino acid sequence of SEQ ID NO: 3, and
the heavy chain polypeptide comprises:
a CDR 1 comprising the amino acid sequence of SEQ ID NO: 4;
a CDR2 comprising the amino acid sequence of SEQ ID NO: 5; and
a CDR3 comprising the amino acid sequence of SEQ ID NO: 6.

2. The antigen-binding protein of claim 1, wherein the light chain polypeptide comprises the amino acid sequence of SEQ ID NO: 7, and wherein the heavy chain polypeptide comprises the amino acid sequence of SEQ ID NO: 8.

3. The antigen-binding protein of claim 1, wherein the antigen-binding protein comprises at least one selected from the group consisting of a polyclonal antibody, a monoclonal antibody, a variable fragment (Fv), an antigen-binding fragment (Fab or F(ab)₂), a single chain antibody (scFv), a camelid antibody and a humanized antibody.

4. The antigen-binding protein of claim 1, wherein the antigen-binding protein binds to viral proteins of two or more viruses from different viral families.

5. The antigen-binding protein of claim 4, wherein the antigen-binding protein binds to a viral protein from a Orthomyxoviridae family virus and a viral protein from a Coronaviridae family virus.

6. The antigen-binding protein of claim 4, wherein the antigen-binding protein binds to a viral protein from an influenza virus and a viral protein from severe acute respiratory syndrome coronavirus 2 (SARS-CoV-2).

7. The antigen-binding protein of claim 1, wherein the antigen-binding protein binds to a glycosylation feature specific to a viral protein which is not found in a protein of a host of the virus.

8. The antigen-binding protein of claim 7, wherein the glycosylation feature comprises an N-acetylglucosamine (GlcNAc) residue on a non-reducing end of an oligosaccharide.

9. A composition comprising the antigen-binding protein of claim 1 and at least one pharmaceutically acceptable carrier.

10. The composition of claim 9, further comprising:
a compound for treating, preventing, or ameliorating an infection by an influenza virus in a subject; or
a compound for treating, preventing, or ameliorating an infection by SARS-CoV-2 virus in a subject.

11. A nucleic acid encoding the antigen-binding protein of claim 1.

12. The nucleic acid of claim 11, wherein the nucleic acid comprises a first segment comprising the nucleotide sequence of SEQ ID NO: 9, and a second segment comprising the nucleotide sequence of SEQ ID NO: 10.

13. A method of preventing, treating, or ameliorating an infection by a virus in a subject in need thereof, the method comprising administering to the subject an effective amount of the antibody of claim 1.

14. The method of claim 13, wherein a protein of the virus has a glycosylation feature is not found in a protein of a host of the virus, and wherein the antibody specifically recognizes the glycosylation feature.

15. The method of claim 14, wherein the glycosylation feature comprises an N-acetylglucosamine (GlcNAc) residue on a non-reducing end of an oligosaccharide.

16. The method of claim 13, wherein the virus is an Orthomyxoviridae family virus or a Coronaviridae family virus.

17. The method of claim 13, wherein the virus is an influenza virus or SARS-CoV-2 virus.

18. The method of claim 17, wherein the subject is further administered a second compound suitable for treating, preventing and/or ameliorating an influenza virus infection, or a second compound suitable for treating, preventing, and/or ameliorating COVID-19.

19. The method of claim 13, wherein the subject is a mammal.

20. The method of claim 13, wherein the subject is a human.


SEQUENCE LISTING (27 sequences)

SEQ ID NO: 1 - protein, 6 aa, Mus musculus
QDINSY

SEQ ID NO: 2 - skipped

SEQ ID NO: 3 - protein, 9 aa, Mus musculus
LQYDEFPYT

SEQ ID NO: 4 - protein, 8 aa, Mus musculus
GFTFSNYW

SEQ ID NO: 5 - protein, 10 aa, Mus musculus
IRLKSDNYAT

SEQ ID NO: 6 - protein, 9 aa, Mus musculus
TDITGPIDY

SEQ ID NO: 7 - protein, 129 aa, Mus musculus
MDMRTPAQFLGILLLWFPGIKCDIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIK

SEQ ID NO: 8 - protein, 137 aa, Mus musculus
MDLRLSCAFIIVLLKGVQSEVKLEESGGGLVQPGGSMKLSCVASGFTFSNYWMNWVRQSPEKGLEWVAQIRLKSDNYATHYAESVKGRFTISRDDSKSSVYLQMNNLRAEDTGIYYCTDITGPIDYWGQGTTLTVSS

SEQ ID NO: 9 - DNA, 388 bp, Mus musculus
  source 1..388: mol_type = genomic DNA
atggacatgaggacccctgctcagtttcttggaatcttgttgctctggtttccaggtatcaaatgtgacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaagctggaaataaaac

SEQ ID NO: 10 - DNA, 412 bp, Mus musculus
  source 1..412: mol_type = genomic DNA
atggacttgagactgagctgtgcttttattattgttcttttaaaaggggtccagagtgaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctcaaattagattgaaatctgataattatgcaacacattatgcggagtctgtgaaagggaggttcaccatctcaagagatgattccaaaagtagtgtctacctgcaaatgaacaacttaagggctgaagacactggaatttattactgcacagatataactgggccgattgactactggggccaaggcaccactctcacagtctcctcag

SEQ ID NO: 11 - RNA, 22 nt, synthetic construct
  source 1..22: mol_type = other RNA
ccagggaccaccgacccaccgt

SEQ ID NO: 12 - RNA, 22 nt, synthetic construct
  source 1..22: mol_type = other RNA
tagtcggtcccggtaggcctcg

SEQ ID NO: 13 - RNA, 19 nt, synthetic construct
  source 1..19: mol_type = other RNA
gaaggtcggtgtgaacgga

SEQ ID NO: 14 - RNA, 20 nt, synthetic construct
  source 1..20: mol_type = other RNA
gttagtggggtctcgctcct

SEQ ID NO: 15 - RNA, 29 nt, synthetic construct
  source 1..29: mol_type = other RNA
tggctgactgaggctagaaaagagactgt

SEQ ID NO: 16 - RNA, 22 nt, synthetic construct
  source 1..22: mol_type = other RNA
gctttttggacaggtaggccac

SEQ ID NO: 17 - RNA, 23 nt, synthetic construct
  source 1..23: mol_type = other RNA
ggcctccctcctgaccttagacg

SEQ ID NO: 18 - RNA, 23 nt, synthetic construct
  source 1..23: mol_type = other RNA
taaggggattaggaggtcccgcg

SEQ ID NO: 19 - RNA, 26 nt, synthetic construct
  source 1..26: mol_type = other RNA
ggctttagtcctgactcaacaatacc

SEQ ID NO: 20 - RNA, 50 nt, synthetic construct
  source 1..50: mol_type = other RNA
cgcgggacctcctaatccccttaccagctaaaaccactagaatacgagcc

SEQ ID NO: 21 - RNA, 20 nt, synthetic construct
  source 1..20: mol_type = other RNA
tttccataggctccgccccc

SEQ ID NO: 22 - RNA, 23 nt, synthetic construct
  source 1..23: mol_type = other RNA
ggggcggagcctatggaaaaacg

SEQ ID NO: 23 - RNA, 23 nt, synthetic construct
  source 1..23: mol_type = other RNA
cgtctaaggtcaggagggaggcc

SEQ ID NO: 24 - protein, 120 aa, Mus musculus
EVKLEESGGGLVQPGGSMKLSCVASGFTFSNYWMNWVRQSPEKGLEWVAQIRLKSDNYATHYAESVKGRFTISRDDSKSSVYLQMNNLRAEDTGIYYCTDITGPIDYWGQGTTLTVSSEV

SEQ ID NO: 25 - protein, 120 aa, Mus musculus
EVKLVESGGGLVQPGGSLSLSCAASGFTFTDYYMSWVRQPPGKALEWLGFIRNKANGYTTEYSASVKGRFTISRDNSQSILYLQMNALRAEDSATYYCARYYYGSSYAMDYWGQGTSVTV

SEQ ID NO: 26 - protein, 120 aa, Mus musculus
EVKLEESGGGLVQPGGSMKLSCVASGFTFSNYWMDWVRQSPEKGLEWVAEIRLKSDNFATHYAESVKGRFTISRDDSKSSVYLQMNNLRAEDTGIYYCVDLSWFAYWGQGTEVKLEESGG

SEQ ID NO: 27 - protein, 120 aa, Mus musculus
QVQLQQPGTELVKPGASVKLSCKASGYTFTSYWIHWVKQRPGQGLEWIGNINPPNGGTNYNEKFKNKATLTVDQSSSTAYMQLSSLTSEDSAVYYCARKEYLWYFDVWGTGTTVTVSSQV